(19) United States
(12) Patent Application Publication
(10) Pub. No.: US 2026/0219161 A1
(43) Pub. Date: Jul. 30, 2026

(54) BACK-ILLUMINATED IMAGING ELEMENT, FLOW CHANNEL UNIT FOR ANALYZING BIOLOGICAL SAMPLE, AND BIOLOGICAL SAMPLE ANALYSIS SYSTEM

(71) Applicant: SONY SEMICONDUCTOR SOLUTIONS CORPORATION, Kanagawa (JP)

(72) Inventors: Kengo NAGATA, Kanagawa (JP); Shinta KOBAYASHI, Kanagawa (JP); Atsuhiro ANDO, Kanagawa (JP); Yoshiaki MASUDA, Kanagawa (JP); Takuo ENDO, Kanagawa (JP); Toshiki SAKAMOTO, Kumamoto (JP); Yukari TAGUCHI, Kumamoto (JP); Ryosuke OOGAMI, Kumamoto (JP); Naoki NISHI, Kanagawa (JP); Koji NUNOMURA, Kanagawa (JP); Motoaki NAKAMURA, Kanagawa (JP); Shinichiro NOUDO, Kumamoto (JP); Takafumi MORIKAWA, Kanagawa (JP); Seiichi FUNAKOSHI, Kanagawa (JP); Junya OMORI, Kanagawa (JP); Yuji TANAKA, Kumamoto (JP); Tomoki HIRAMATSU, Kanagawa (JP); Norihiro KUBO, Kanagawa (JP); Ayaka HIYAMA, Tokyo (JP); Atsushi TODA, Kanagawa (JP)

(73) Assignee: SONY SEMICONDUCTOR SOLUTIONS CORPORATION, Kanagawa (JP)

(21) Appl. No.: 19/145,020

(22) PCT Filed: Dec. 25, 2023
(86) PCT No.: PCT/JP2023/046365

§ 371 Date: Jul. 1, 2025

(30) Foreign Application Priority Data
Jan. 13, 2023 (JP) 2023-003751

(51) Int. Cl. G01N 15/1434; G01N 15/01; G01N 15/10

(52) U.S. Cl. CPC G01N 15/1434; G01N 15/01; G01N 2015/1006

(57) ABSTRACT

An object of the present disclosure is to provide a technique for improving fluorescence detection accuracy in biological sample analysis. The present disclosure provides a back-illuminated imaging element including a plurality of pixel units, each of which includes at least an analyte holding unit configured to hold an analyte and a fluorescence detection unit that detects fluorescence generated by irradiating the analyte with excitation light. In addition, the present disclosure also provides a flow channel unit for analyzing a biological sample including the back-illuminated imaging element. In addition, the present disclosure also provides a biological sample analysis system that analyzes a biological sample using the flow channel unit for analyzing a biological sample.

### TECHNICAL FIELD

The present disclosure relates to a back-illuminated imaging element, a flow channel unit for analyzing a biological sample, and a biological sample analysis system.

### BACKGROUND ART

Various proposals have been made for devices for detecting or analyzing biological materials. In such devices, light from biological materials is often detected. The light is often weak, and it is desirable to increase detection accuracy. With respect to such devices, for example, Patent Document 1 below discloses “A chip for detecting a biologically derived material, the chip including a plurality of pixels, in which each of the pixels includes at least a holding surface that holds the biologically derived material and a photoelectric conversion unit provided below the holding surface and provided on a semiconductor substrate, and a color mixture suppression unit is provided between the pixels.” (claim 1).

In addition, DNA is often a target of detection. Examples of a device for analyzing a base sequence of DNA include a DNA sequencer. The DNA sequencer identifies, for example, types of bases constituting DNA by fluorescence. The DNA sequencer is configured to be capable of radiating excitation light, and can further include a detection unit including, for example, transparent glass that transmits the excitation light, a flow channel through which a sample flows, a nanowell, an optical filter, and a photodiode. The nanowell has, for example, a well shape sized to accommodate fragmented DNA.

### CITATION LIST

### Patent Document

Patent Document 1: Japanese Patent Application Laid-Open No. 2020-85666

### SUMMARY OF THE INVENTION

### Problems to be Solved by the Invention

In order to increase processing speed of the DNA sequencer, for example, it is conceivable to increase the number of pixels of an imaging element that detects fluorescence, but it is not desirable to increase size of the imaging element, for example, from the viewpoint of cost and the like. It is therefore conceivable to reduce size of a unit pixel (particularly the photodiode).

Distances between the pixels, however, become short due to the reduction of the size of the unit pixel, which causes optical crosstalk to generate noise, and a fluorescence signal derived from DNA is buried in the noise. This can lead to a decrease in fluorescence detection accuracy.

In addition, a large filter film thickness for cutting off excitation light can also cause a decrease in the fluorescence detection accuracy due to the optical crosstalk. For example, in a case where the filter film thickness is large, a distance between the detection unit and a fluorophore is increased, and as a result, fluorescence diffusing in oblique directions enters adjacent pixels and becomes noise. Such optical crosstalk deteriorates an S/N ratio.

In addition, the excitation light for generating fluorescence can also cause a decrease in the fluorescence detection accuracy. For example, in a case where a main light beam of the excitation light is directly incident on the sensor, an excitation light component becomes noise, which can also cause a decrease in the fluorescence detection accuracy.

An object of the present disclosure, therefore, is to provide a technique for improving fluorescence detection accuracy in biological sample analysis.

### Solutions to Problems

The present disclosure provides
a back-illuminated imaging element including:
a plurality of pixel units including at least:
an analyte holding unit configured to hold an analyte; and
a fluorescence detection unit that detects fluorescence generated by irradiating the analyte with excitation light.

The analyte holding unit may have a shape of a well, and
the detection unit may be provided in such a way as to cover a side surface of the well in addition to a bottom of the well.

In the back-illuminated imaging element, a trench may be provided between the pixel units.

In the imaging element, two or more wells may be connected to each other in such a way as to form a column structure.

Each pixel unit may be provided with an electrode pair to which a voltage is applied in such a way as to adjust a position of the analyte.

Each pixel unit may be provided with an excitation light blocking unit that prevents the excitation light from reaching the detection unit.

The excitation light blocking unit may include a multilayer film reflection filter.

The multilayer film reflection filter may be disposed between the analyte holding unit and the detection unit. Each pixel unit may further include an excitation light detection unit that detects the excitation light.

The back-illuminated imaging element may be configured to process a signal obtained by the fluorescence detection unit using a signal obtained by the excitation light detection unit.

The excitation light blocking unit may include a polarizer, a plasmon filter, a metamaterial, or a multilayer film having a Fabry-Perot structure.

The excitation light blocking unit may be configured to transmit the fluorescence.

The excitation light blocking unit may include a polarizer, and
the excitation light may be polarized light.

The excitation light blocking unit may include a polarizer, and
one polarizer may be provided in such a way as to cover detection units of two or more pixel units.

The fluorescence detection unit may include two or more photodiodes.

The two or more photodiodes may be arranged in such a way as to form a vertically stacked structure between the analyte holding unit and a wiring layer.

A photodiode closer to the wiring layer among the two or more photodiodes may be configured to detect fluorescence of a longer wavelength.

The two or more photodiodes may form a two-layer structure or a three-layer structure.

In addition, the present disclosure also provides
a flow channel unit for analyzing a biological sample, the flow channel unit including:
a back-illuminated imaging element including a plurality of pixel units, each of which includes at least an analyte holding unit configured to hold an analyte and a fluorescence detection unit that detects fluorescence generated by irradiating the analyte with excitation light; and
a flow channel that supplies the biological sample to the analyte holding unit.

In addition, the present disclosure also provides
a biological sample analysis system that analyzes a biological sample using a flow channel unit for analyzing a biological sample, the flow channel unit including:
a back-illuminated imaging element including a plurality of pixel units, each of which includes at least an analyte holding unit configured to hold an analyte and a fluorescence detection unit that detects fluorescence generated by irradiating the analyte with excitation light; and
a flow channel that supplies the biological sample to the analyte holding unit.

### BRIEF DESCRIPTION OF DRAWINGS

FIG. 1 is a schematic diagram illustrating a configuration example of a back-illuminated imaging element according to the present disclosure.

FIG. 2A is a diagram for explaining sizes of a well and a pixel unit.

FIG. 2B is another diagram for explaining the size of the well and the pixel unit.

FIG. 2C is a schematic diagram illustrating a configuration example of an electronic reading unit.

FIG. 3 is a schematic diagram illustrating another configuration example of the back-illuminated imaging element according to the present disclosure.

FIG. 4A is a schematic diagram illustrating another configuration example of the back-illuminated imaging element according to the present disclosure.

FIG. 4B is a schematic diagram illustrating another configuration example of the back-illuminated imaging element according to the present disclosure.

FIG. 4C is a schematic diagram illustrating another configuration example of the back-illuminated imaging element according to the present disclosure.

FIG. 5 is a schematic diagram illustrating another configuration example of the back-illuminated imaging element according to the present disclosure.

FIG. 6 is a schematic diagram illustrating a configuration example of a back-illuminated imaging element according to the present disclosure including a multilayer film reflection filter.

FIG. 7A is a schematic diagram for explaining a configuration example of the multilayer film reflection filter.

FIG. 7B is another schematic diagram for explaining the configuration example of the multilayer film reflection filter.

FIG. 8 is a schematic diagram illustrating another configuration example of the back-illuminated imaging element according to the present disclosure including a multilayer film reflection filter.

FIG. 9 is a schematic diagram illustrating another configuration example of the back-illuminated imaging element according to the present disclosure including a multilayer film reflection filter.

FIG. 10 is a schematic diagram illustrating another configuration example of the back-illuminated imaging element according to the present disclosure.

FIG. 11 is a schematic cross-sectional view of another configuration example of the back-illuminated imaging element according to the present disclosure.

FIG. 12 is a schematic cross-sectional view of another configuration example of the back-illuminated imaging element according to the present disclosure.

FIG. 13 is a schematic cross-sectional view of another configuration example of the back-illuminated imaging element according to the present disclosure.

FIG. 14 is a schematic cross-sectional view of another configuration example of the back-illuminated imaging element according to the present disclosure.

FIG. 15 is a schematic cross-sectional view of another configuration example of the back-illuminated imaging element according to the present disclosure.

FIG. 16 is a schematic cross-sectional view of another configuration example of the back-illuminated imaging element according to the present disclosure.

FIG. 17 is a schematic cross-sectional view of another configuration example of the back-illuminated imaging element according to the present disclosure.

FIG. 18 is a schematic cross-sectional view of another configuration example of the back-illuminated imaging element according to the present disclosure.

FIG. 19 is a schematic cross-sectional view of another configuration example of the back-illuminated imaging element according to the present disclosure.

FIG. 20 is a schematic cross-sectional view of another configuration example of the back-illuminated imaging element according to the present disclosure.

FIG. 21 is a schematic cross-sectional view of another configuration example of the back-illuminated imaging element according to the present disclosure.

FIG. 22 is a schematic cross-sectional view of another configuration example of the back-illuminated imaging element according to the present disclosure.

FIG. 23 is a schematic cross-sectional view of another configuration example of the back-illuminated imaging element according to the present disclosure.

FIG. 24 is a schematic cross-sectional view of another configuration example of the back-illuminated imaging element according to the present disclosure.

FIG. 25 is a schematic cross-sectional view of another configuration example of the back-illuminated imaging element according to the present disclosure.

FIG. 26 is a schematic cross-sectional view of another configuration example of the back-illuminated imaging element according to the present disclosure.

FIG. 27 is a schematic cross-sectional view of another configuration example of the back-illuminated imaging element according to the present disclosure.

FIG. 28 is a schematic cross-sectional view of another configuration example of the back-illuminated imaging element according to the present disclosure.

FIG. 29 is a schematic cross-sectional view of another configuration example of the back-illuminated imaging element according to the present disclosure.

FIG. 30 is a schematic cross-sectional view of another configuration example of the back-illuminated imaging element according to the present disclosure.

FIG. 31 is a schematic cross-sectional view of another configuration example of the back-illuminated imaging element according to the present disclosure.

FIG. 32 is a schematic cross-sectional view of another configuration example of the back-illuminated imaging element according to the present disclosure.

FIG. 33A is a schematic cross-sectional view of another configuration example of the back-illuminated imaging element according to the present disclosure.

FIG. 33B is a schematic cross-sectional view of another configuration example of the back-illuminated imaging element according to the present disclosure.

FIG. 34A is a schematic cross-sectional view of another configuration example of the back-illuminated imaging element according to the present disclosure.

FIG. 34B is a schematic cross-sectional view of another configuration example of the back-illuminated imaging element according to the present disclosure.

FIG. 35 is a diagram illustrating a configuration example of a trench.

FIG. 36A is a schematic diagram illustrating another configuration example of the back-illuminated imaging element according to the present disclosure.

FIG. 36B is a schematic diagram illustrating another configuration example of the back-illuminated imaging element according to the present disclosure.

FIG. 36C is a schematic diagram illustrating another configuration example of the back-illuminated imaging element according to the present disclosure.

FIG. 37 is a diagram illustrating examples of a pattern of a polarizer.

FIG. 38 is a schematic diagram illustrating another configuration example of the back-illuminated imaging element according to the present disclosure.

FIG. 39 is a schematic diagram illustrating another configuration example of the back-illuminated imaging element according to the present disclosure.

FIG. 40 is a schematic diagram illustrating another configuration example of the back-illuminated imaging element according to the present disclosure.

FIG. 41 is a schematic diagram illustrating another configuration example of the back-illuminated imaging element according to the present disclosure.

FIG. 42 is a schematic diagram illustrating another configuration example of the back-illuminated imaging element according to the present disclosure.

FIG. 43 is a diagram illustrating examples of a film having an FP structure.

FIG. 44 is a schematic diagram illustrating another configuration example of the back-illuminated imaging element according to the present disclosure.

FIG. 45 is a schematic diagram illustrating another configuration example of the back-illuminated imaging element according to the present disclosure.

FIG. 46 is a schematic diagram illustrating another configuration example of the back-illuminated imaging element according to the present disclosure.

FIG. 47 is a schematic diagram illustrating another configuration example of the back-illuminated imaging element according to the present disclosure.

FIG. 48 is a diagram illustrating a result of verification of sensitivity

FIG. 49 is a diagram illustrating another result of the verification of the sensitivity

FIG. 50A is a schematic view for explaining a method for manufacturing the back-illuminated imaging element according to the present disclosure.

FIG. 50B is another schematic view for explaining the method for manufacturing the back-illuminated imaging element according to the present disclosure.

FIG. 50C is another schematic view for explaining the method for manufacturing the back-illuminated imaging element according to the present disclosure.

FIG. 51A is a schematic view for explaining another method for manufacturing the back-illuminated imaging element according to the present disclosure.

FIG. 51B is another schematic view for explaining the other method for manufacturing the back-illuminated imaging element according to the present disclosure.

FIG. 51C is another schematic view for explaining the other method for manufacturing the back-illuminated imaging element according to the present disclosure.

FIG. 51D is another schematic view for explaining the other method for manufacturing the back-illuminated imaging element according to the present disclosure.

FIG. 52A is a schematic view for explaining another method for manufacturing the back-illuminated imaging element according to the present disclosure.

FIG. 52B is another schematic view for explaining the other method for manufacturing the back-illuminated imaging element according to the present disclosure.

FIG. 52C is another schematic view for explaining the other method for manufacturing the back-illuminated imaging element according to the present disclosure.

FIG. 52D is another schematic view for explaining the other method for manufacturing the back-illuminated imaging element according to the present disclosure.

FIG. 53 is a schematic diagram illustrating a configuration example of a flow channel unit according to the present disclosure.

FIG. 54 is a block diagram illustrating a configuration example of a biological sample analysis system according to the present disclosure.

FIG. 55A is a schematic diagram illustrating a situation in which light passes through the multilayer film reflection filter.

FIG. 55B is a schematic diagram for explaining a method for calculating transmittance T.

FIG. 55C is another schematic diagram for explaining the method for calculating the transmittance T.

FIG. 55D is another schematic diagram for explaining the method for calculating the transmittance T.

### MODE FOR CARRYING OUT THE INVENTION

Preferred modes for carrying out the present disclosure will be described hereinafter. Note that embodiments described below are representative embodiments of the present disclosure, and the scope of the present disclosure is not limited only to these embodiments. Note that the present disclosure will be described in the following order.
1. First Embodiment (Back-Illuminated Imaging Element)
1.1 Configuration Example Relating to Well Structure of PD
1.2 Configuration Example Relating to Excitation Light Blocking Unit
1.3 Configuration Example Relating to Excitation Light Blocking Unit Including Polarizer
1.4 Configuration Example Relating to PD Vertically Stacked Structure
2. Second Embodiment (Flow Channel Unit for Analyzing Biological Sample)
3. Third Embodiment (Biological Sample Analysis System)

### 1. First Embodiment (Back-Illuminated Imaging Element)

The present inventors have found that an imaging element having a specific configuration is useful for improving fluorescence detection accuracy. That is, the present disclosure provides a specific type of imaging element including a plurality of pixel units having the specific configuration. In one implementation, the pixel unit includes at least an analyte holding unit configured to hold an analyte and a fluorescence detection unit that detects fluorescence generated by irradiating the analyte with excitation light, and the imaging element is of a back-illuminated type. The inclusion of the analyte holding unit and the fluorescence detection unit in each pixel and the imaging element being of the back-illuminated type contribute to improvement of the fluorescence detection accuracy. For example, since each pixel includes the analyte holding unit and the fluorescence detection unit, fluorescence derived from minute biomolecules can be accurately detected.

In addition, for example, because a wiring layer is present right above a photodiode in a front-illuminated imaging element, fluorescence is scattered by the wiring layer and is not incident on the photodiode, and a signal can be lost. Since the imaging element in the present disclosure is configured as a back-illuminated type, scattering of fluorescence due to the wiring layer can be prevented.

In the following 1.1 to 1.4, four main configuration examples will be described. Techniques of these configuration examples may be used independently or in combination in the present disclosure. In one implementation, a configuration in 1.3 or 1.4 below may be combined with the configuration example described in 1.1 below. In another implementation, the configuration in 1.3 or 1.4 below may be combined with the configuration example described in 1.2 below.

### 1.1 Configuration Example Relating to Well Structure of PD

### 1.1.1 Example 1-1 (Basic Configuration Example)

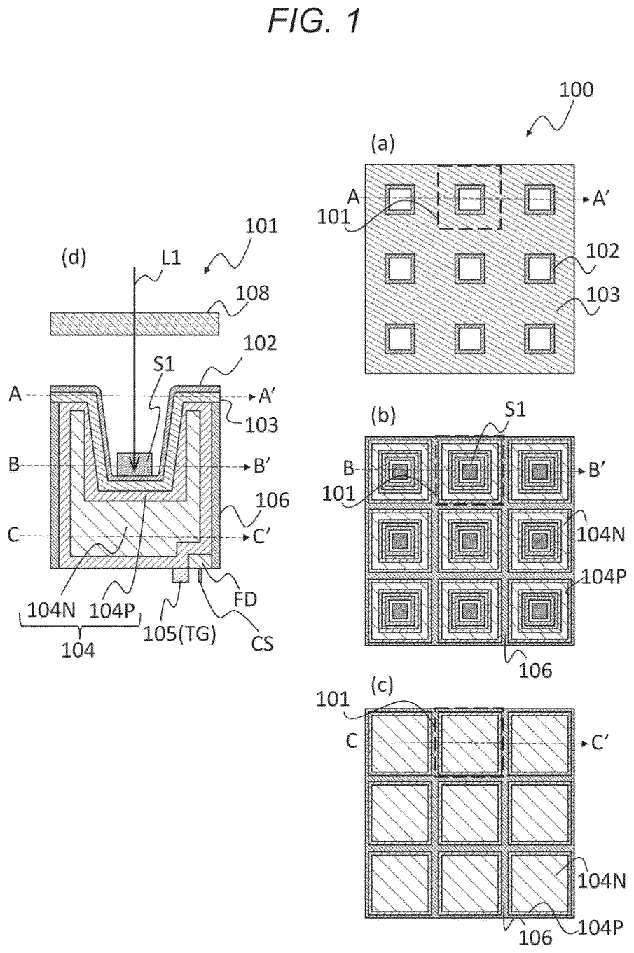

In one implementation, the analyte holding unit may have a shape of a well, and the detection unit may be provided in such a way as to cover a side surface of the well in addition to a bottom of the well. A configuration example of a back-illuminated imaging element in this embodiment will be described hereinafter with reference to FIG. 1. The drawing is a schematic diagram of a structure of the back-illuminated imaging element according to the present disclosure.
(d) on a left side of the drawing is a schematic diagram of a cross section of a pixel unit 101 of the imaging element. The cross section is a cross section in a plane perpendicular to a light receiving surface of the imaging element.
(a) to (c) on a right side of the drawing are schematic diagrams illustrating a part of the light receiving surface of an imaging element 100 in which pixel units 101 are arranged in a lattice pattern, and more specifically, are schematic diagrams of cross sections as follows.
(a) on the right side of the drawing is a schematic diagram of a cross section at a position of broken line A-A′ in (d) of the drawing, and this schematic diagram illustrates a state in which the plurality of pixel units is arranged in a lattice pattern. In addition, the cross section is a cross section in a plane parallel to the light receiving surface of the imaging element.
(b) on the right side of the drawing is a schematic diagram of a cross section at a position of broken line B-B′ in (d) of the drawing, and this schematic diagram illustrates a state in which the plurality of pixel units is arranged in a lattice pattern. In addition, the cross section is a cross section in a plane parallel to the light receiving surface of the imaging element.
(c) on the right side of the drawing is a schematic diagram of a cross section at a position of broken line C-C′ in (d) of the drawing, and this schematic diagram illustrates a state in which the plurality of pixel units is arranged in a lattice pattern. In addition, the cross section is a cross section in a plane parallel to the light receiving surface of the imaging element.

The pixel unit 101 includes a well 103. As illustrated in the drawing, the well 103 may be covered with an insulating film 102.

A shape of an opening of the well 103 (particularly a shape in the plane parallel to the light receiving surface) is rectangular in (a) of the drawing, but may be another polygonal shape, or may be circular, elliptical, or the like, instead.

The well 103 (and the insulating film 102 covering the well) may be configured to hold an analyte, that is, corresponds to the analyte holding unit.

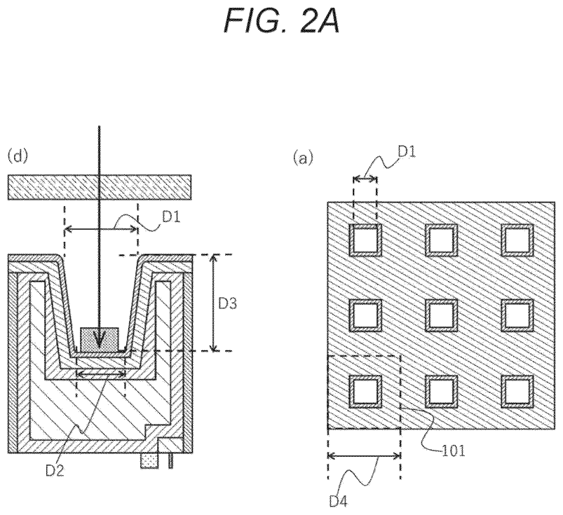

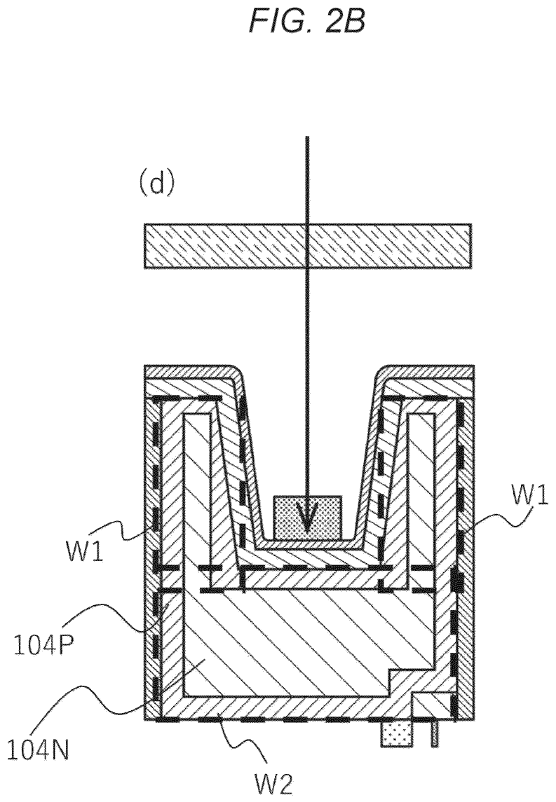

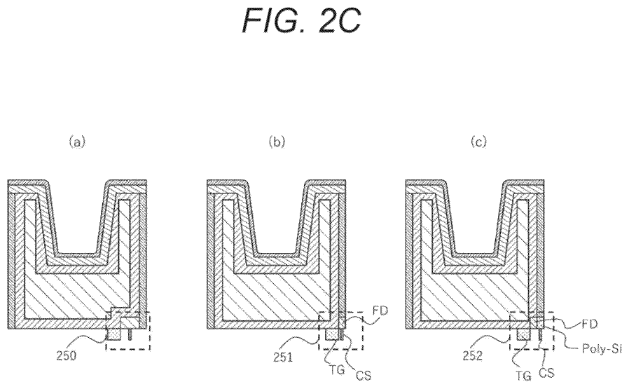

Size of the well 103 and the pixel unit 101 will be described with reference to FIG. 2A.

As illustrated in the drawing, size D1 of the opening of the well 103 may be, for example, 50 nm or more, preferably 100 nm or more, 200 nm or more, or 300 nm or more. The size D1 may be, for example, 200 μm or less, preferably 150 μm or less, 120 μm or less, or 100 μm or less, instead. The size D1 may be appropriately set by those skilled in the art in accordance with, for example, size of the analyte. In a case where the analyte is a cell (size on the order of tens of μm), for example, the size D1 may be, for example, from 1 μm to 200 μm, in particular from 10 μm to 100 μm. In a case where the analyte is a cellular constituent such as a nucleic acid or a protein, the size D1 may be, for example, from 50 nm to 1000 nm, in particular from 100 nm to 900 nm. The size D1 may mean, for example, a length of one side in a case where the shape of the opening of the well is a square, length of a long sides in a case where the shape of the opening is a rectangle, a length of the longest side in a case where the shape of the opening is another rectangle, a longest diagonal distance in a case where the shape of the opening is a polygon with five or more sides, a diameter in a case where the shape of the opening is a circle, and a length of a major axis in a case where the shape of the opening is an ellipse.

Size D2 of a bottom of the well 103 may be, for example, 50 nm or more, preferably 100 nm or more, 200 nm or more, or 300 nm or more. The size D2 may be, for example, 200 μm or less, preferably 150 μm or less, 120 μm or less, or 100 μm or less, instead. The size D2 may be appropriately set by those skilled in the art in accordance with, for example, the size of the analyte. In a case where the analyte is a cell, for example, the size D2 may be, for example, from 1 μm to 200 μm, in particular from 10 μm to 100 μm. In a case where the analyte is a cellular constituent such as a nucleic acid or a protein, the size D2 may be, for example, from 50 nm to 1000 nm, in particular from 100 nm to 900 nm. The size D2 may mean, for example, a length of one side in a case where a shape of the bottom of the well is a square, a length of long sides in a case where the shape of the bottom is a rectangle, a length of the longest side in a case where the shape of the bottom is another rectangle, a longest diagonal distance in a case where the shape of the bottom is a polygon with five or more sides, a diameter in a case where the shape of the bottom is a circle, and a length of a major axis in a case where the shape of the bottom is an ellipse. The size D2 may be different from or the same as the size D1. In a case where the size D2 is different from the size D1, the size D1 of the opening may preferably be greater than the size D2 of the bottom as illustrated in the drawing, but the size D1 may be smaller than the size D2, instead.

Size D3 of depth of the well 103 may be, for example, 50 nm or more, preferably 100 nm or more, 200 nm or more, or 300 nm or more. The size D3 may be, for example, 200 μm or less, preferably 150 μm or less, 120 μm or less, or 100 μm or less, instead. The size D3 may be appropriately set by those skilled in the art in accordance with, for example, the size of the analyte. In a case where the analyte is a cell, for example, the size D3 may be, for example, from 1 μm to 200 μm, in particular from 10 μm to 100 μm. In a case where the analyte is a cellular constituent such as a nucleic acid or a protein, the size D3 may be, for example, from 50 nm to 1000 nm, in particular from 100 nm to 900 nm. The size D3 may mean a distance between the opening and the bottom.

In addition, size D4 (also referred to as a cell size) of the pixel unit 101 may be larger than the size D1 of the well, and may be, for example, 100 nm or more, preferably 200 nm or more, 300 nm or more, or 400 nm or more. The size D4 may be, for example, 300 μm or less, preferably 200 μm or less, 150 μm or less, or 100 μm or less, instead. The size D4 may be appropriately set by those skilled in the art in accordance with, for example, the size of the analyte. In a case where the analyte is a cell, for example, the size D4 may be, for example, from 2 μm to 400 μm, in particular from 10 μm to 200 μm. In a case where the analyte is a cellular constituent such as a nucleic acid or a protein, the size D4 may be, for example, from 100 nm to 5000 nm, in particular from 200 nm to 3000 nm. The size D4 may mean a length of one side in a case where a shape of the pixel unit is a square, a length of long sides in a case where the shape of the pixel unit is a rectangle, a length of the longest side in a case where the shape of the pixel unit is another rectangle, and a longest diagonal distance in a case where the shape of the pixel unit is a polygon with five or more sides.

The well 103 may be configured to hold the analyte. For example, a compound for holding an analyte may be immobilized on a surface (particularly a bottom surface) of the well 103. The compound may be appropriately selected by those skilled in the art in accordance with a type of analyte. The compound is, for example, a nucleic acid, but is not limited thereto, and may be another compound such as a protein, a peptide, a sugar, or a lipid, instead.

In a case where the analyte is a nucleic acid such as DNA or RNA, the compound may also be a nucleic acid such as DNA or RNA, but is not limited thereto, and may be, for example, a protein, a peptide, a sugar, or a lipid, instead.

The analyte may be a compound other than a nucleic acid or a bioparticle (for example, a cell or an endoplasmic reticulum). In such a case, the compound may be, but is not limited to, a nucleic acid, a protein, a peptide, a sugar, or a lipid.

The compound to be immobilized may be, for example, a compound for capturing a bioreceptor in the well (particularly the bottom surface of the well), and may be, for example, a SAM reagent, a bivalent reagent, an activation reagent (for example, a carboxylic acid activation reagent), or a biotinylation reagent.

The pixel unit 101 includes a photodiode 104. The photodiode may be a photodiode for detecting fluorescence. The photodiode may be, for example, a Si photodiode, and may have, for example, an N region 104N and a P region 104P of Si. As illustrated in the drawing, the N region 104N may be surrounded by the P region 104P.

The photodiode 104 is provided in such a way as to cover the side surface of the well in addition to the bottom of the well. The photodiode covering the side surface of the well is indicated in FIG. 2B by broken line regions indicated by a reference numeral W1 (in particular, regions of the reference numerals 104P and 104N). The photodiode covering the bottom surface of the well is indicated in the drawing by a broken line region indicated by a reference numeral W2 (in particular, regions of the reference numerals 104P and 104N). As described above, the photodiode 104 also has a well shape, and the photodiode 104 is configured to form side wall portions W1 and a bottom portion W2 of the well.

In the present disclosure, as described above, the photodiode may be configured to cover the bottom surface and the side surface of the well, that is, the photodiode also has a well shape. As a result, among the fluorescence generated by irradiating a sample S1 with excitation light L1, fluorescence traveling toward the well side surface is also detected in addition to fluorescence traveling toward the well bottom surface. This improves the fluorescence detection accuracy.

The pixel unit 101 includes a gate electrode unit 105 (also referred to as a TG). The gate electrode unit may include, for example, polysilicon (Poly-Si). In addition, the gate electrode unit may be configured as a vertical transfer gate (VG), instead, which will be described later. The pixel unit 101 further includes a floating diffusion FD to which electrons accumulated in the photodiode are transferred, and a contact CS connected to the floating diffusion.

As illustrated in the drawing, the photodiode 104 may have an embedded photodiode structure. The gate electrode unit 105 may be connected to the photodiode 104 having this structure. The electrons accumulated in the photodiode 104 are transferred from the gate electrode unit 105 to the floating diffusion FD, and then read from the contact CS.

In the present disclosure, a component provided in order to read electrons from the photodiode will also be referred to as an electronic reading unit. As described above, the electronic reading unit may include the gate electrode unit 105 (TG), the floating diffusion FD, and the contact CS. In the drawing, the floating diffusion is provided in each pixel unit, that is, the electronic reading unit has a structure of a so-called FD non-sharing type.

In the present disclosure, the electronic reading unit may have a structure in which a floating diffusion is shared by a plurality of pixel units, that is, may have a structure of a so-called FD sharing type. The number of pixel units sharing one FD may be, for example, four.

FIG. 2C illustrates a configuration example of the electronic reading unit of the FD non-sharing type and a configuration example of the electronic reading unit of the FD sharing type.
(a) of the drawing illustrates a schematic configuration example of an example of an electronic reading unit 251 of the FD sharing type (a portion surrounded by a broken line). The configuration example is the same as that in FIG. 2A. FIGS. 2C(b) and 2C(c) illustrate configuration examples of the electronic reading unit of the FD non-sharing type (portions surrounded by broken lines). Similarly to the electronic reading unit 250 illustrated in (a), an electronic reading unit 251 illustrated in (b) includes the gate electrode unit TG, the floating diffusion FD, and the contact CS, but FD and CS are provided at positions where FD and CS are shared with adjacent pixel units. FD and CS may be shared by, for example, four pixel units. In addition, as in an electronic reading unit 252 illustrated in (c), a Poly-Si contact may be used in an electronic reading unit of the FD-sharing type.

Note that, in the present disclosure, a shape, dimensions, and arrangement of the photodiode and the electronic reading unit may be appropriately changed by those skilled in the art, and are not limited to those described in these drawings.

The pixel unit 101 is separated from other unit pixels by partitions 106. The partitions 106 may also be referred to as trenches. As described above, in the back-illuminated imaging element in the present disclosure, trenches may be provided between the pixel units.

Each partition 106 is provided between a certain unit pixel and another unit pixel. The partitions 106 may include an insulator or a metal. The partitions 106 prevent the excitation light L1 that has entered the pixel unit 101 and the fluorescence generated from the analyte S1 in the pixel unit 101 from entering another unit pixel. In addition, the partitions 106 prevent electrons in the photodiode 104 from entering a photodiode of another unit pixel.

The imaging element 100 is of a back-illuminated type, that is, a wiring layer is provided on a side of the photodiode opposite a fluorescence incident side.

The well 103 is provided on one side of the photodiode 104, and the wiring layer is provided on an opposite side of the photodiode 104. That is, the pixel unit 101 has a multilayer structure in which the wiring layer, the detection unit (photodiode), and the analyte holding unit (well) are arranged in this order. With such a configuration, the imaging element in the present disclosure can obtain a larger fluorescence signal, which contributes to improvement of the fluorescence detection accuracy.

As illustrated in FIG. 1, the imaging element 100 may have a configuration in which the plurality of pixel units 101 is arranged in a lattice pattern. The number of pixel units 101 (that is, the number of pixels) included in one imaging element 100 may be appropriately selected by those skilled in the art in accordance with, for example, a factor such as the size of the imaging element or an imaging target. The number of pixels may be, for example, 500 pixels or more, and may be particularly 1,000 pixels or more, 5,000 pixels or more, 10,000 pixels or more, 50,000 pixels or more, or 100,000 pixels or more. An upper limit value of the number of pixels of the imaging element is not necessarily specified, but may be, for example, 10 million pixels or less, and may be particularly 8 million pixels or less, 6 million pixels or less, 4 million pixels or less, or 2 million pixels or less.

A lower limit value of the size of the imaging element 100 may be, for example, 3 mm or more, and may be particularly 5 mm or more, 7 mm or more, or 10 mm or more. An upper limit value of the size may be, for example, 80 mm or less, and particularly 70 mm or less or 60 mm or less. In one implementation, the imaging element may have a size of, for example, 3 mm to 80 mm (size of one side of a rectangle)×3 mm to 80 mm (size of another side of the rectangle), and may particularly have a size of 10 mm to 60 mm×10 mm to 60 mm. The size of the imaging element may mean size of the light receiving surface on which the pixel units are arranged.

A shape of the imaging element may be, for example, a rectangle, and more specifically, an oblong or a square. In a case where the shape of the light receiving surface of the imaging element is an oblong, the size of the imaging element may mean long sides of the light receiving surface (short sides may be shorter than the size). In a case where the shape of the light receiving surface of the imaging element is a square, the size of the imaging element may mean one side of the light receiving surface.

In a case where the imaging element is incorporated into a biological sample analysis system, only one imaging element may be incorporated, or two or more imaging elements may be incorporated. For example, in a case where two or more imaging elements are used, these imaging elements may be arranged, for example, in a tile pattern. For example, the biological sample analysis system may include a plurality of imaging elements according to the present disclosure, and the plurality of imaging elements may be joined together by tiling. The plurality of imaging elements joined together may be used as one sensor, and in particular, may form one imaging surface. The plurality of imaging elements may include one type of imaging element, or may include two or more types of imaging elements.

In a case where the imaging element 100 is used to analyze a biological sample, a space may be formed in order to cause an analyte to reach and be held in the well. The space may be a flow channel through which an analyte flows. For example, a liquid sample containing an analyte may flow through the space, and the analyte may be captured at the bottom surface of the well. At least a part of the space may be formed in a well shape. Another part of the space may be formed by a transparent substrate 108 as illustrated in FIG. 1. Since the transparent substrate 108 is transparent, excitation light can reach an inside of the well. A material of the transparent substrate 108 may be, for example, glass, but may be a resin (for example, acrylic resin, polycarbonate resin, etc.).

The imaging element 100 may thus have a space for allowing an analyte to reach the inside of the well, and may further include the transparent substrate 108 that forms the space.

### 1.1.2 Example 1-2 (Wells with Column Structure)

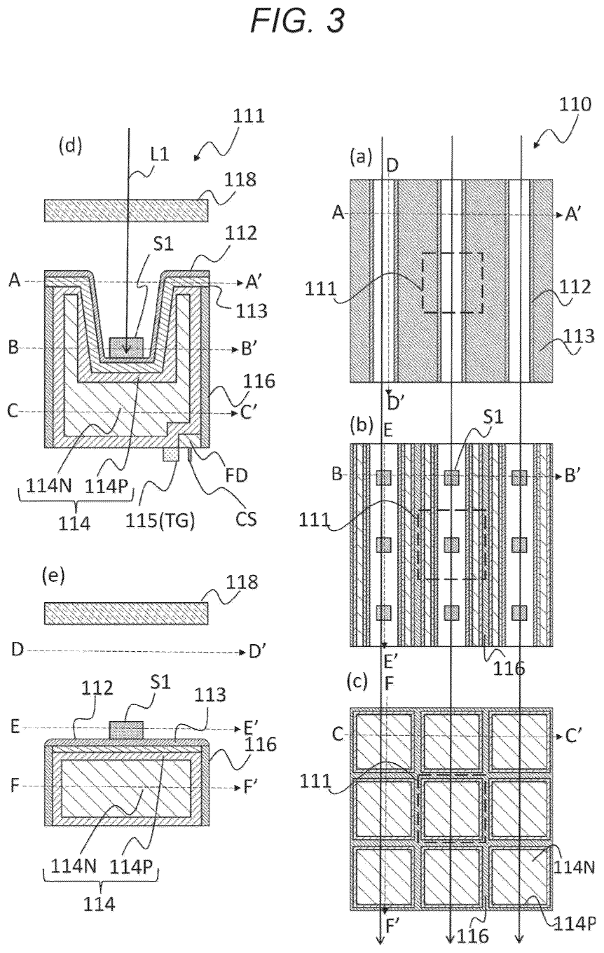

The imaging element 100 described in Example 1-1 described above is used such that the excitation light L1 perpendicularly enters the light receiving surface of the imaging element. In the present disclosure, excitation light traveling in a direction parallel to the light receiving surface may be applied to an analyte held in the well. For the radiation, two or more wells may be connected to each other in such a way as to form a column structure. An example of an imaging element onto which such excitation light is radiated will be described with reference to FIG. 3. The drawing is a schematic diagram of a structure of the back-illuminated imaging element according to the present disclosure.
(d) on a left side of the drawing is a schematic diagram of a cross section of a pixel unit 111 of the imaging element. The cross section is a cross section in a plane perpendicular to a light receiving surface of the imaging element.
(a) to (c) on a right side of the drawing are schematic diagrams illustrating a part of the light receiving surface of an imaging element 110 in which pixel units 111 are arranged in a lattice pattern, and more specifically, are schematic diagrams of cross sections as follows.
(a) on the right side of the drawing is a schematic diagram of a cross section at a position of broken line A-A′ in (d) of the drawing, and this schematic diagram illustrates a state in which the plurality of pixel units is arranged in a lattice pattern. In addition, the cross section is a cross section in a plane parallel to the light receiving surface of the imaging element.
(b) on the right side of the drawing is a schematic diagram of a cross section at a position of broken line B-B′ in (d) of the drawing, and this schematic diagram illustrates a state in which the plurality of pixel units is arranged in a lattice pattern. In addition, the cross section is a cross section in a plane parallel to the light receiving surface of the imaging element.
(c) on the right side of the drawing is a schematic diagram of a cross section at a position of broken line C-C′ in (d) of the drawing, and this schematic diagram illustrates a state in which the plurality of pixel units is arranged in a lattice pattern. In addition, the cross section is a cross section in a plane parallel to the light receiving surface of the imaging element.
(e) on the left side of the drawing is a schematic diagram of a cross section of the pixel unit 111 of the imaging element. The cross section is a cross section in a plane perpendicular to the light receiving surface of the imaging element and parallel to a direction in which the excitation light L1 travels. Broken line D-D′ in (e) corresponds to broken line D-D′ in (a) on the right side of the drawing. Broken line E-E′ in (e) corresponds to broken line E-E′ in (a) on the right side of the drawing. Broken line F-F′ in (e) corresponds to broken line F-F′ in (a) on the right side of the drawing.

The pixel unit 111 includes a well 113. As illustrated in the drawing, the well 113 may be covered with an insulating film 112.

The well 113 of the pixel unit 111 has a linear shape. The well 113 forms one line together with wells of two adjacent pixel units. The excitation light L1 is radiated in such a way as to travel along a direction of this line.

The well 113 (and the insulating film 112 covering the well) may be configured to hold an analyte, that is, corresponds to the analyte holding unit.

Since the wells of the plurality of pixel units form a line in this manner, the excitation light L1 can travel in parallel with the column. As a result, the excitation light L1 can be prevented from entering the photodiode 114. As a result, noise caused by the excitation light L1 can be reduced.

Size of the well 113 and the pixel unit 111 may be similar to that in Example 1-1 described above, and the description also applies to the present example.

Note that width of the line in an opening of the well 113 corresponds to the size D1 of the opening of the well 103. In addition, width of the line on a bottom surface of the well 113 corresponds to the size D2 of a bottom of the well 103.

The well 113 may be configured to hold an analyte as with the well 103 described in Example 1-1 above. That is, configuration of a surface of the well 113 may be similar to the configuration of the surface of the well 103.

The pixel unit 111 includes a photodiode 114. The photodiode may be, for example, a Si photodiode, and may have, for example, an N region 114N and a P region 115P of Si. As illustrated in the drawing, the N region 114N may be surrounded by the P region 115P.

As with the photodiode 104 described in Example 1-1 above, the photodiode 114 is provided in such a way as to cover a side surface of the well in addition to the bottom of the well.

In the present disclosure, as described above, the photodiode may be configured to cover the bottom surface and the side surface of the well, that is, the photodiode also has a well shape. As a result, among the fluorescence generated by irradiating a sample S1 with excitation light L1, fluorescence traveling toward the well side surface is also detected in addition to fluorescence traveling toward the well bottom surface. This improves the fluorescence detection accuracy.

The pixel unit 111 includes polysilicon (Poly-Si) 115 (also referred to as a TG). The polysilicon functions as a gate electrode unit. The pixel unit 111 further includes a floating diffusion FD to which electrons accumulated in the photodiode are transferred, and a contact CS connected to the floating diffusion. These TG, FD, and CS will also be referred to as an electronic reading unit as described in Example 1-1 above. The electronic reading unit and the TG, FD, and CS are as described in Example 1-1 above, and the description also applies to the present example.

The pixel unit 111 is separated from other unit pixels by partitions 116. The partitions 116 may also be referred to as trenches. The partitions 116 may be configured in the same manner as the partitions 106 described in Example 1-1 above.

The imaging element 110 is of a back-illuminated type, that is, a wiring layer is provided on a side of the photodiode opposite a fluorescence incident side.

The well 113 is provided on one side of the photodiode 114, and the wiring layer is provided on an opposite side of the photodiode 114. That is, the pixel unit 111 has a multilayer structure in which the wiring layer, the detection unit (photodiode), and the analyte holding unit (well) are arranged in this order. With such a configuration, the imaging element in the present disclosure can obtain a larger fluorescence signal, which contributes to improvement of the fluorescence detection accuracy.

The number of pixel units included in the imaging element 110 and the size of the imaging element 110 may be as described for the imaging element 100 in Example 1 above, and the description also applies to the present example.

In a case where the imaging element 110 is used to analyze a biological sample, a space may be formed in order to cause an analyte to reach and be held in the well. At least a part of the space may be formed in a well shape. Another part of the space may be formed by a transparent substrate 118 as illustrated in FIG. 3. Since the transparent substrate 118 is transparent, excitation light can reach an inside of the well. A material of the transparent substrate 118 may be, for example, glass, but may be a resin (for example, acrylic resin, polycarbonate resin, etc.). The imaging element 110 may thus have a space for allowing an analyte to reach the inside of the well, and may further include the transparent substrate 118 that forms the space.

### 1.1.3 Example 1-3 (Control of Position of Analyte by Electrodes)

The imaging element according to the present disclosure may be provided with an electrode pair in order to control a position where an analyte is held in the column structure described in Example 1-2 above. The electrode pair may be a pair of a first electrode and a second electrode. Each of the first electrode and the second electrode constituting the electrode pair may be preferably a transparent electrode layer, or may be a metal electrode layer.

The first electrode and/or the second electrode may be preferably a transparent electrode layer. The transparent electrode layer can prevent a decrease in light (excitation light and/or fluorescence).

In one implementation, an insulating film may be stacked on the transparent electrode layer or the metal electrode layer. In this implementation, the electrode layer and the insulating film may be configured to transmit an electric field to an analyte through capacitance coupling. The position of the analyte (biologically derived material) may thus be controlled.

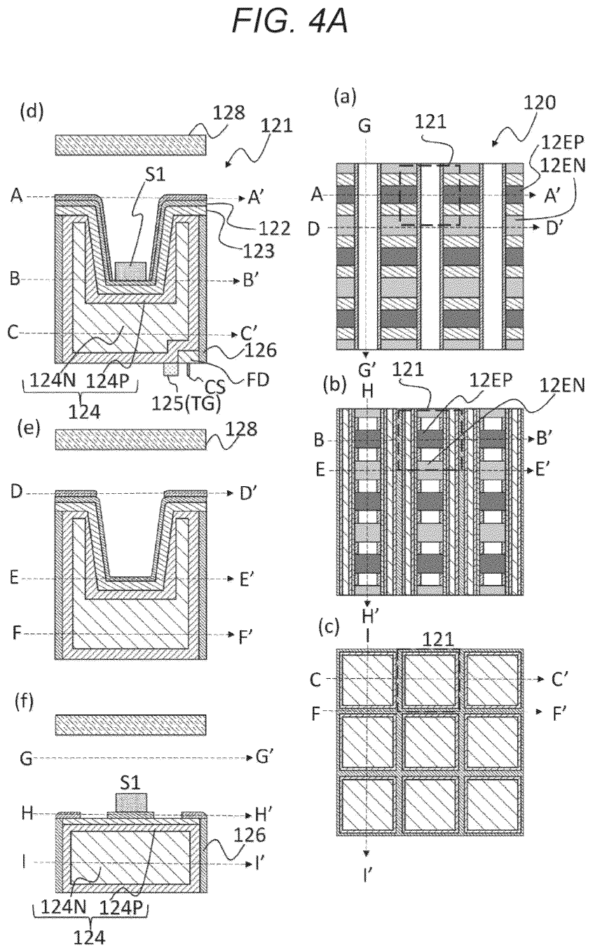

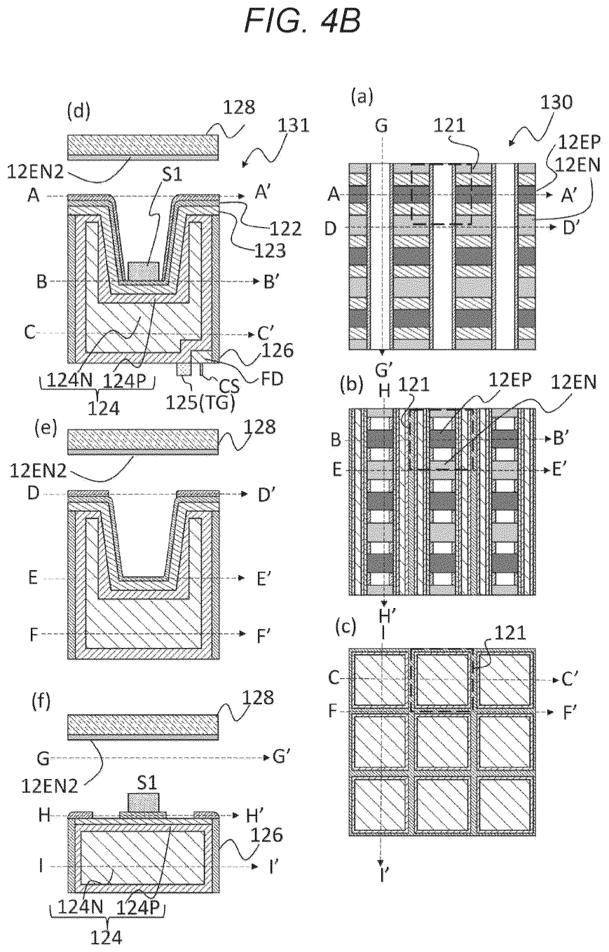

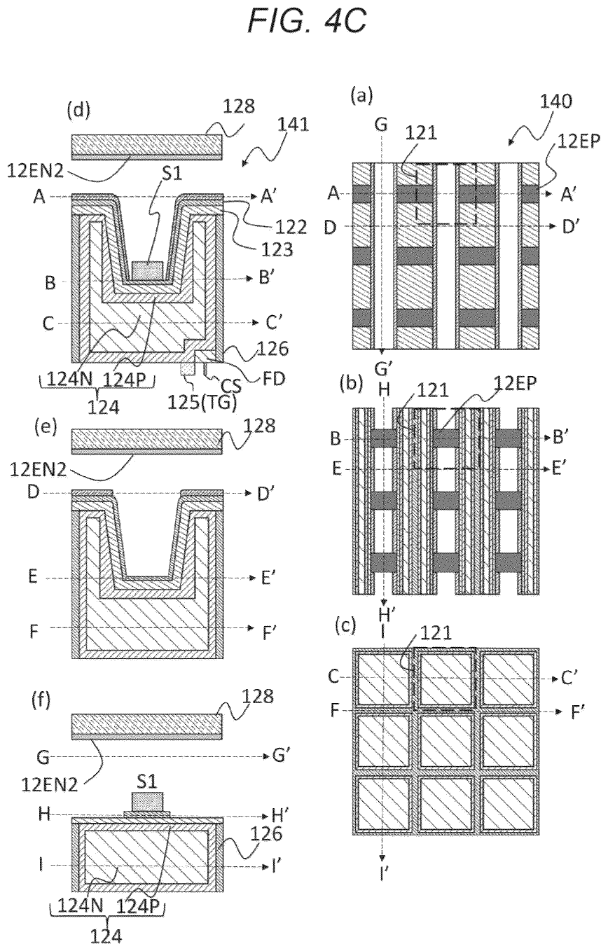

Configuration examples of the imaging element provided with the electrode pair will be described with reference to FIGS. 4A to 4C. Each of the imaging elements illustrated in these drawings has the same configuration as the imaging element described in Example 1-2 above except that an electrode pair for holding an analyte at a predetermined position is added to the configuration of the imaging element described in Example 1-2 above. For this reason, configuration of the electrode pair will be mainly described below.

FIG. 4A illustrates an example of a configuration in which both the first electrode and the second electrode constituting the electrode pair are provided on the well.
(d) on a left side of the drawing is a schematic diagram of a cross section of a pixel unit 121 of the imaging element. The cross section is a cross section in a plane perpendicular to a light receiving surface of the imaging element.
(a) to (c) on a right side of the drawing are schematic diagrams illustrating a part of the light receiving surface of an imaging element 120 in which pixel units 121 are arranged in a lattice pattern, and more specifically, are schematic diagrams of cross sections as follows.
(a) on the right side of the drawing is a schematic diagram of a cross section at a position of broken line A-A′ in (d) of the drawing, and this schematic diagram illustrates a state in which the plurality of pixel units is arranged in a lattice pattern. In addition, the cross section is a cross section in a plane parallel to the light receiving surface of the imaging element.
(b) on the right side of the drawing is a schematic diagram of a cross section at a position of broken line B-B′ in (d) of the drawing, and this schematic diagram illustrates a state in which the plurality of pixel units is arranged in a lattice pattern. In addition, the cross section is a cross section in a plane parallel to the light receiving surface of the imaging element.
(c) on the right side of the drawing is a schematic diagram of a cross section at a position of broken line C-C′ in (d) of the drawing, and this schematic diagram illustrates a state in which the plurality of pixel units is arranged in a lattice pattern. In addition, the cross section is a cross section in a plane parallel to the light receiving surface of the imaging element.
(e) on the left side of the drawing is a schematic diagram of a cross section of the pixel unit 121 of the imaging element. The cross section is a cross section in a plane perpendicular to the light receiving surface of the imaging element and parallel to a direction in which the excitation light L1 travels. Broken line D-D′ in (e) corresponds to broken line D-D′ in (a) on the right side of the drawing. Broken line E-E′ in (e) corresponds to broken line E-E′ in (b) on the right side of the drawing. Broken line F-F′ in (e) corresponds to broken line F-F′ in (c) on the right side of the drawing.
(f) on the left side of the drawing is a schematic diagram of a cross section of the pixel unit 121 of the imaging element. The cross section is a cross section in a plane perpendicular to the light receiving surface of the imaging element and parallel to a direction in which the excitation light L1 travels. Broken line G-G′ in (f) corresponds to broken line G-G′ in (a) on the right side of the drawing. Broken line H-H′ in (f) corresponds to broken line H-H′ in (b) on the right side of the drawing. Broken line I-I′ in (f) corresponds to broken line I-I′ in (c) on the right side of the drawing.

The pixel unit 121 includes a first electrode 12EP and a second electrode 12EN. The first electrode 12EP and the second electrode 12EN form a complementary electrode pair.

The first electrode 12EP is present immediately below the position where the analyte S1 is to be held, that is, provided in such a way as to pass through the center of the pixel unit. The first electrode 12EP may be provided in such a way as to cross the column structure.

The second electrode 12EN is provided in such a way as to pass between the pixels, particularly in such a way as to pass through a boundary between the pixel units.

Here, for example, it is assumed that the first electrode 12EP is a positive electrode, the second electrode 12EN is a negative electrode, and the analyte is DNA. Since DNA has a negative charge, a force acts on the DNA such that the DNA approaches the first electrode 12EP and separates from the second electrode EN by applying a voltage between these two electrodes. As a result, the DNA is maintained at the center of the pixel unit.

Note that the first electrode 12EP may be a negative electrode, and the second electrode 12 EN may be a positive electrode, instead. Polarity of these electrodes may be appropriately changed in accordance with a type of analyte or a control method.

In addition, the voltage may be a DC voltage or an AC voltage.

FIG. 4B illustrates an example of a configuration in which the first electrode constituting the electrode pair is provided on the well and two second electrodes are provided on the well and the transparent substrate.

An imaging element 130 and a pixel unit 131 illustrated in the drawing are the same as the imaging element 120 and the pixel unit 121 described with reference to FIG. 4A except that a second electrode 12EN2 is stacked on a transparent substrate 128.

As described with reference to FIG. 4A, it is assumed that the first electrode 12EP is a positive electrode, the second electrodes 12EN and 12EN2 are negative electrodes, and the analyte is DNA.

Since DNA has a negative charge, a force acts on the DNA such that the DNA approaches the first electrode 12EP and separates from the second electrode EN by applying a voltage between the first electrode 12EP and the second electrode 12EN. As a result, the DNA is maintained at the center of the pixel unit.

Furthermore, a force acts on the DNA such that the DNA approaches the first electrode 12EP and separates from the second electrode EN2 by applying a voltage between the first electrode 12EP and the second electrode 12EN2. As a result, the DNA is maintained in such a way as to be pressed against the bottom surface of the well.

As described above, since the force to maintain the DNA at the center of the pixel unit and the force to press the DNA against the bottom surface of the well act on the DNA, the DNA is more reliably maintained at a desired position.

In one implementation, the second electrode 12EN may be omitted, that is, the imaging element 130 may be configured to not include the second electrode 12EN and include the first electrode 12EP and the second electrode 12EN2. With this configuration, too, the analyte can be controlled.

In addition, although the second electrode 12EN is a negative electrode in the above configuration example, the second electrode 12EN may be configured as a positive electrode, instead. That is, the imaging element 130 may include two first electrodes 12EP and 12EN (also referred to as 12EP2) as positive electrodes and one second electrode 12EN as a negative electrode.

As described above, in the present disclosure, the first electrode and the second electrode may be configured to be capable of controlling the position of the analyte by applying a voltage (DC voltage or AC voltage). The number of electrodes included in the first electrode may be one, or two or more. In addition, the number of electrodes included in the second electrode may be one, or two or more. The number, shape, and position of the first electrode and the second electrode may be appropriately changed by those skilled in the art.

FIG. 4C illustrates an example of a configuration in which the first electrode constituting the electrode pair is provided on the well and two second electrodes are provided on the transparent substrate.

An imaging element 140 and a pixel unit 141 illustrated in the drawing are the same as the imaging element 130 and the pixel unit 131 described with reference to FIG. 4B except that a second electrode 12EN2 is stacked on a transparent substrate 128 and a second electrode is not provided on the well.

As described with reference to FIG. 4B, it is assumed that the first electrode 12EP is a positive electrode, 12EN2 is a negative electrode, and the analyte is DNA.

DNA has a negative charge. A force, therefore, acts on the DNA such that the DNA approaches the first electrode 12EP and separates from the second electrode EN2 by applying a voltage between the first electrode 12EP and the second electrode 12EN2. As a result, the DNA is maintained in such a way as to be pressed against the bottom surface of the well. As a result, the DNA is maintained in such a way as not to be separated from the bottom surface of the well.

### 1.1.4 Example 1-4 (Structure without Trenches)

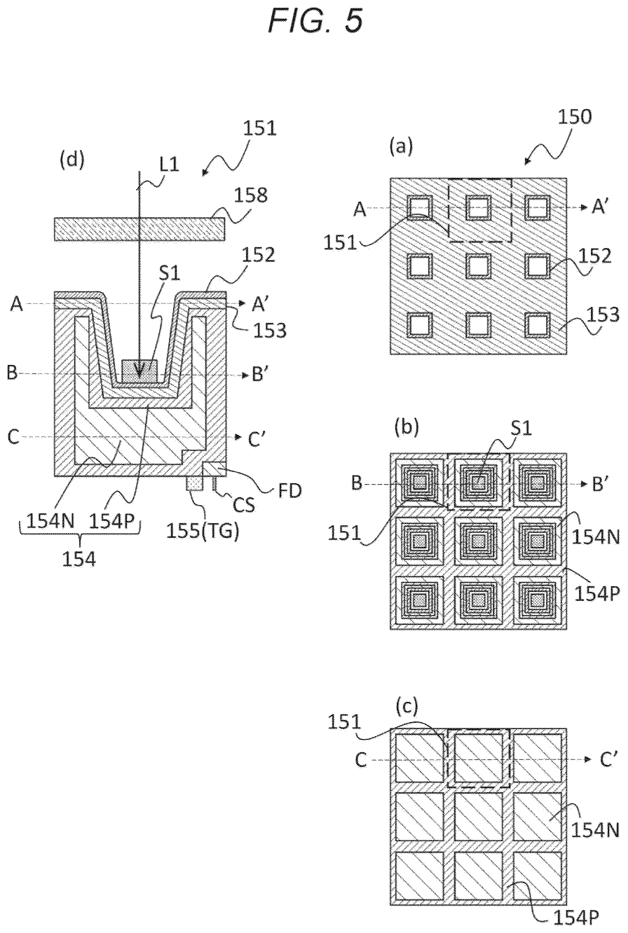

Among the components of the imaging element described in Example 1-1 above, the partitions may be omitted. An example of a configuration in which the partitions are omitted will be described with reference to FIG. 5.
(d) on a left side of the drawing is a schematic diagram of a cross section of a pixel unit 151 of an imaging element 150. The cross section is a cross section in a plane perpendicular to a light receiving surface of the imaging element.
(a) on the right side of the drawing is a schematic diagram of a cross section at a position of broken line A-A′ in (d) of the drawing, and this schematic diagram illustrates a state in which the plurality of pixel units is arranged in a lattice pattern. In addition, the cross section is a cross section in a plane parallel to the light receiving surface of the imaging element.
(b) on the right side of the drawing is a schematic diagram of a cross section at a position of broken line B-B′ in (d) of the drawing, and this schematic diagram illustrates a state in which the plurality of pixel units is arranged in a lattice pattern. In addition, the cross section is a cross section in a plane parallel to the light receiving surface of the imaging element.
(c) on the right side of the drawing is a schematic diagram of a cross section at a position of broken line C-C′ in (d) of the drawing, and this schematic diagram illustrates a state in which the plurality of pixel units is arranged in a lattice pattern. In addition, the cross section is a cross section in a plane parallel to the light receiving surface of the imaging element.

The pixel unit 151 is the same as the pixel unit 101 except that the partitions 106 are not provided.

That is, an insulating film 152, a well 153, a photodiode 154 (154N and 154P), a gate electrode unit 155 (TG), a floating diffusion FD, and a contact CS constituting the pixel unit 151 may be similar to the insulating film 102, the well 103, the photodiode 104 (104N and 104P), the gate electrode unit 105, the floating diffusion FD, and the contact CS described in Example 1-1 above, and the description of these also applies to the present example.

The imaging element 150 without the partitions can reduce a manufacturing cost as compared with the imaging element 100 described in Example 1-1 above. In addition, in a case where a fluorescence signal is too weak, fluorescence signals of adjacent pixels can be added.

### 1.1.5 Example 1-5 (Use of Multilayer Film Reflection Filter)

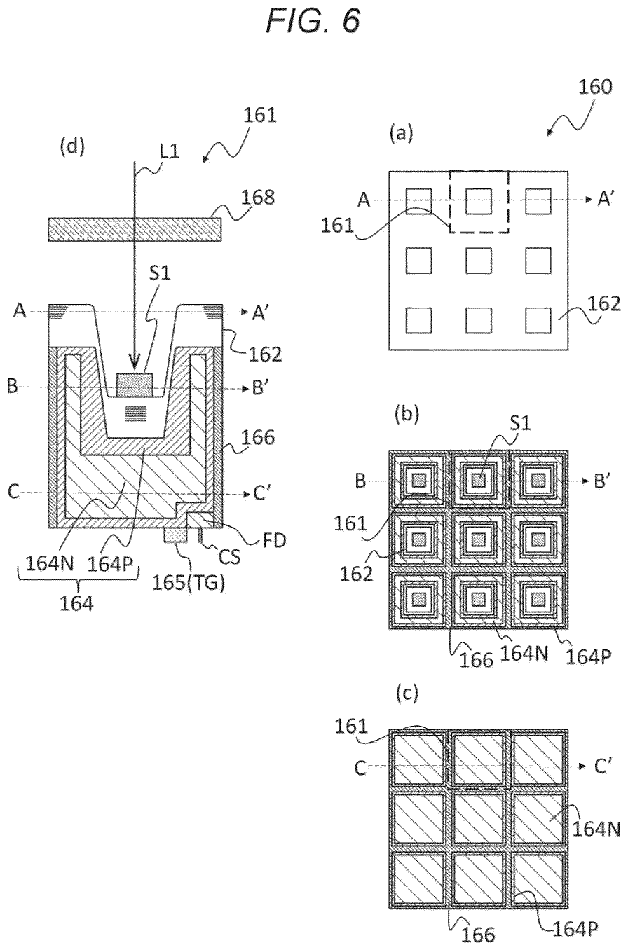

A well surface of the imaging element described in Example 1-1 above may be formed by a multilayer film reflection filter. A configuration example of an imaging element including a multilayer film reflection filter will be described with reference to FIG. 6.
(d) on a left side of the drawing is a schematic diagram of a cross section of a pixel unit 161 of an imaging element 160. The cross section is a cross section in a plane perpendicular to a light receiving surface of the imaging element.
(a) on the right side of the drawing is a schematic diagram of a cross section at a position of broken line A-A′ in (d) of the drawing, and this schematic diagram illustrates a state in which the plurality of pixel units is arranged in a lattice pattern. In addition, the cross section is a cross section in a plane parallel to the light receiving surface of the imaging element.
(b) on the right side of the drawing is a schematic diagram of a cross section at a position of broken line B-B′ in (d) of the drawing, and this schematic diagram illustrates a state in which the plurality of pixel units is arranged in a lattice pattern. In addition, the cross section is a cross section in a plane parallel to the light receiving surface of the imaging element.
(c) on the right side of the drawing is a schematic diagram of a cross section at a position of broken line C-C′ in (d) of the drawing, and this schematic diagram illustrates a state in which the plurality of pixel units is arranged in a lattice pattern. In addition, the cross section is a cross section in a plane parallel to the light receiving surface of the imaging element.

The pixel unit 161 is the same as the pixel unit 101 except that a multilayer film reflection filter 162 is used instead of the well 103 and the insulating film 102.

That is, a photodiode 164 (164N and 164P), a gate electrode unit 165 (TG), a floating diffusion FD, a contact CS, and a transparent substrate 168 constituting the pixel unit 161 may be similar to the photodiode 104 (104N and 104P), the gate electrode unit 105 (TG), the floating diffusion FD, the contact CS, and the transparent substrate 108 described in Example 1-1 above, and the description of these also applies to the present example.

Since a well surface is formed by the multilayer film reflection filter 162, it is possible to prevent the excitation light L1 from entering the photodiode 164. As a result, noise caused by the excitation light L1 can be reduced, and the fluorescence detection accuracy can be improved.

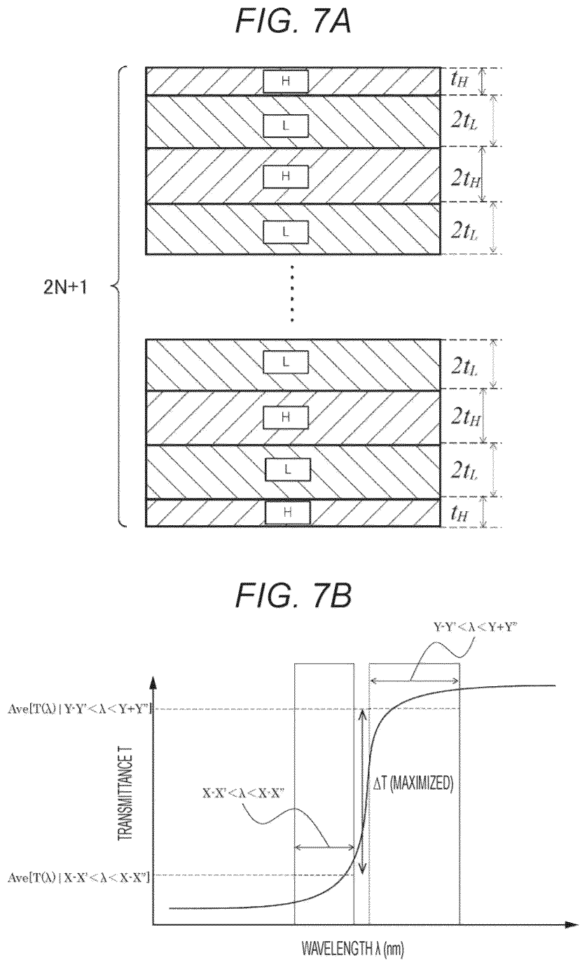

A configuration example of the multilayer film reflection filter will be described with reference to FIGS. 7A and 7B.

FIG. 7A is a schematic diagram of a multilayer structure of the filter. As illustrated in the drawing, the multilayer film reflection filter has a multilayer structure in which a layer H including a high refractive index material (hereinafter also referred to as a “high refractive index layer H”) and a layer L including a low refractive index material (hereinafter also referred to as a “low refractive index layer H”) are alternately stacked.

As illustrated in the drawing, both of two outermost layers of the multilayer film reflection filter may be, for example, a high refractive material layer H having a thickness t_{H}. As illustrated in the drawing, a low refractive index material layer L having a thickness of 2t_{L} and a high refractive index material layer H having a thickness of 2t_{H} may be alternately stacked between these two layers. Thickness of each layer may be on the order of nm.

A difference ΔT between an “average transmittance in a wavelength range of excitation light intended to be blocked by the multilayer film reflection filter” and an “average transmittance in a wavelength range of fluorescence intended to pass through the multilayer film reflection filter” is expressed by the following Expression (I). In the present disclosure, a dielectric multilayer film may be configured such that, for example, the difference ΔT is 99% or more, and is particularly configured to maximize the difference ΔT. Transmittance T in Expression (I) can be calculated by a method known in the art, and a calculation method will be described later.

$$[ Math . 1 ]$$

$$Δ T = ave [ T ( λ ) \vert X - {X}^{′} < λ < X + {X}^{″} ] \cdot ave [ T ( λ ) \vert Y - {Y}^{′} < λ < Y + {Y}^{″} ] \qquad ( 1 )$$

Assumptions of the above Expression (I) are as follows.

Excitation light: X nm (consideration range: X−X′<λ<X−X″)

(The consideration range corresponds to the wavelength range of the excitation light intended to be blocked.)

Fluorescence: Y nm (consideration range: Y−Y′<λ<Y+Y″)

(The consideration range corresponds to the wavelength range of the fluorescence intended to pass.)

High refractive index material: refractive index NH, film thickness t_{H}

Low refractive index material: refractive index NL, film thickness t_{L}

Number of repetitions: N (N is an integer larger than 1)

Total number of layers included in the multilayer film reflection filter: L=2N+1 layers (L is an integer larger than 3) Total thickness of the multilayer film reflection filter: 2N (t_{H}+t_{L}) nm

Maximization of ΔT is described on the basis of, for example, a schematic graph of transmittance with respect to wavelength in FIG. 7B. As illustrated in the drawing, the multilayer film reflection filter is configured such that the difference ΔT between an average transmittance Ave [T(λ) Y−Y′<λ<Y+Y″] in a range where the wavelength λ is from Y−Y′ to Y+Y″ and Ave [T(λ)|X−X′<λ<X+X″] in a range where the wavelength λ is from X−X′ to X+X″ is maximized.

### (Method for Calculating Transmittance T in Expression (I))

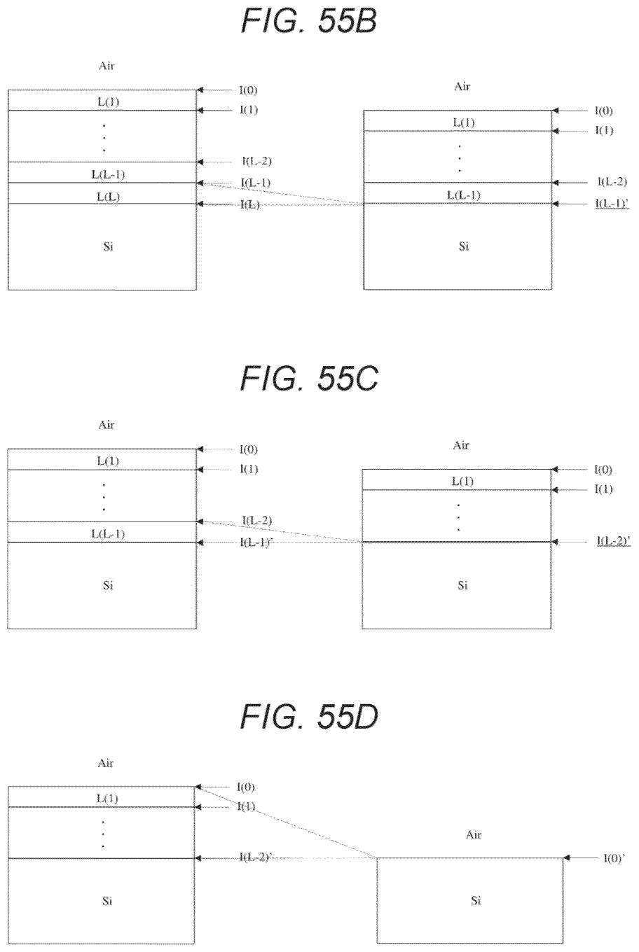

FIG. 55A is a schematic diagram illustrating a situation in which light passes through the multilayer film reflection filter. As illustrated in the drawing, in this situation, light incident perpendicularly to an in-plane direction of the multilayer film 162 from air (Air, refractive index n₀=1) passes through the multilayer film of L layers (complex refractive index of a j-th layer: Nⱼ=nⱼ+ikⱼ), and is emitted to the photodiode 164 (Si, complex refractive index: Nₘ=nₘ+ikₘ).

In addition to an interface I(0) between the multilayer film 162 and air and an interface I(L) with the photodiode 164 between the multilayer film 162 and air, there are interfaces I(1) to I(L−1) between the layers constituting the multilayer film 162.

In a case where the material of the multilayer film (that is, the complex refractive index) is determined, a Fresnel coefficient of reflection at each interface, a Fresnel coefficient of transmission at each interface, and a phase change and wave attenuation in each layer are determined. The transmittance T can be determined in accordance with a calculation method described on pages 99 to 103 of a document “Basic Theory of Optical Thin Film: Augmented and Revised Edition” (The Optronics Co., Ltd., issued on Feb. 25, 2011) using these values determined on the basis of the material of the multilayer film. More specifically, the transmittance T is obtained using the following Expression (II) in the calculation method.

$$[ Math . 2 ]$$

$$T = \frac{Re ( {N}_{m} )}{{n}_{0}} {\vert {τ}_{0}^{′} \vert}^{2} \qquad ( II )$$

The above Expression (II) is described in the document on page 103 (particularly Expression (4-50))

Elements in the above Expression (II) are as follows.

Re(Nₘ) is a real part (=nₘ) of the complex refractive index of Si as described above.

n₀ is the refractive index of air (=1) as described above.

τ₀′ is a Fresnel coefficient of transmission at a virtual interface I(0)′ described later, and is as described in Expression (4-48) on page 101 of the document. A method for calculating τ₀′ is described on pages 99 to 103 of the document.

In the calculation method, a Fresnel coefficient of the virtual interface I(L−1)′ is obtained from the Fresnel coefficients of the interface I(L) and the interface I(L−1) as illustrated in FIG. 55B, a Fresnel coefficient of the virtual interface I(L−2)′ is obtained from the Fresnel coefficient of the virtual interface I(L−1) and the Fresnel coefficient of the interface I(L−2) as illustrated in FIG. 55C, and the Fresnel coefficient of the virtual interface I(0)′ is obtained by repeating similar calculation of Fresnel coefficients of virtual interfaces as illustrated in FIG. 55D.

### 1.1.6 Example 1-6 (Multilayer Film Reflection Filter and Trenches Extended to Filter)

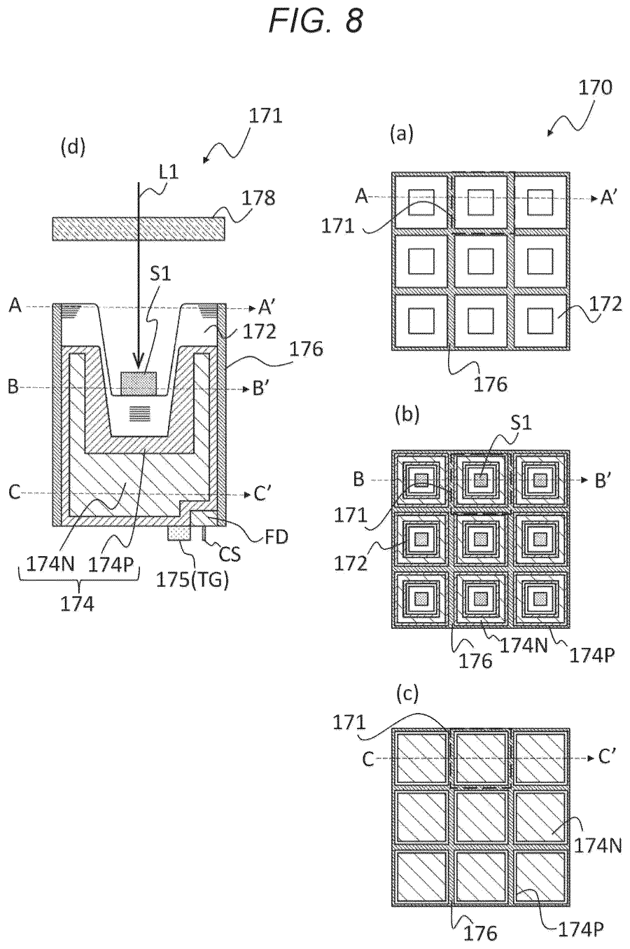

A well surface of the imaging element described in Example 6 above is formed by a multilayer film reflection filter. In the present disclosure, the multilayer film reflection filter may be divided for each pixel by partitions. That is, the partitions defining pixel units may be extended not only to the photodiode but also to the multilayer film reflection filter. This configuration will be described with reference to FIG. 8.
(d) on a left side of the drawing is a schematic diagram of a cross section of a pixel unit 171 of an imaging element 170. The cross section is a cross section in a plane perpendicular to a light receiving surface of the imaging element.
(a) on the right side of the drawing is a schematic diagram of a cross section at a position of broken line A-A′ in (d) of the drawing, and this schematic diagram illustrates a state in which the plurality of pixel units is arranged in a lattice pattern. In addition, the cross section is a cross section in a plane parallel to the light receiving surface of the imaging element.
(b) on the right side of the drawing is a schematic diagram of a cross section at a position of broken line B-B′ in (d) of the drawing, and this schematic diagram illustrates a state in which the plurality of pixel units is arranged in a lattice pattern. In addition, the cross section is a cross section in a plane parallel to the light receiving surface of the imaging element.
(c) on the right side of the drawing is a schematic diagram of a cross section at a position of broken line C-C′ in (d) of the drawing, and this schematic diagram illustrates a state in which the plurality of pixel units is arranged in a lattice pattern. In addition, the cross section is a cross section in a plane parallel to the light receiving surface of the imaging element.

In the pixel unit 161 described in Example 5 above, partitions 166 extend to a position of the photodiode 164, but in the pixel unit 171 in the present example, partitions 176 extend to a multilayer film reflection filter 172. By forming the partitions 176 in this manner, it is possible to prevent the excitation light L1 from entering other pixels, which contributes to reduction of noise due to the excitation light. The fluorescence detection accuracy, therefore, can be further improved.

### 1.1.7 Example 1-7 (Variations of Multilayer Film Reflection Filter and Trenches)

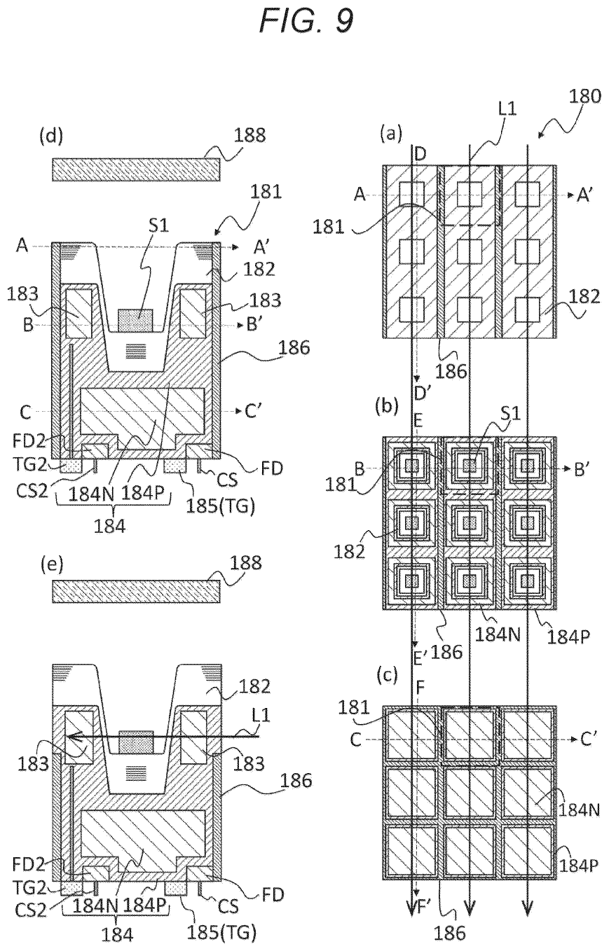

In the imaging element described in Example 6 above, the partitions are extended to the multilayer film reflection filter over the entire periphery of the pixel unit (all four sides of a rectangle defining the pixel unit). In the present disclosure, the partitions may extend to the multilayer film reflection filter in a part of the periphery of the pixel unit, and the partitions need not extend to the multilayer film reflection filter in the rest of the periphery of the pixel unit and may exist up to the photodiode for detecting fluorescence. This configuration will be described with reference to FIG. 9.
(d) on a left side of the drawing is a schematic diagram of a cross section of a pixel unit 181 of an imaging element 180. The cross section is a cross section in a plane perpendicular to a light receiving surface of the imaging element.
(a) on the right side of the drawing is a schematic diagram of a cross section at a position of broken line A-A′ in (d) of the drawing, and this schematic diagram illustrates a state in which the plurality of pixel units is arranged in a lattice pattern. In addition, the cross section is a cross section in a plane parallel to the light receiving surface of the imaging element.
(b) on the right side of the drawing is a schematic diagram of a cross section at a position of broken line B-B′ in (d) of the drawing, and this schematic diagram illustrates a state in which the plurality of pixel units is arranged in a lattice pattern. In addition, the cross section is a cross section in a plane parallel to the light receiving surface of the imaging element.
(c) on the right side of the drawing is a schematic diagram of a cross section at a position of broken line C-C′ in (d) of the drawing, and this schematic diagram illustrates a state in which the plurality of pixel units is arranged in a lattice pattern. In addition, the cross section is a cross section in a plane parallel to the light receiving surface of the imaging element.
(e) on the left side of the drawing is a schematic diagram of cross sections taken along broken line D-D′, broken line E-E′, and broken line F-F′ in (a), (b), and (c) of the drawing. The cross section is a cross section in a plane perpendicular to a light receiving surface of the imaging element.

In the cross section illustrated in (d) of the drawing, partitions 186 are extended from a photodiode 184 to a multilayer film reflection filter 182.

In the cross section illustrated in (e) of the drawing, on the other hand, the partitions 186 are provided up to the photodiode 184, but are not provided on the multilayer film reflection filter 182. As a result, the excitation light L1 in (a), (b), and (c) in the drawing can be emitted in such a way as to travel in a direction of arrows. In a case where the excitation light L1 is emitted in such a way as to travel in this direction, the excitation light can be prevented from traveling to the photodiode 184, and noise caused by the excitation light can be reduced. This contributes to improvement of the fluorescence detection accuracy.

In addition, as illustrated in the drawing, N regions 183 for detecting the excitation light L1 may be provided in side wall portions of the well. This makes it possible to read a signal of the excitation light.

In addition, as illustrated in the drawing, the N regions 183 may be connected to the trenches 186. As a result, carriers (electrons or holes) can be read.

In this implementation, in addition to the fluorescence signal, an excitation light signal is also read. Accordingly, the pixel unit 181 may include two sets of electronic reading units, one of which reads the fluorescence signal and the other reads the excitation light signal.

As illustrated in the drawing, the pixel unit 181 includes a gate electrode unit 105 (TG), a floating diffusion FD, and a contact CS, and these constitute the electronic reading unit that reads the fluorescence signal. This is as described in Example 1-1 above, and the description also applies to the present implementation.

In addition, as illustrated in the drawing, the pixel unit 181 may further include a gate electrode unit TG2, a floating diffusion FD2, and a contact CS2. These constitute an electron reading unit that reads the excitation light signal. This, too, is as described in Example 1-1 above, and the description also applies to the present implementation. Note that, in the drawing, the TG2 is not connected to the N regions 183, but the TG2 may be configured to be connected to the N regions 183. In this case, FD2 is unnecessary, and CS2 may be connected to the TG2.

### 1.1.8 Example 1-8 (Transparent Well)

In the present disclosure, the side wall portions of the well may be transparent. Specifically, the side wall portions may be transparent enough to transmit the excitation light. This configuration will be described with reference to FIG. 10.
(d) on a left side of the drawing is a schematic diagram of a cross section of a pixel unit 191 of an imaging element 190. The cross section is a cross section in a plane perpendicular to a light receiving surface of the imaging element.
(a) to (c) on a right side of the drawing are schematic diagrams illustrating a part of a light receiving surface of the imaging element 190 in which pixel units 191 are arranged in a lattice pattern, and more specifically, are schematic diagrams of cross sections as follows.
(a) on the right side of the drawing is a schematic diagram of a cross section at a position of broken line A-A′ in (d) of the drawing, and this schematic diagram illustrates a state in which the plurality of pixel units is arranged in a lattice pattern. In addition, the cross section is a cross section in a plane parallel to the light receiving surface of the imaging element.
(b) on the right side of the drawing is a schematic diagram of a cross section at a position of broken line B-B′ in (d) of the drawing, and this schematic diagram illustrates a state in which the plurality of pixel units is arranged in a lattice pattern. In addition, the cross section is a cross section in a plane parallel to the light receiving surface of the imaging element.
(c) on the right side of the drawing is a schematic diagram of a cross section at a position of broken line C-C′ in (d) of the drawing, and this schematic diagram illustrates a state in which the plurality of pixel units is arranged in a lattice pattern. In addition, the cross section is a cross section in a plane parallel to the light receiving surface of the imaging element.

The pixel unit 191 has a well 199 including a transparent material. An upper surface of the well 199 may be covered with a sheet 192 including a non-transparent material, as illustrated in the drawing.

A shape and size of the well 199 may be as described in Example 1-1 above, and the description also applies to the present example. In addition, the well 199 may be configured to hold an analyte as described in Example 1-1 above.

The pixel unit 191 includes a photodiode 194. The photodiode may be, for example, a Si photodiode, and may have, for example, an N region 194N and a P region 195P of Si. As illustrated in the drawing, the N region 194N may be surrounded by the P region 195P.

The pixel unit 191 includes a photodiode 194. The photodiode 194 may be provided only on a bottom side of the well.

In addition, the pixel unit 101 includes polysilicon (Poly-Si) 195 (TG). The polysilicon functions as a gate electrode unit. Furthermore, the pixel unit 101 includes a floating diffusion FD and a contact CS. These are as described in Example 1-1 above.

The pixel unit 191 is separated from other unit pixels by partitions 196. The partitions 196 may also be referred to as trenches.

Each partition 196 is provided between a certain unit pixel and another unit pixel. The partitions 196 may include an insulator or a metal. The partitions 196 are provided in such a way as to cover the photodiode 194, but need not be extended to the well 199. As a result, the excitation light L1 can travel in parallel with a light receiving surface. In addition, since the excitation light L1 travels in parallel with the light receiving surface, it is possible to prevent the excitation light from entering the photodiode 194. This contributes to improvement of the fluorescence detection accuracy.

The imaging element 190 is of a back-illuminated type, that is, a wiring layer is provided on a side of the photodiode opposite a fluorescence incident side.

A well 193 is provided on one side of the photodiode 194, and the wiring layer (not illustrated) is provided on an opposite side of the photodiode 194. That is, the pixel unit 191 has a multilayer structure in which the wiring layer, the detection unit (photodiode), and the analyte holding unit (well) are arranged in this order. With such a configuration, the imaging element in the present disclosure can obtain a larger fluorescence signal, which contributes to improvement of the fluorescence detection accuracy.

In a case where the imaging element 190 is used to analyze a biological sample, a space may be formed in order to cause an analyte to reach and be held in the well. The space may be a flow channel through which an analyte flows. For example, a liquid sample containing an analyte may flow through the space, and the analyte may be captured at the bottom surface of the well. At least a part of the space may be formed in a well shape. Another part of the space may be formed by a transparent substrate 198 as illustrated in FIG. 10. Since the transparent substrate 198 is transparent, the excitation light can reach an inside of the well. A material of the transparent substrate 198 may be, for example, glass, but may be a resin (for example, acrylic resin, polycarbonate resin, etc.).

The imaging element 190 may thus have a space for allowing an analyte to reach the inside of the well, and may further include the transparent substrate 198 that forms the space.

### 1.1.9 Example 1-9 (Transparent Well and Trenches)

The entirety of well side walls of the pixel unit described in Example 8 above is transparent. In the present disclosure, portions of the well side walls through which the excitation light travels may be transparent, and other portions may be formed by a photodiode. This configuration will be described with reference to FIG. 11.
(d) on a left side of the drawing is a schematic diagram of a cross section of a pixel unit 201 of an imaging element 200. The cross section is a cross section in a plane perpendicular to a light receiving surface of the imaging element.
(a) to (c) on a right side of the drawing are schematic diagrams illustrating a part of a light receiving surface of the imaging element 200 in which pixel units 201 are arranged in a lattice pattern, and more specifically, are schematic diagrams of cross sections as follows.
(a) on the right side of the drawing is a schematic diagram of a cross section at a position of broken line A-A′ in (d) of the drawing, and this schematic diagram illustrates a state in which the plurality of pixel units is arranged in a lattice pattern. In addition, the cross section is a cross section in a plane parallel to the light receiving surface of the imaging element.
(b) on the right side of the drawing is a schematic diagram of a cross section at a position of broken line B-B′ in (d) of the drawing, and this schematic diagram illustrates a state in which the plurality of pixel units is arranged in a lattice pattern. In addition, the cross section is a cross section in a plane parallel to the light receiving surface of the imaging element.
(c) on the right side of the drawing is a schematic diagram of a cross section at a position of broken line C-C′ in (d) of the drawing, and this schematic diagram illustrates a state in which the plurality of pixel units is arranged in a lattice pattern. In addition, the cross section is a cross section in a plane parallel to the light receiving surface of the imaging element.
(e) on the left side of the drawing is a schematic diagram of cross sections taken along broken line D-D′, broken line E-E′, and broken line F-F′ in (a), (b), and (c) of the drawing. The cross section is a cross section in a plane perpendicular to a light receiving surface of the imaging element.

The pixel unit 201 has a well in which an analyte is held as illustrated in (d) and (e) of the drawing.

As illustrated in (e) of the drawing, portions of the side walls of the well through which the excitation light L1 passes (that is, side walls orthogonal to the traveling direction of the excitation light L1) are transparent, so that the traveling of the excitation light is not hindered. The transparent side walls are not provided with a photodiode, and are not provided with partitions.

As illustrated in (d) of the drawing, on the other hand, among the side walls of the well, side walls parallel to the traveling direction of the excitation light L1 are formed by a photodiode, and partitions are provided.

Portions of the well included in the pixel unit of the imaging element in the present disclosure may thus be transparent, and other portions need not be transparent. The non-transparent portions may be formed by, for example, the photodiode. As a result, the excitation light can travel in parallel with the light receiving surface, and more fluorescence generated by the excitation light radiation can be received. This contributes to improvement of the fluorescence detection accuracy.

### 1.2 Configuration Example Relating to Excitation Light Blocking Unit

### 1.2.1 Example 2-1 (Basic Configuration Example)

In some examples of 1.1 described above, the configuration in which the multilayer film reflection filter is provided on the surface of the well has been described. In these examples, the multilayer film reflection filter is used as a component for preventing the excitation light from traveling to the detection unit (photodiode). That is, the multilayer film reflection filter is used as an excitation light blocking unit, and the excitation light blocking unit is provided on the analyte holding unit (well). That is, each pixel unit may be provided with an excitation light blocking unit that prevents the excitation light from reaching the detection unit. In addition, the excitation light blocking unit may be configured to transmit the fluorescence.

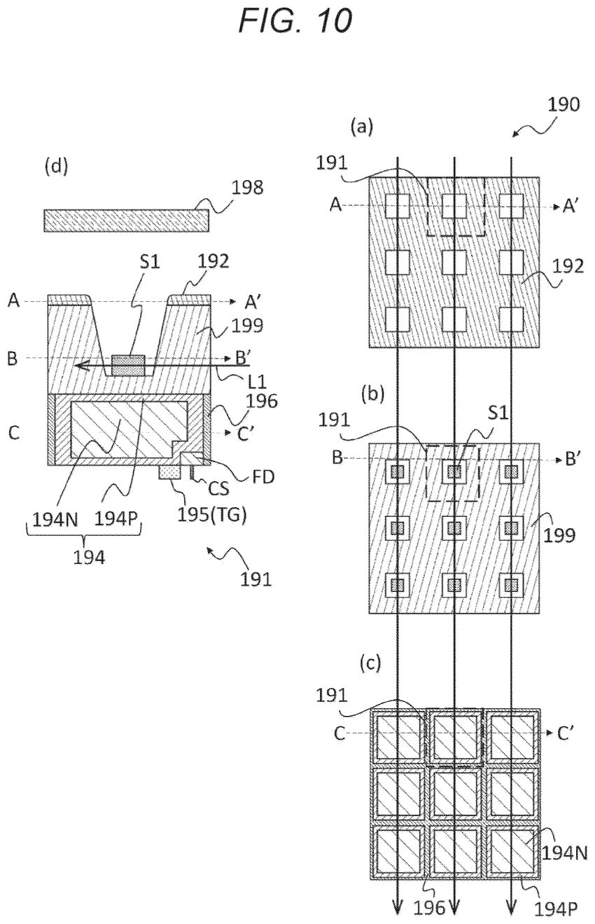

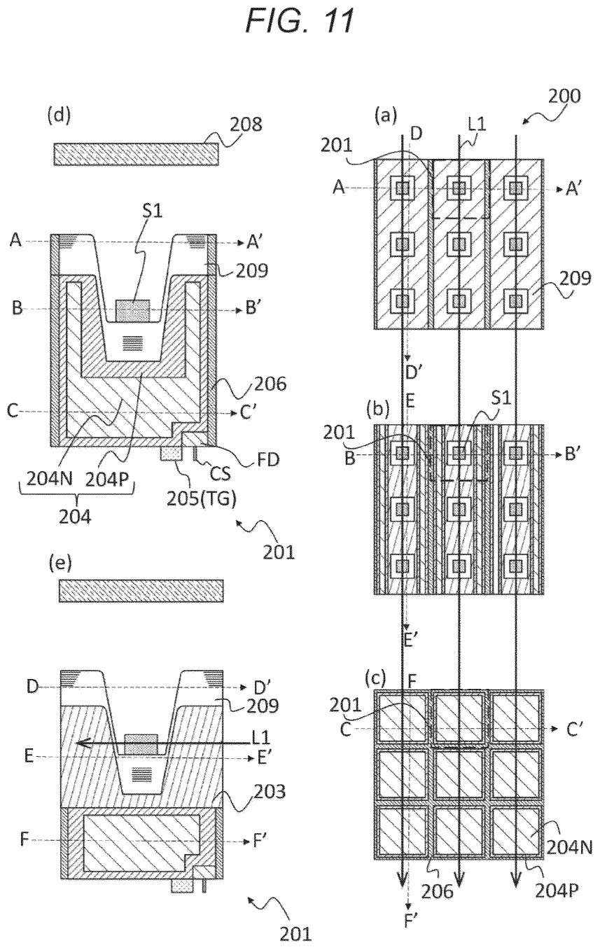

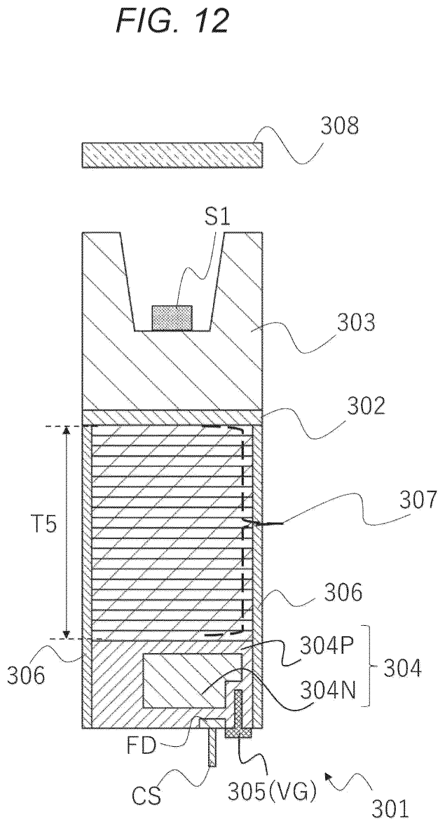

In the present disclosure, the excitation light blocking unit may be provided between the analyte holding unit (well) and the detection unit (photodiode). A configuration example of the back-illuminated imaging element according to the present disclosure including the excitation light blocking unit provided in this manner will be described hereinafter with reference to FIG. 12. The drawing is a schematic diagram of a cross section of the pixel unit of the back-illuminated imaging element according to the present disclosure. The cross section is a cross section in a plane perpendicular to a light receiving surface of the imaging element.

The pixel unit 301 includes a well 303 and a photodiode 304. The pixel unit 301 includes an insulating film 302 and a multilayer film reflection filter 307 between the well 303 and the photodiode 304. The multilayer film reflection filter 307 corresponds to the excitation light blocking unit described above. The pixel unit 301 may thus have a multilayer structure in which the photodiode 304, the multilayer film reflection filter 307, the insulating film 302, and the well 303 are arranged in this order.

The imaging element according to the present disclosure may include a plurality of pixel units 301 arranged in a lattice pattern as described in Example 1-1 above.

The well 303 may be configured to hold an analyte, that is, corresponds to the analyte holding unit.

A shape of the well 303 on a plane parallel to the light receiving surface may be a rectangular as described in Example 1-1 above, but may be another polygonal shape, or may be circular, elliptical, or the like, instead.

As described in Example 1-1 above, the well 303 may be configured to hold an analyte, that is, corresponds to the analyte holding unit.

Size of the well 303 and the pixel unit 301 may be as described for the well 103 and the pixel unit 101 in Example 1-1 above, and the description also applies to the present example. The well 303 may be configured to hold the analyte. For example, a compound for holding an analyte may be immobilized on a surface (particularly a bottom surface) of the well 303 as described in Example 1-1 above.

The photodiode 304 may be, for example, a Si photodiode, and may have, for example, an N region 304N and a P region 304P of Si. As illustrated in the drawing, the N region 304N may be surrounded by the P region 304P.

As illustrated in the drawing, the photodiode 304 may be provided only on a bottom side of the well.

The pixel unit 301 includes a gate electrode unit 305. The gate electrode unit 305 may be configured as a vertical transfer gate (VG). Alternatively, the gate electrode unit 305 may be configured as a transfer gate (TG). The gate electrode unit may include, for example, polysilicon (Poly-Si). Signal charges accumulated in the photodiode 304 are read via the gate electrode unit. The pixel unit 301 further includes a floating diffusion FD to which electrons accumulated in the photodiode are transferred, and a contact CS connected to the floating diffusion.

As illustrated in the drawing, the photodiode 304 may have an embedded photodiode structure. The gate electrode unit 305 is connected to the photodiode 304 having the structure, and electrons accumulated in the photodiode 304 are transferred from the gate electrode unit 305 to the floating diffusion FD, and then read from the contact CS.

In the present disclosure, a component provided in order to read electrons from the photodiode will also be referred to as an electronic reading unit. As described above, the electronic reading unit may include the gate electrode unit 305 (VG), the floating diffusion FD, and the contact CS. FD and VG may be in contact with each other or not be in contact with each other, and presence or absence of the contact can vary depending on an ON voltage of the gate electrode unit. In the drawing, the floating diffusion is provided in each pixel unit, that is, the electronic reading unit has a structure of a so-called FD non-sharing type, but the electronic reading unit may have a structure of the FD non-sharing type as described in Example 1-1 above.

The insulating film 302 may be, for example, a silicon oxide film, a nitrogen-containing silicon oxide film, a silicon nitride film, an oxygen-containing silicon nitride film, or a metal oxide film. The insulating film may be an insulating film subjected to high-density plasma processing.

The multilayer film reflection filter 307 may be as described above with reference to FIGS. 7A and 7B, and the description also applies to the present example. That is, the multilayer film reflection filter has a multilayer structure in which a layer H including a high refractive index material (hereinafter also referred to as a “high refractive index layer H”) and a layer L including a low refractive index material (hereinafter also referred to as a “low refractive index layer H”) are alternately stacked.

The multilayer film reflection filter may be configured such that the difference ΔT represented by the above Expression (I) is maximized.

In the present disclosure, the multilayer film reflection filter may thus be provided between the well (analyte holding unit) and the photodiode (detection unit). The multilayer film reflection filter blocks the excitation light radiated onto the well (and the analyte), and transmits the fluorescence generated from the analyte. As a result, noise caused by the excitation light can be reduced. This contributes to improvement of the fluorescence detection accuracy.

Thickness T5 of the multilayer film reflection filter 307 may be, for example, 100 nm to 30 μm, preferably 150 nm to 20 μm, and more preferably 200 nm to 15 μm.

The pixel unit 301 is separated from other unit pixels by partitions 306. The partitions 306 may also be referred to as trenches.

Each partition 306 is provided between a certain unit pixel and another unit pixel. The partitions 306 may include an insulator or a metal. The partitions 306 prevent the excitation light that has entered the unit pixel 301 and the fluorescence generated from the analyte S1 in the pixel unit 301 from entering other unit pixels. In addition, the partitions 306 prevent electrons in the photodiode 304 from entering photodiodes of other unit pixels.

The imaging element including the plurality of pixel units 301 in the present disclosure is of a back-illuminated type, that is, a wiring layer is provided on a side of the photodiode opposite a fluorescence incident side.

The well 303 is provided on one side of the photodiode 304, and the wiring layer is provided on an opposite side of the photodiode 304. That is, the pixel unit 201 has a multilayer structure in which the wiring layer, the detection unit (photodiode), and the analyte holding unit (well) are arranged in this order. With such a configuration, the imaging element in the present disclosure can obtain a larger fluorescence signal, which contributes to improvement of the fluorescence detection accuracy.

The imaging element including the plurality of pixel units 301 in the present disclosure may have a configuration in which the plurality of pixel units 301 is arranged in a lattice pattern as described in 1.1 above. The number of pixel units 301 included in one imaging element and the size and shape of the imaging element are as described in 1.1 above, and the description is also applied to the present example.

In a case where the imaging element including the plurality of pixel units 301 in the present disclosure is used to analyze a biological sample, a space may be formed in order to cause an analyte to reach and be held in the well. The space may be a flow channel through which an analyte flows. For example, a liquid sample containing an analyte may flow through the space, and the analyte may be captured at the bottom surface of the well. At least a part of the space may be formed in a well shape. Another part of the space may be formed by a transparent substrate 308 as illustrated in FIG. 12. Since the transparent substrate 308 is transparent, the excitation light can reach an inside of the well. A material of the transparent substrate 308 may be, for example, glass, but may be a resin (for example, acrylic resin, polycarbonate resin, etc.).

The imaging element in the present disclosure may thus have a space for allowing an analyte to reach the inside of the well, and may further include the transparent substrate 308 that forms the space.

### 1.2.2 Example 2-2 (Photodiode with Two-Layer Structure)

A photodiode for detecting excitation light may be further added to the pixel unit described in Example 1-1 above. That is, in the present disclosure, each pixel unit may further include an excitation light detection unit that detects the excitation light. The back-illuminated imaging element in the present disclosure may be configured to process a signal obtained by the fluorescence detection unit using a signal obtained by the excitation light detection unit.

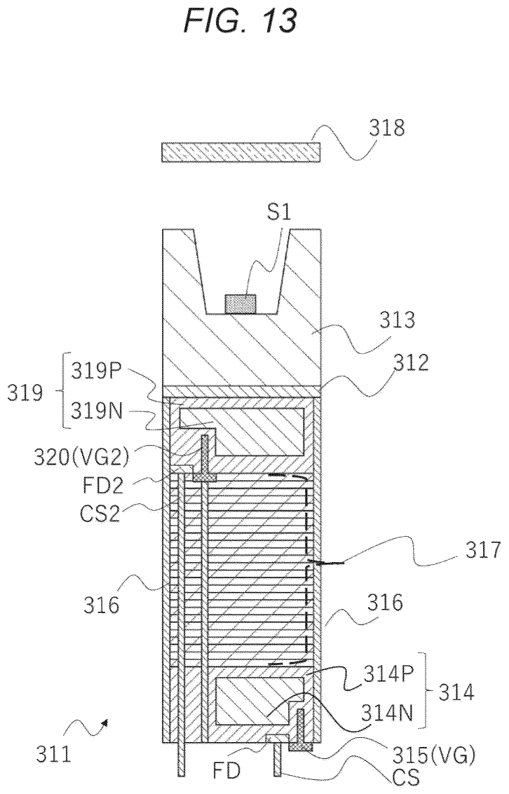

A configuration example of the pixel unit to which the photodiode is added will be described hereinafter with reference to FIG. 13. The drawing is a schematic diagram of a cross section of the pixel unit of the back-illuminated imaging element according to the present disclosure. The cross section is a cross section in a plane perpendicular to a light receiving surface of the imaging element.

The pixel unit 311 includes a well 313 and a fluorescence detection photodiode 314. The pixel unit 311 includes an insulating film 312, a photodiode 319 for detecting excitation light, and a multilayer film reflection filter 317 between the well 313 and the photodiode 314. The multilayer film reflection filter 317 corresponds to the excitation light blocking unit described above. The pixel unit 311 may thus have a multilayer structure in which the fluorescence detection photodiode 314, the multilayer film reflection filter 317, the excitation light detection photodiode 319, the insulating film 312, and the well 313 are arranged in this order.

The imaging element according to the present disclosure may include a plurality of pixel units 311 arranged in a lattice pattern as described in Example 1-1 above.

The fluorescence detection photodiode 314, the gate electrode unit 315 (VG), the floating diffusion FD, the contact CS, the multilayer film reflection filter 317, the insulating film 312, and the well 313 may be the same as the photodiode 304 for detecting fluorescence, the multilayer film reflection filter 307, the insulating film 302, and the well 303 described in Example 2-1, and the description in 2-1 also applies to the present example.

The excitation light detection photodiode 319 may be, for example, a Si photodiode, and may have, for example, an N region 319N and a P region 319P of Si. As illustrated in the drawing, the N region 319N may be surrounded by the P region 319P.

As illustrated in the drawing, the excitation light detection photodiode 319 may be provided between the well 313 and the multilayer film reflection filter 317.

The excitation light detection photodiode 319 may be provided with a gate electrode unit 320. Signal charges accumulated in the excitation light photodiode 319 are read via the gate electrode unit.

The gate electrode unit 320 (VG2) is configured to read electrons of the excitation light detection photodiode 319. The gate electrode unit 320 (VG2) has a portion that reads electrons and a portion that passes through the multilayer film reflection filter 317 and the fluorescence detection photodiode 314. The former portion may include, for example, polysilicon (Poly-Si). The latter portion may be, for example, a metal electrode covered with an insulating film, and the metal electrode can be connected to the wiring layer.

In addition, the pixel unit 311 may further include a floating diffusion FD to which electrons accumulated in the excitation light detection photodiode are transferred, and a contact CS connected to the floating diffusion.

As illustrated in the drawing, the photodiode 319 may have an embedded photodiode structure. The gate electrode unit 320 (VG2) is connected to the photodiode 319 having the structure, and electrons accumulated in the photodiode 319 are transferred from the gate electrode unit 320 to the floating diffusion FD, and then read from the contact CS.

The excitation light detection photodiode can detect the signal of the excitation light. On the basis of the excitation light signal, excitation light noise that can be included in the fluorescence signal can be reduced or removed. This contributes to improvement of the fluorescence detection accuracy.

### 1.2.3 Example 2-3 (Photodiode with Two-Layer Structure and Absorption Filter)

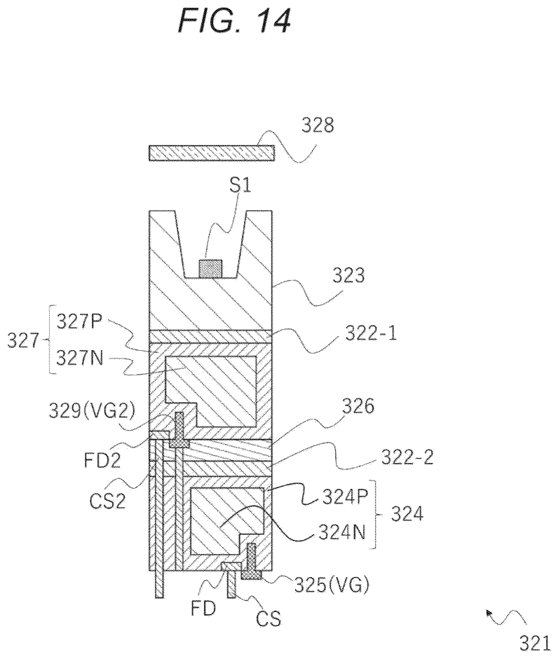

In the pixel unit described in Example 1-1 above, a multilayer film reflection filter is provided between the two photodiodes. In the present disclosure, an absorption filter having optical characteristics of absorbing excitation light may be provided between the two photodiodes instead of the multilayer film reflection filter. The absorption filter has optical characteristics of transmitting fluorescence. A configuration example of a pixel unit including the absorption filter will be described hereinafter with reference to FIG. 14. The drawing is a schematic diagram of a cross section of the pixel unit of the back-illuminated imaging element according to the present disclosure. The cross section is a cross section in a plane perpendicular to a light receiving surface of the imaging element.

The pixel unit 321 includes a well 323 and a fluorescence detection photodiode 324. The pixel unit 321 includes an insulating film 322-1, an excitation light detection photodiode 327, an absorption filter 326, and an insulating film 322-2 between the well 323 and the photodiode 324. The absorption filter 326 corresponds to the excitation light blocking unit described above. The pixel unit 321 may thus have a multilayer structure in which the fluorescence detection photodiode 324, the absorption filter 326, the excitation light detection photodiode 327, and the well 323 are arranged in this order.

The imaging element according to the present disclosure may include a plurality of pixel units 321 arranged in a lattice pattern as described in Example 1-1 above.

The fluorescence detection photodiode 324, a gate electrode unit 325 (VG), a floating diffusion FD, a contact CS, the insulating film 322-1, the insulating film 322-2, and the well 323 may be the same as the fluorescence detection photodiode 304, the gate electrode unit 305, the floating diffusion FD, the contact CS, the insulating film 302, and the well 303 described in Example 2-1 above, and the description in Example 2-1 also applies to the present example.

The excitation light detection photodiode 327, a gate electrode unit 329, a floating diffusion FD2, and a contact CS2 may be the same as the excitation light detection photodiode 319, the gate electrode unit 320, the floating diffusion FD2, and the contact CS2 described in Example 2-2 above, and the description in Example 2-2 also applies to the present example.

The absorption filter 326 may be a filter having optical characteristics for selectively transmitting light in a specific wavelength range and absorbing light in other wavelength ranges. The absorption filter 326 may be a filter having optical characteristics for selectively transmitting fluorescence and absorbing at least excitation light.

In the present disclosure, such an absorption filter is provided between the excitation light detection photodiode and the fluorescence detection photodiode. The absorption filter can prevent the excitation light from traveling to the fluorescence detection photodiode, which contributes to improvement of the fluorescence detection accuracy. In addition, since the absorption filter is disposed in front of the excitation light detection photodiode on an optical path of the excitation light, the absorption filter does not adversely affect excitation light detection accuracy of the excitation light detection photodiode.

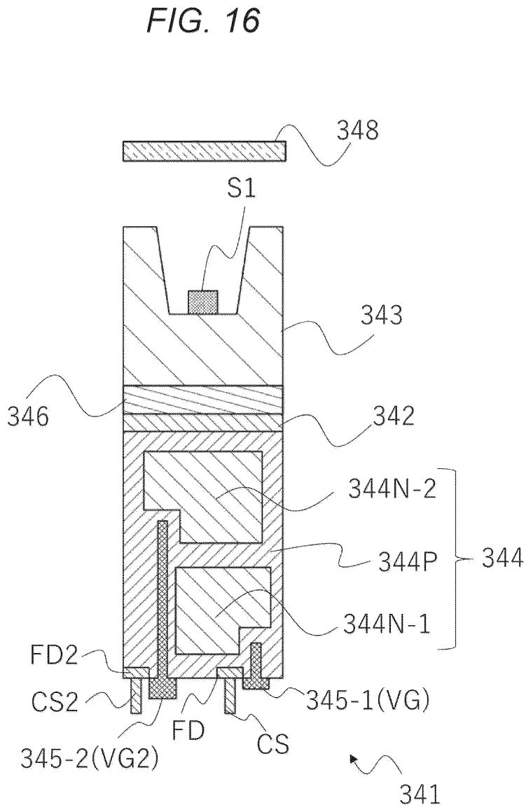

In addition, in the present disclosure, as illustrated in FIG. 16, an N region 344N-1 for detecting fluorescence and an N region 344N-2 for detecting excitation light may be provided in one P region 344P. The P region 344P and the N region 344N-1 function as the fluorescence detection photodiode. Electrons accumulated in the fluorescence detection photodiode are read by an electronic reading unit (a gate electrode unit 345-1, the floating diffusion FD, and the contact CS).

The P region 344P and the N region 344N-2 function as the excitation light detection photodiode. Electrons accumulated in the excitation light detection photodiode are read by an electronic reading unit (a gate electrode unit 345-2, the floating diffusion FD2, and the contact CS2).

In the present disclosure, each pixel unit may be configured to include a photodiode having such a two-layer structure. This configuration will be described later in more detail in Example 4-1 and subsequent examples.

### 1.2.4 Example 2-4 (Use of Photoelectric Conversion Film)

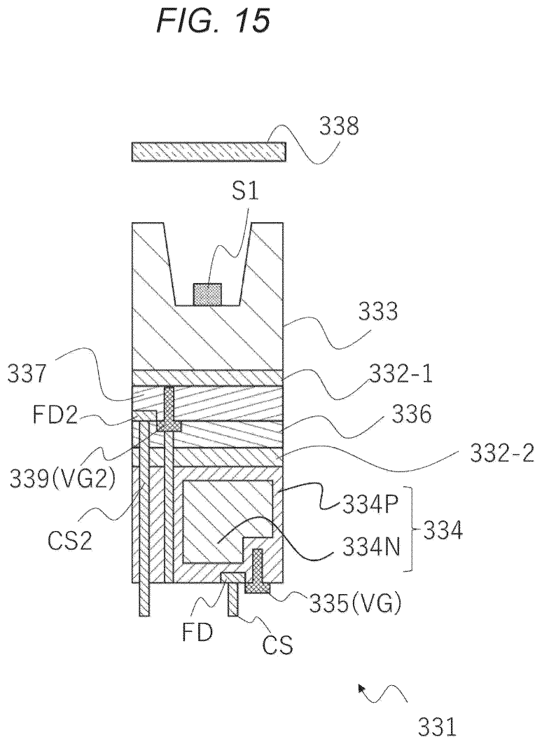

The pixel unit described in Example 2-3 above includes an excitation light detection photodiode. In the present disclosure, a photoelectric conversion film may be provided instead of the excitation light detection photodiode. The photoelectric conversion film may be a photoelectric conversion film having wavelength selectivity, and in particular, may be a photoelectric conversion film that selectively performs photoelectric conversion on excitation light. The excitation light may thus be detected by the photoelectric conversion film. A configuration example of a pixel unit including the photoelectric conversion film will be described hereinafter with reference to FIG. 15. The drawing is a schematic diagram of a cross section of the pixel unit of the back-illuminated imaging element according to the present disclosure. The cross section is a cross section in a plane perpendicular to a light receiving surface of the imaging element.

The pixel unit 331 includes a well 333 and a fluorescence detection photodiode 334. The pixel unit 331 includes an insulating film 332-1, a photoelectric conversion film 337, an absorption filter 336, and an insulating film 322-2 between the well 333 and the photodiode 334. The absorption filter 336 corresponds to the excitation light blocking unit described above. The pixel unit 331 may thus have a multilayer structure in which the fluorescence detection photodiode 334, the absorption filter 336, the photoelectric conversion film 337, and the well 333 are arranged in this order.

The imaging element according to the present disclosure may include a plurality of pixel units 331 arranged in a lattice pattern as described in Example 1-1 above.

The fluorescence detection photodiode 334, a gate electrode unit 335, a floating diffusion FD, a contact CS, the insulating film 332-1, the insulating film 332-2, and the well 333 may be the same as the fluorescence detection photodiode 304, the gate electrode unit 305, the floating diffusion FD, the contact CS, the insulating film 302, and the well 303 described in Example 2-1 above, and the description in Example 2-1 also applies to the present example.

The absorption filter 336 may be the same as the absorption filter 326 described in Example 2-3 above, and the description in Example 2-3 also applies to the present example.

The photoelectric conversion film 337 may be a photoelectric conversion film having wavelength selectivity, and in particular, may be a photoelectric conversion film that selectively performs photoelectric conversion on excitation light radiated onto an analyte. The photoelectric conversion film 337 need not perform photoelectric conversion on fluorescence generated by irradiating the analyte with the excitation light. The photoelectric conversion film may include, for example, an inorganic material or an organic material.

The inorganic material may be, for example, an inorganic semiconductor material. In one implementation, the inorganic semiconductor material may be a group III-V semiconductor material, and may be, for example, a gallium arsenide-based semiconductor or an indium phosphide-based semiconductor. More specific examples of such a material include InGaAs, GaAs, InP, and GaN, and in particular, the material may be InGaAs. In another implementation, the inorganic material may be a two-dimensional semiconductor material, and the material may be MoS₂ or WS₂. In still another implementation, the inorganic material may be GaO₃.

The organic material may be, for example, an organic semiconductor material. In a case where the photoelectric conversion film includes the organic semiconductor material, the photoelectric conversion film may have one or more layers selected from, for example, a p-type organic semiconductor layer, an n-type organic semiconductor layer, and a mixed layer of a p-type organic semiconductor and an n-type organic semiconductor.

For example, the photoelectric conversion film may have a single-layer structure of a p-type organic semiconductor, a single-layer structure of an n-type organic semiconductor, or a single-layer structure of a mixed layer of a p-type organic semiconductor and an n-type organic semiconductor.

In addition, the photoelectric conversion film may have a multilayer structure of a p-type organic semiconductor layer and an n-type organic semiconductor layer, that is, a multilayer structure of “a p-type organic semiconductor layer/an n-type organic semiconductor layer”. In addition, the photoelectric conversion film may have a structure in which a p-type organic semiconductor layer and/or an n-type organic semiconductor layer and the mixed layer are stacked on each other. For example, the photoelectric conversion film may have a multilayer structure of “a p-type organic semiconductor layer/a mixed layer of a p-type organic semiconductor and an n-type organic semiconductor/an n-type organic semiconductor layer”, a multilayer structure of “a p-type organic semiconductor layer/a mixed layer of a p-type organic semiconductor and an n-type organic semiconductor”, or a multilayer structure of “an n-type organic semiconductor layer/a mixed layer of a p-type organic semiconductor and an n-type organic semiconductor”.

Examples of the p-type organic semiconductor include a naphthalene derivative, an anthracene derivative, a phenanthrene derivative, a pyrene derivative, a perylene derivative, a tetracene derivative, a pentacene derivative, a quinacridone derivative, a thiophene derivative, a thienothiophene derivative, a benzothiophene derivative, a benzothienobenzothiophene derivative, a triallylamine derivative, a carbazole derivative, a perylene derivative, a picene derivative, a chrysene derivative, a fluoranthene derivative, a phthalocyanine derivative, a subphthalocyanine derivative, a subporphyrazine derivative, a metal complex having a heterocyclic compound as a ligand, a polythiophene derivative, a polybenzothiadiazole derivative, a polyfluorene derivative, and the like. The p-type organic semiconductor may be one, or two or more, of these.

Examples of the n-type organic semiconductor include fullerenes and fullerene derivatives <e.g., fullerenes (higher fullerenes, endohedral fullerenes, etc.) such as C60, C70, and C74 or fullerene derivatives (e.g., fullerene fluorides, PCBM fullerene compounds, fullerene polymers, etc.)>, organic semiconductors with higher (deeper) HOMO and LUMO levels than p-type organic semiconductors, and transparent inorganic metal oxides. The n-type organic semiconductor may be one, or two or more, of these.

More specifically, examples of the n-type organic semiconductor include heterocyclic compounds containing nitrogen, oxygen, or sulfur atoms, such as organic molecules, organometallic complexes, and subphthalocyanine derivatives that incorporate a pyridine derivative, a pyrazine derivative, a pyrimidine derivative, a triazine derivative, a quinoline derivative, a quinoxaline derivative, an isoquinoline derivative, an acridine derivative, a phenazine derivative, a phenanthroline derivative, a tetrazole derivative, a pyrazole derivative, an imidazole derivative, a thiazole derivative, an oxazole derivative, an imidazole derivative, a benzimidazole derivative, a benzotriazole derivative, a benzoxazole derivative, a benzoxazole derivatives, a carbazole derivative, a benzofuran derivatives, a dibenzofuran derivative, a subporphyrazine derivative, a polyphenylenevinylene derivative, a polybenzothiadiazole derivative, or a polyfluorene derivatives as part of a molecular backbone thereof.

Examples of groups and the like contained in the fullerene derivatives include halogen atoms; linear, branched, or cyclic alkyl groups or phenyl groups; groups containing linear or fused aromatic compounds; groups containing halides; partial fluoroalkyl groups; perfluoroalkyl groups; silylalkyl groups; silylalkoxy groups; arylsilyl groups; arylsulfanyl groups; alkylsulfanyl groups; arylsulfonyl groups; alkylsulfonyl groups; arylsulfide groups; alkylsulfide groups; amino groups; alkylamino groups; arylamino groups; hydroxy groups; alkoxy groups; acylamino groups; acyloxy groups; carbonyl groups; carboxyl groups; carboxamide groups; carboalkoxy groups; acyl groups; sulfonyl groups; cyano groups; nitro groups; groups containing chalcogen compounds; phosphine groups; phosphonyl groups; and derivatives of these.

Film thickness of the photoelectric conversion film may be, for example, 1×10⁻⁸ m (meters) to 5×10⁻⁷ m, preferably 2.5×10⁻⁸ m to 3× 10⁻⁷ m, more preferably 2.5×10⁻⁸ m to 2×10⁻⁷ m, and still more preferably 1×10⁻⁷ m to 1.8×10⁻⁷ m. Note that although the organic semiconductor is often classified into a p-type and an n-type, the p-type means that holes are easily transported and the n-type means that electrons are easily transported, and the organic semiconductor is not limited to an interpretation that it has holes or electrons as a majority carrier of thermal excitation like the inorganic semiconductor.

Examples of a material constituting a photoelectric conversion film that performs photoelectric conversion on light having a green wavelength include rhodamine-based dyes, melacyanine-based dyes, quinacridone derivatives, subphthalocyanine-based dyes (subphthalocyanine derivatives), and the like.

In addition, examples of a material constituting a photoelectric conversion film for performing photoelectric conversion on blue light include a coumaric acid dye, tris-8-hydroxyquinoline aluminum (Alq3), a melacyanine dye, and the like.

Furthermore, examples of a material constituting a photoelectric conversion film for performing photoelectic conversion on red light include phthalocyanine-based dyes and subphthalocyanine-based dyes (subphthalocyanine derivatives).

Furthermore, as the photoelectric conversion film, a panchromatic photosensitive organic photoelectric conversion film that is sensitive to substantially all visible light from an ultraviolet region to a red region can be used.

Signal charges generated by the photoelectric conversion by the photoelectric conversion film 337 may be read by, for example, a gate electrode unit 339 provided in the photoelectric conversion film 337. Furthermore, the photoelectric conversion film 337 may be provided with a floating diffusion FD2 and a contact CS2. The gate electrode unit 339, the floating diffusion FD2, and the contact CS2 may be the same as the gate electrode unit 320, the floating diffusion FD2, and the contact CS2 described in Example 2-2 above, and the description in Example 2-2 also applies to the present example.

Noise reduction processing of a fluorescence signal detected by the fluorescence detection photodiode 334 can be performed on the basis of the read signal charges. The fluorescence detection accuracy can thus be improved.

### 1.2.5 Example 2-5 (Another Configuration Example of Photodiode with Two-Layer Structure and Absorption Filter)

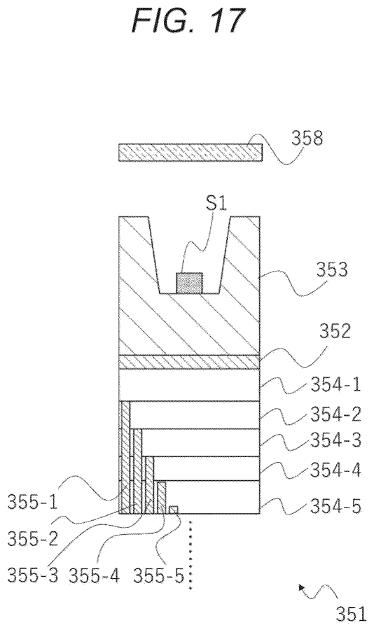

The pixel units described in Examples 2-1 to 2-4 above include a fluorescence detection photodiode. In the present disclosure, a quantum dot photoelectric conversion film may be used instead of the fluorescence detection photodiode, and for example, a multilayer film in which a plurality of quantum dot photoelectric conversion films is stacked on each other may be used. An example of the pixel unit configured in this manner will be described hereinafter with reference to FIG. 17. The drawing is a schematic diagram of a cross section of the pixel unit of the back-illuminated imaging element according to the present disclosure. The cross section is a cross section in a plane perpendicular to a light receiving surface of the imaging element.

A pixel unit 351 includes a multilayer film in which quantum dot photoelectric conversion films 354-1 to 354-5 are stacked on each other in addition to the well 353. An insulating film 352 is provided between the well 353 and the multilayer film.

Each of the quantum dot photoelectric conversion films performs photoelectric conversion on light having a specific wavelength. That is, the multilayer film includes a plurality of quantum dot photoelectric conversion films that performs photoelectric conversion on light (in particular, fluorescence) having wavelengths different from each other. This makes it possible to detect fluorescence of various wavelengths.

In addition, one or more of the plurality of quantum dot photoelectric conversion films may perform photoelectric conversion on excitation light. The excitation light may thus be detected.

Each quantum dot photoelectric conversion film may be connected to a corresponding one of electronic reading units 355-1 to 355-5. A signal in each quantum dot photoelectric conversion film connected to the corresponding electronic reading unit is read from each electronic reading unit. As described above, each electronic reading unit may have a gate electrode unit (TG or VG). In addition, each electronic reading unit may include a floating diffusion FD and/or a contact CS.

Note that the pixel unit in the drawing includes a multilayer film in which five quantum dot photoelectric conversion films are stacked on each other, but the number of quantum dot photoelectric conversion films included in the pixel unit is not limited to five. In the present disclosure, the pixel unit may include one quantum dot photoelectric conversion film, or may include two or more quantum dot photoelectric conversion films.

A wavelength of light to be subjected to photoelectric conversion varies depending on particle size of quantum dots. By changing the particle size of the quantum dots, therefore, it is possible to cope with various types of fluorescence.

### 1.2.6 Example 2-6 (Stacking of Absorption Filter on Transparent Substrate)

As described above, the imaging element in the present disclosure is used in combination with a transparent substrate to form a flow channel. That is, the present disclosure also provides a flow channel unit including the imaging element and the transparent substrate, in which the imaging element and the transparent substrate form a flow channel.

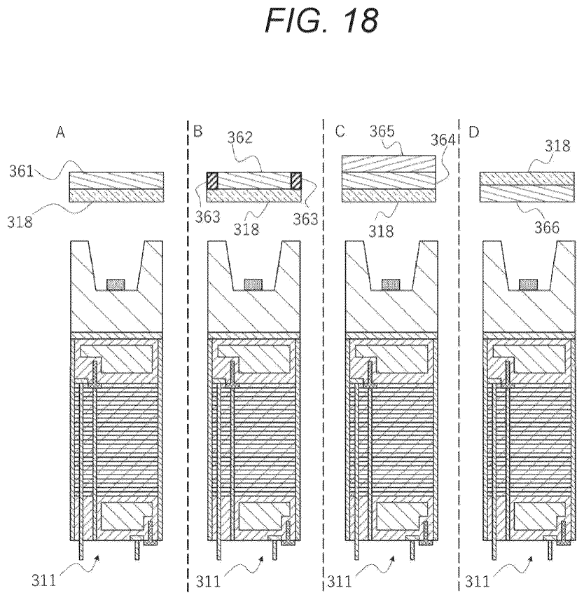

In the present disclosure, an absorption filter may be stacked on the transparent substrate. A configuration example relating to a transparent substrate on which an absorption filter is stacked will be described with reference to FIG. 18.

In any of A to D of the drawing, a pixel unit 311 is illustrated. The pixel unit 311 is as described in Example 2-2 above with reference to FIG. 13, and the description also applies to the present example.

In one implementation, as illustrated in A of the drawing, an absorption filter 361 may be stacked on a surface opposite a surface on a well side among two main surfaces of the transparent substrate 318.

In another implementation, as illustrated in B of the drawing, an absorption filter 362 may be stacked on the surface opposite the surface on the well side among the two main surfaces of the transparent substrate 318, and the absorption filter 362 may be separated for each pixel by partitions 363.

In still another implementation, as illustrated in C of the drawing, two absorption filters 364 and 365 may be stacked on the surface opposite the surface on the well side among the two main surfaces of the transparent substrate 318. The two absorption filters 364 and 365 may be configured to absorb light having different wavelengths.

Note that the number of absorption filters stacked on one main surface of a transparent substrate is not limited to one or two, and may be three or more, instead.

In still another implementation, as illustrated in D of the drawing, an absorption filter 366 may be stacked on the surface on the well side among the two main surfaces of the transparent substrate 318.

Note that although one absorption filter is stacked on the surface in D of the drawing, two or more absorption filters may be stacked on the surface, and the two or more absorption filters may be configured to absorb light having different wavelengths.

As described above, by stacking the absorption filter on the transparent substrate, unnecessary light can be absorbed, and only necessary excitation light can reach the analyte. In addition, it is also possible to prevent unnecessary light from reaching the fluorescence detection photodiode. This contributes to improvement of the fluorescence detection accuracy.

Furthermore, by using the absorption filter, it is possible to construct a biological sample analysis system without using an expensive and narrowband light source such as an LED or a laser

### 1.2.7 Example 2-7 (Modifications of Well)

In the present disclosure, an uneven shape may be provided on the bottom surface of the well (that is, the surface on which the analyte is held).

In addition, in the present disclosure, partitions may be provided around the well in such a way as to define the pixel unit.

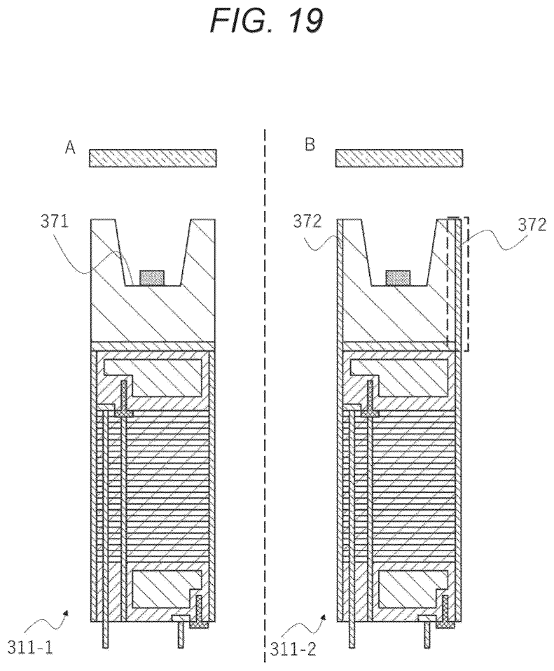

These configuration examples will be described hereinafter with reference to FIG. 19.

A pixel unit 311-1 of an imaging element illustrated in A of the drawing is the same as the pixel unit 311 of the imaging element described in Example 2-2 above with reference to FIG. 13, except that an uneven shape is provided on a well bottom surface 371 of the well 313.

In the present disclosure, as in the pixel unit 311-1 of the imaging element, an uneven shape may be provided on the surface of the bottom of the well. For example, a convex structure having, for example, a conical shape, a cylindrical shape, a cubic shape, a rectangular parallelepiped shape, or a pyramid shape (for example, a triangular pyramid shape, a quadrangular pyramid shape, or a pentagonal pyramid shape) may be regularly or irregularly provided on the surface. Alternatively, a concave structure having, for example, a conical shape, a cylindrical shape, a cubic shape, a rectangular parallelepiped shape, or a pyramid shape (for example, a triangular pyramid shape, a quadrangular pyramid shape, or a pentagonal pyramid shape) may be regularly or irregularly provided on the surface.

Such a surface can prevent surface reflection of fluorescence at the bottom surface of the well. Such a surface can therefore generate more fluorescence, which contributes to improvement of the fluorescence detection accuracy.

Dimensions of the convex or concave structure may preferably be smaller than a wavelength of fluorescence to be detected. A structure having such small dimensions is particularly suitable for preventing reflection of fluorescence.

A pixel unit 311-2 of an imaging element illustrated in B of the drawing is the same as the pixel unit 311 of the imaging element described in Example 2-2 above with reference to FIG. 13, except that partitions 372 are provided in such a way as to surround the well 313. That is, the pixel unit 311-2 includes a partition 372 in a portion surrounded by a broken line, but the pixel unit 311 does not have a partition in that portion. The partitions 372 separate the pixel unit also in the well. The partitions 372 may include an insulator or a metal.

In the present disclosure, since the partitions are provided around the well, it is possible to prevent fluorescence generated from an analyte in the well or excitation light radiated onto the well from traveling to photodiodes of other pixel units. As a result, noise caused by light in other pixel units can be reduced, which contributes to improvement of the fluorescence detection accuracy.

### 1.2.8 Example 2-8 (Inclination of Well)

In the present disclosure, the well may be provided such that the bottom surface of the well is inclined with respect to a stacking surface between the fluorescence detection photodiode and the multilayer film reflection filter. In order to provide the well in an inclined manner, for example, an insulation film supporting the well may be inclined.

In addition, in a case where the well is provided in an inclined manner, a lens may be provided in the insulating film. The lens may be a lens having optical characteristics of condensing at least part of the fluorescence at the fluorescence detection photodiode.

Note that the lens may have optical characteristics of condensing at least part of the excitation light at the excitation light detection photodiode.

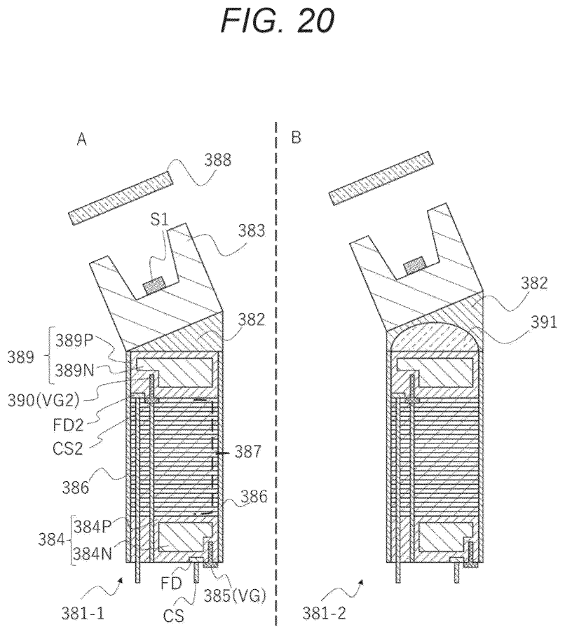

A configuration example in which the well is inclined will be described hereinafter with reference to FIG. 20.

A pixel unit 381-1 of an imaging element illustrated in A of the drawing includes a fluorescence detection photodiode 384, a gate electrode unit 385 connected to the fluorescence detection photodiode, a floating diffusion FD, a contact CS, partitions 386, a multilayer film reflection filter 387, an excitation light detection photodiode 389, a gate electrode unit 390 connected to the excitation light detection photodiode, a floating diffusion FD2, and a contact CS2. These are the same as the fluorescence detection photodiode 314, the gate electrode unit 315, the floating diffusion FD, the contact CS, the partitions 316, the multilayer film reflection filter 317, the excitation light detection photodiode 319, the gate electrode unit 320, the floating diffusion FD2, and the contact CS2 described in Example 2-2 above with reference to FIG. 13, and the description also applies to the present example. In addition, a transparent substrate 388 is also the same as the transparent substrate 318 described in Example 2-2 above with reference to FIG. 13.

The pixel unit 381-1 includes a well 383. The well is provided in such a way as to be inclined with respect to an incident surface (that is, a stacking surface between the fluorescence detection photodiode 384 and the multilayer film reflection filter 387) through which the fluorescence enters the fluorescence detection photodiode 384. That is, the bottom surface of the well holding the analyte is inclined with respect to the incident surface (that is, the stacking surface).

In order to incline the well as described above, the pixel unit 381-1 is configured such that an insulating film 382 provided between the well 383 and the excitation light detection photodiode 389 is inclined. That is, two main surfaces of the insulating film are provided in such a way as not to be parallel to each other but to cross each other (that is, in such a way as to form an angle of more than 0 degrees).

Alternatively, an upper surface (a surface on which the excitation light is incident) of the excitation light photodiode may be inclined, and in this case, the two main surfaces of the insulating film may be parallel to each other.

In a case where light traveling straight (for example, laser light) is used as the excitation light, since the well (in particular, the bottom surface of the well on which the analyte is held) is inclined in this manner, the excitation light incident on the fluorescence detection photodiode can be reduced. This results in reduction of noise due to excitation light and contributes to improvement of the fluorescence detection accuracy.

An imaging element 381-2 illustrated in B of the drawing is the same as the pixel unit 381-1 of the imaging element illustrated in A of the drawing, except that a lens 391 is provided inside the insulating film 382.

The lens 391 may be configured to condense at least part of the fluorescence generated by irradiating the analyte S1 with the excitation light at the fluorescence detection photodiode 384.

Note that a shape of the lens 391 is not limited to a convex lens illustrated in the drawing, and may be a diffraction lens or the like, instead.

By providing the lens, more fluorescence can be incident on the photodiode. This contributes to improvement of the fluorescence detection accuracy.

### 1.2.9 Example 2-9 (Shifting of Well)

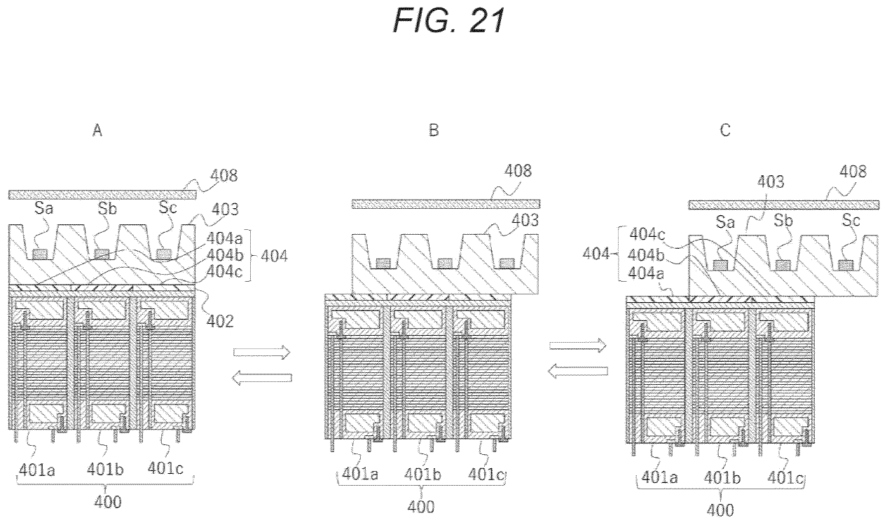

The imaging element in the present disclosure may be configured such that a position of the well can be shifted with respect to a position of the fluorescence detection photodiode. An example of an imaging element configured in this manner will be described hereinafter with reference to FIG. 21.

A of the drawing illustrates an imaging element 400 in which a plurality of pixel units 401a, 401b, and 401c is arranged. These pixel units are the same as the pixel unit 311 described in the insulating film Example 2-2 above with reference to FIG. 13 except that a position of a well 403 is movable with respect to the position of the fluorescence detecting photodiode and that a color filter 404 is provided. The color filter 404 includes color filter regions 404a, 404b, and 404c. The color filter region 404a provided in the pixel unit 401a, the color filter region 404b provided in the pixel unit 402b, and the color filter region 404c provided in the pixel unit 401c transmit light of different wavelengths. Note that the term “color filter region” may be simply referred to as a “color filter” by omitting the “region” from the viewpoint of attention to each pixel unit.

A of the drawing illustrates a state before the well 403 is moved. B of the drawing illustrates a state in the middle of the movement of the well 403, and C of the drawing illustrates a state after the movement of the well 403. As illustrated in these diagrams, the well 403 can move with respect to the position of the fluorescence detection photodiode. The excitation light detection photodiode and the multilayer film reflection filter, on the other hand, are fixed in such a way as not to be movable with respect to the position of the fluorescence detection photodiode. In addition, the transparent substrate 408 may also be movable in accordance with the movement.

The three types of pixel units 401a, 401b, and 401c illustrated in these diagrams are respectively provided with color filters 404a, 404b, and 404c that transmit light of different wavelengths. As with the excitation light detection photodiode and the multilayer film reflection filter, these color filters may be fixed in such a way as not to be movable with respect to the position of the fluorescence detection photodiode.

In addition, positions where the color filters are provided may be appropriately selected. For example, the color filters may be provided on an insulating film 402 as illustrated in the drawing, may be provided between the insulating film and the excitation light detection photodiode, may be provided between the excitation light detection photodiode and the multilayer film reflection filter, or may be provided between the multilayer film reflection filter and the fluorescence detection photodiode.

As illustrated in A of the drawing, before the well 403 is moved, an analyte Sb is held in a well of the pixel unit 401b at the center. As illustrated in C of the drawing, after the well 403 is moved, the well holding the analyte Sb and the analyte Sb moves onto the pixel unit 401c on a right side. Here, a wavelength of light transmitted by the color filter 404b existing on the pixel unit 401b is different from a wavelength of light transmitted by the color filter 404c existing on the right pixel unit 401c. As a result, light generated from the analyte Sb need not be detected by, for example, the central pixel unit 401b, but may be detected by the right pixel unit 401c after the movement. Conversely, the light generated from the analyte may be detected by, for example, the central pixel unit 401b, but need not be detected by the right pixel unit 401c after the movement. As a result, it is possible to distinguish a wavelength band of the light generated by the analyte. In addition, light of two wavelengths can be observed in one sequence of light detection in a state before the movement and light detection in a state after the movement. This improves analysis speed.

In the mode described above, one well moves over two pixels as a result of the movement. In this case, the color filters may be configured to transmit light of two different wavelengths depending on the position.

In addition, the color filters may be configured to transmit light of three or more different wavelengths depending on the position of the well, and light of three or more different wavelengths may be detected in one sequence by controlling the position of the well.

Since the well can be shifted in this manner, an effect of being capable of detecting light generated from an analyte with a plurality of pixels is exhibited.

Note that, in the drawing referred to above, the three color filters 404a, 404b, and 404c have optical characteristics of transmitting light of different wavelengths, but the configuration of the color filters is not limited thereto. For example, the color filter 404a and the color filter 404c may transmit light of the same wavelength, and the wavelength of light transmitted by the color filter 404b may be different from that of light transmitted by the color filter 404a and the color filter 404c. In addition, the color filter 404a and the color filter 404b may transmit light of the same wavelength, and the wavelength of light transmitted by the color filter 404c may be different from that of light transmitted by the color filter 404a and the color filter 404b.

In addition, the drawing referred to above illustrates the three types of color filter regions 404a, 404b, and 404c that transmit light of different wavelengths, that is, the three types of color filters having different optical characteristics. In the present disclosure, the number of types of optical characteristics of the color filter regions is not limited to three. For example, the color filters may have two or more types of color filter regions that transmit light having wavelengths different from each other, and may have, for example, two to ten, particularly two, three, or four types of color filter regions.

### 1.2.10 Example 2-10 (Use of Waveguide, Absorption Filter, Plasmonic Filter, Metamaterial, or Polarizer)

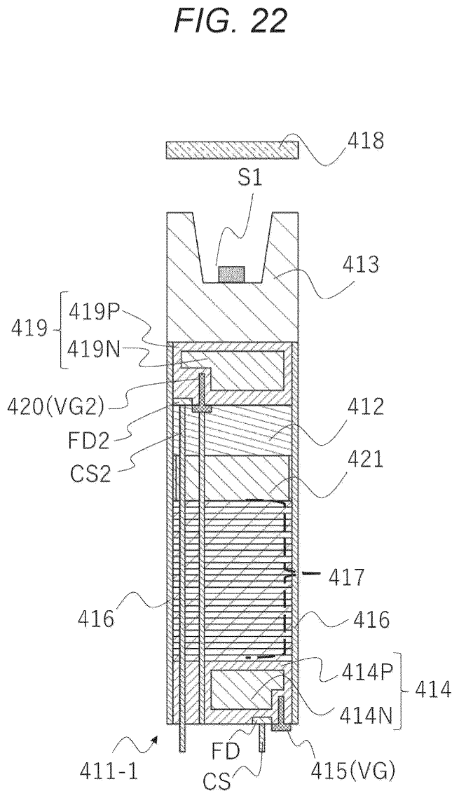

The imaging element in the present disclosure may include one or more of a waveguide, an absorption filter, a plasmonic filter, a metamaterial, and a polarizer between the fluorescence detection photodiode and the well. Examples of imaging elements configured in this manner will be described hereinafter with reference to FIGS. 22 to 29.

A pixel unit 411-1 illustrated in FIG. 22 includes a well 413, a fluorescence detection photodiode 414, a gate electrode unit 415 connected to the fluorescence detection photodiode, a floating diffusion FD, a contact CS, partitions 416, a multilayer film reflection filter 417, an excitation light detection photodiode 419, a gate electrode unit 420 connected to the excitation light detection photodiode, a floating diffusion FD2, and a contact CS2. These are the same as the well 313, the fluorescence detection photodiode 314, the gate electrode unit 315, the floating diffusion FD, the contact CS, the partitions 316, the multilayer film reflection filter 317, the excitation light detection photodiode 319, the gate electrode unit 320, the floating diffusion FD2, and the contact CS2 described in Example 2-2 above with reference to FIG. 13, and the description also applies to the present example. In addition, a transparent substrate 418 is also the same as the transparent substrate 318 described in Example 2-2 above with reference to FIG. 13.

The pixel unit 411-1 includes an absorption filter 412 and a waveguide 421 between the fluorescence detection photodiode 414 and the well 413. More specifically, the pixel unit 411-1 has a multilayer structure in which the fluorescence detection photodiode 414, the multilayer film reflection filter 417, the waveguide 421, the absorption filter 412, the excitation light detection photodiode 419, and the well 413 are arranged in this order.

For example, the absorption filter 412 may have optical characteristics of absorbing excitation light. Furthermore, the absorption filter 412 has optical characteristics of transmitting fluorescence. The absorption filter 412 makes it possible to prevent unnecessary light other than fluorescence from reaching the fluorescence detection photodiode 414. This contributes to improvement of the fluorescence detection accuracy.

In addition, since the waveguide 421 is present between the absorption filter 412 and the multilayer film reflection filter 417, fluorescence can more reliably reach the fluorescence detection photodiode. This contributes to improvement of the fluorescence detection accuracy.

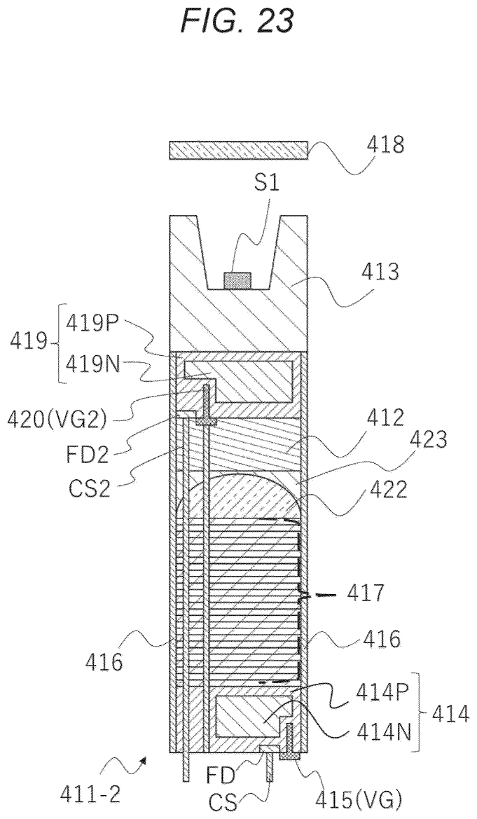

A pixel unit 411-2 illustrated in FIG. 23 includes a waveguide. Instead of the waveguide, a lens 422 or a diffraction grating may be provided as illustrated in the drawing. The lens or the diffraction grating may be configured to collect fluorescence, and in particular, may be configured to collect fluorescence with the fluorescence detection photodiode. This improves the fluorescence detection accuracy.

In addition, the lens or the diffraction grating may have wavelength dependency. More specifically, the lens or the diffraction grating may be configured to condense light of a specific wavelength at a specific position and condense light of another specific wavelength at another specific position. That is, a light collection point can be changed in accordance with the wavelength.

In addition, as illustrated in the drawing, two or more N regions may be formed in one P region, that is, two or more photodiodes may be formed. Since the light collection point changes in accordance with the wavelength and two or more photodiodes are formed, light can be dispersed.

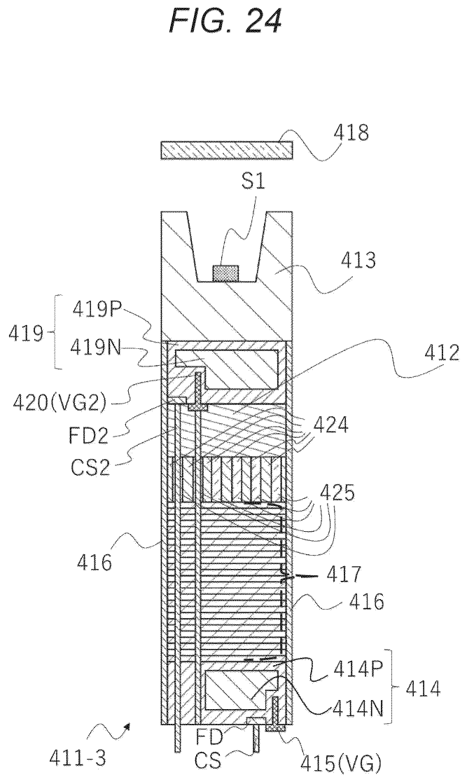

A pixel unit 411-3 illustrated in FIG. 24 includes waveguides 424 arranged in an array. The pixel unit 411-3 is the same as the pixel unit 411-1 illustrated in FIG. 22 referred to above, except that waveguides are arranged in an array. In addition, the waveguides may be formed in an insulating film 425.

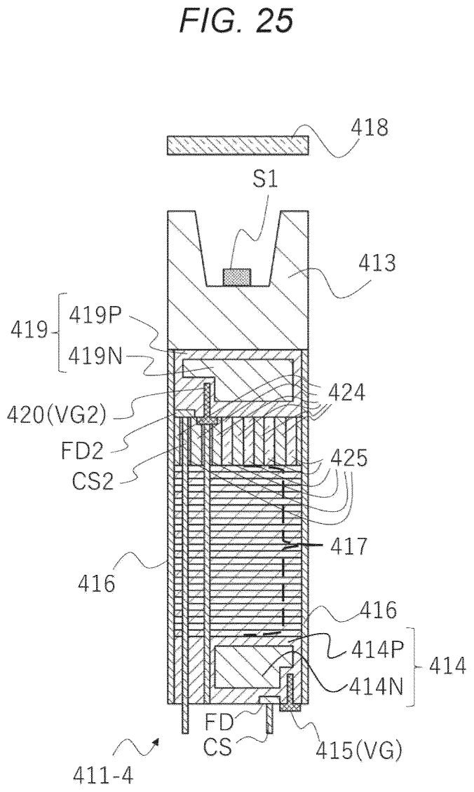

A pixel unit 411-4 illustrated in FIG. 25 is the same as the pixel unit 411-3 illustrated in FIG. 24 referred to above, except that the absorption filter 412 is not provided. The imaging element in the present disclosure thus need not include an absorption filter, and may include waveguides and a multilayer film reflection filter.

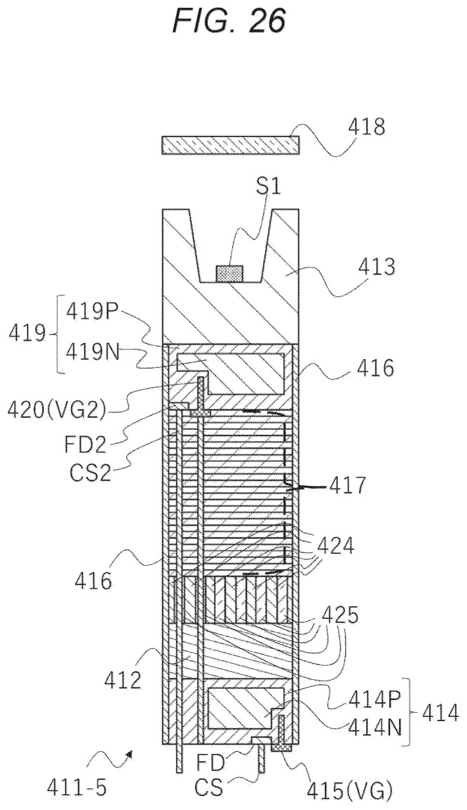

A pixel unit 411-5 illustrated in FIG. 26 is the same as the pixel unit 411-3 illustrated in FIG. 24 except that order of stacking of the absorption filter 412, the waveguides 424, and the multilayer film reflection filter 417 is different. That is, the pixel unit 411-5 has a multilayer structure in which the fluorescence detection photodiode 414, the absorption filter 412, the waveguide 424, the multilayer film reflection filter 417, the excitation light detection photodiode 419, and the well 413 are arranged in this order.

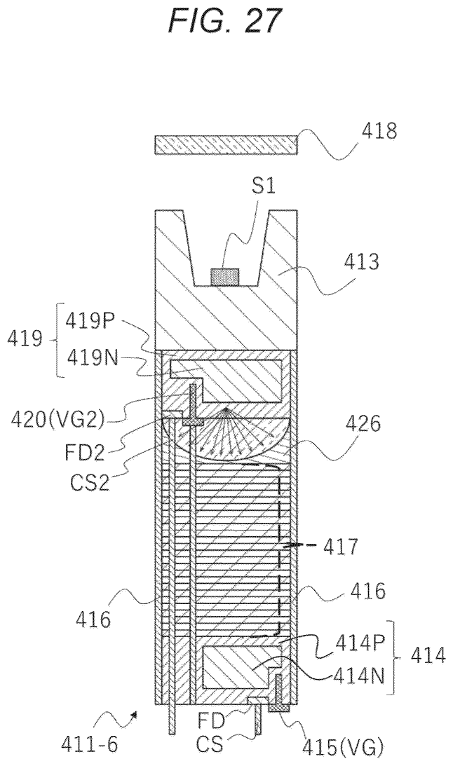

In the pixel unit 411-6 illustrated in FIG. 27, a lens structure is formed between the multilayer film reflection filter 417 and the excitation light detection photodiode. The lens structure may be, for example, a lens structure configured to function as a lens by adjusting a shape of an absorption filter 426. In the drawing, a concave portion is schematically formed in the absorption filter 426, and a structure of the concave portion is configured to function as a lens. Such a shape of the concave lens can prevent color mixing.

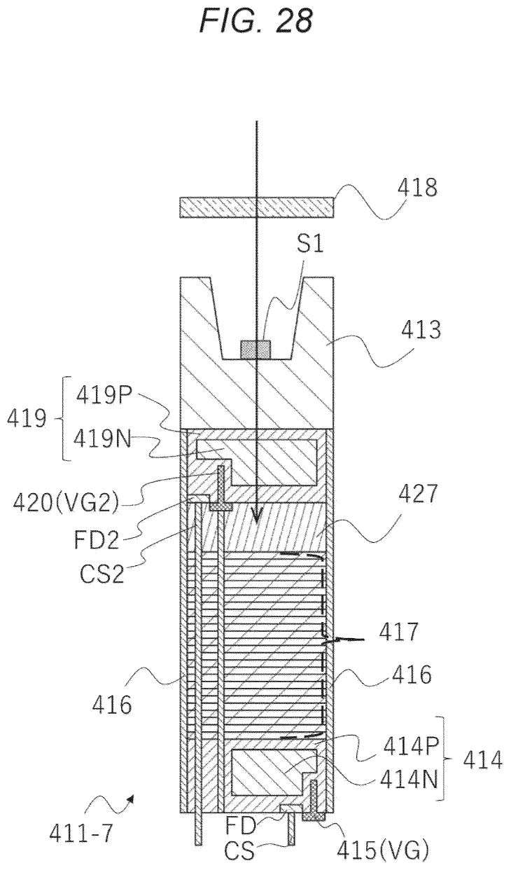

In a pixel unit 411-7 illustrated in FIG. 28, a plasmonic filter 427 is formed between the multilayer film reflection filter 417 and the excitation light detection photodiode. The plasmonic filter has optical characteristics of transmitting only light having a specific wavelength. The plasmonic filter, therefore, may be configured to transmit, for example, only fluorescence. The plasmonic filter may contain metal particles (particularly nanoparticles), and may contain, for example, gold (Au) particles.

Alternatively, the pixel unit may have a metamaterial instead of the plasmonic filter 427. More particularly, the pixel unit may include a layer including a metamaterial (also referred to as a “metamaterial layer”) between the multilayer film reflection filter 417 and the excitation light detection photodiode. The metamaterial layer may have optical characteristics of transmitting only light having a specific wavelength, and in particular, may be configured to selectively transmit fluorescence to be detected by the fluorescence detection photodiode.

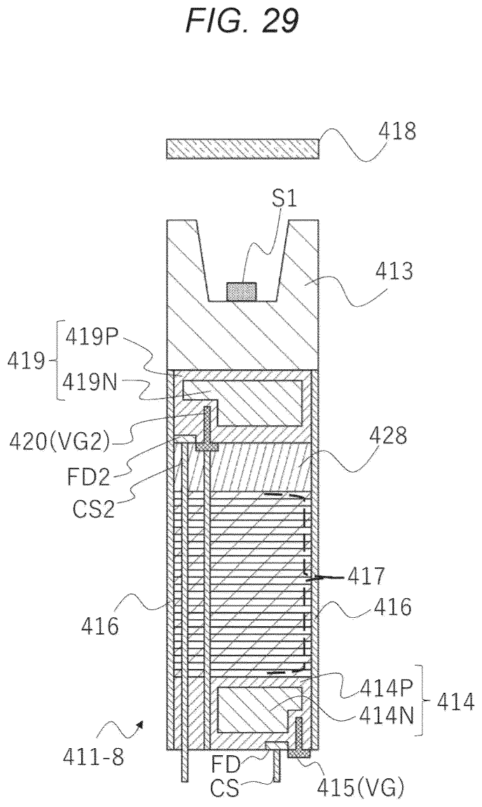

In a pixel unit 411-8 illustrated in FIG. 29, a polarizer 428 is formed between the multilayer film reflection filter 417 and the excitation light detection photodiode. In a case where the polarizer is used, the excitation light is preferably polarized light. The polarizer may have optical characteristics of quenching polarized light that is the excitation light and transmitting fluorescence. Note that a mode using a polarizer will be described in more detail later.

With the above configuration, too, the fluorescence detection accuracy can be improved.

### 1.2.11 Example 2-11 (Fluorescence Reflection Material Disposed Above Well)

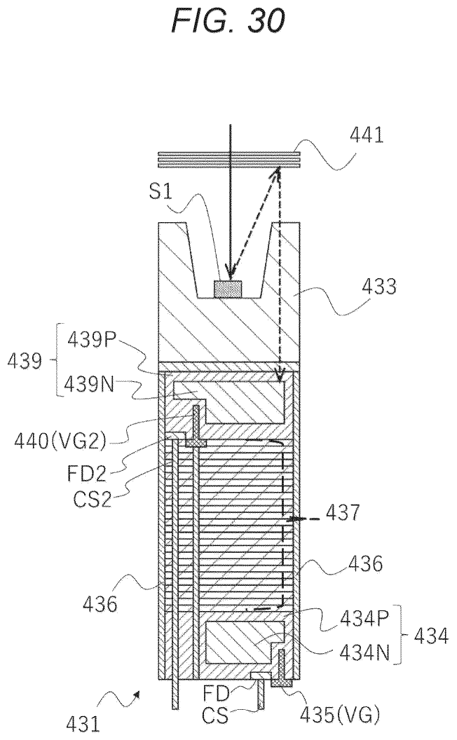

In the present disclosure, a material that reflects fluorescence generated by irradiating an analyte with excitation light may be disposed above the well. In particular, the material may transmit the excitation light. The material may be, for example, a multilayer film. This configuration will be described hereinafter with reference to FIG. 30.

A pixel unit 431 illustrated in the drawing includes a well 433, a fluorescence detection photodiode 434, a gate electrode unit 435 connected to the fluorescence detection photodiode, a floating diffusion FD, a contact CS, partitions 436, a multilayer film reflection filter 437, an excitation light detection photodiode 439, a gate electrode unit 440 connected to the excitation light detection photodiode, a floating diffusion FD2, and a contact CS2. These are the same as the well 313, the fluorescence detection photodiode 314, the gate electrode unit 315, the floating diffusion FD, the contact CS, the partitions 316, the multilayer film reflection filter 317, the excitation light detection photodiode 319, the gate electrode unit 320, the floating diffusion FD2, and the contact CS2 described in Example 2-2 above with reference to FIG. 13, and the description also applies to the present example.

As illustrated in the drawing, a multilayer film 441 is provided in such a way as to be disposed immediately above the well, particularly above the analyte. That is, the multilayer film is provided in such a way as to sandwich the analyte S1 between the multilayer film 441 and the well 433. The multilayer film may be stacked on, for example, the transparent substrate described above.

The stacked film reflects fluorescence (broken line arrows) generated by irradiating the analyte with excitation light and transmits the excitation light. By the radiation of the excitation light, fluorescence traveling toward the fluorescence detection photodiode is generated, but fluorescence traveling toward a side opposite the fluorescence detection photodiode (that is, a well opening side) can also be generated. The fluorescence traveling to the opposite side is reflected by the multilayer film and detected by the fluorescence detection photodiode. This contributes to improvement of the fluorescence detection accuracy.

### 1.2.12 Example 2-12 (Pinhole Structure)

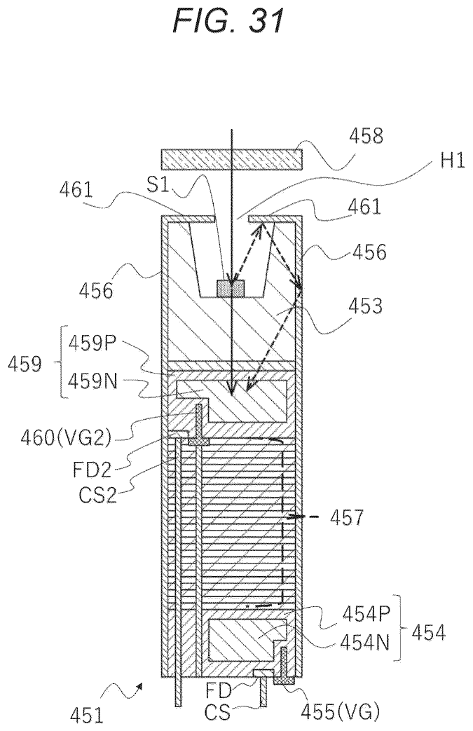

In the present disclosure, a pinhole structure or a MEMS shutter for reflecting fluorescence generated by irradiating an analyte with excitation light may be provided above the well. This configuration will be described hereinafter with reference to FIG. 31.

A pixel unit 451 illustrated in the drawing includes a well 453, a fluorescence detection photodiode 454, a gate electrode unit 455 connected to the fluorescence detection photodiode, a floating diffusion FD, a contact CS, a multilayer film reflection filter 457, an excitation light detection photodiode 459, a gate electrode unit 460 connected to the excitation light detection photodiode, a floating diffusion FD2, and a contact CS2. These are the same as the well 313, the fluorescence detection photodiode 314, the gate electrode unit 315, the floating diffusion FD, the contact CS, the multilayer film reflection filter 317, the excitation light detection photodiode 319, the gate electrode unit 320, the floating diffusion FD2, and the contact CS2 described in Example 2-2 above with reference to FIG. 13, and the description also applies to the present example.

As illustrated in the drawing, a lid 461 having a pinhole H1 is provided in an opening of the well 453.

Since the pinhole H1 is provided in the lid 461, it is possible to irradiate the analyte S1 with the excitation light (solid line arrows in the drawing).

An inner surface (a surface on a well side) of the lid 461 may include, for example, a material that reflects fluorescence (a metal such as Al). As a result, fluorescence (broken line arrows in the drawing) generated by irradiating the analyte S1 with the excitation light is reflected by the lid 461 as illustrated in the drawing without going out of the well, and travels toward the fluorescence detection photodiode 454.

In addition, as illustrated in the drawing, partitions 456 are extended in such a way as to reach the lid. For example, the partitions 456 may be configured as light guide walls that guide fluorescence to the fluorescence detection photodiode 454. For example, the partitions 456 may include a clad material or a metal (a light reflective material that is easy to embed, such as, for example, Cu, W, or Ti) having a low refractive index. In addition, in some implementations, the partitions 456 may be air gaps.

The lid and the partitions described above can increase the amount of fluorescence reaching the fluorescence detection photodiode, which contributes to improvement of the fluorescence detection accuracy.

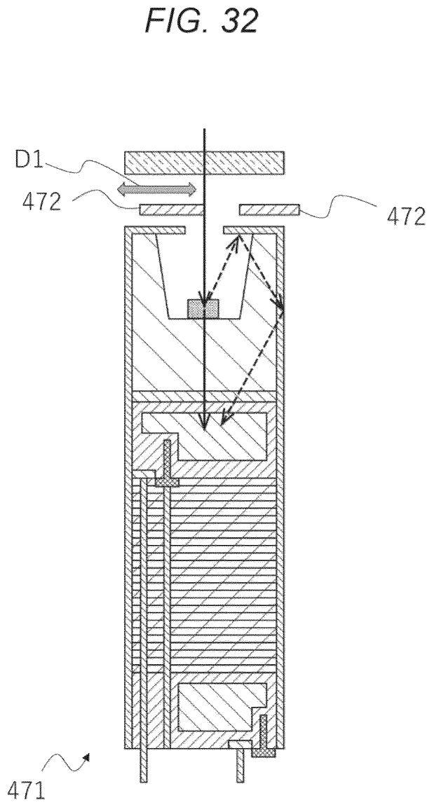

Alternatively, the lid 461 may be a MEMS shutter. An example of a case where a MEMS shutter is used will be described with reference to FIG. 32. As illustrated in the drawing, a pixel unit 371 of an imaging element has a configuration in which a MEMS shutter 372 is added to the lid of the pixel unit 361 of the imaging element illustrated in FIG. 31. Opening and closing of the pinhole is controlled by moving the MEMS shutter 372 in a direction of an arrow D1 in the drawing.

### 1.2.13 Example 2-13 (Excitation Light Blocking Unit Provided on Si One-Layer Structure)

The pixel units described in Example 2-11 above with reference to FIGS. 22 to 27 include a fluorescence detection photodiode and an excitation light detection photodiode, that is, has a Si two-layer structure, and further includes a multilayer film reflection filter and an excitation light blocking unit such as an absorption filter or a waveguide in addition to the Si two-layer structure.

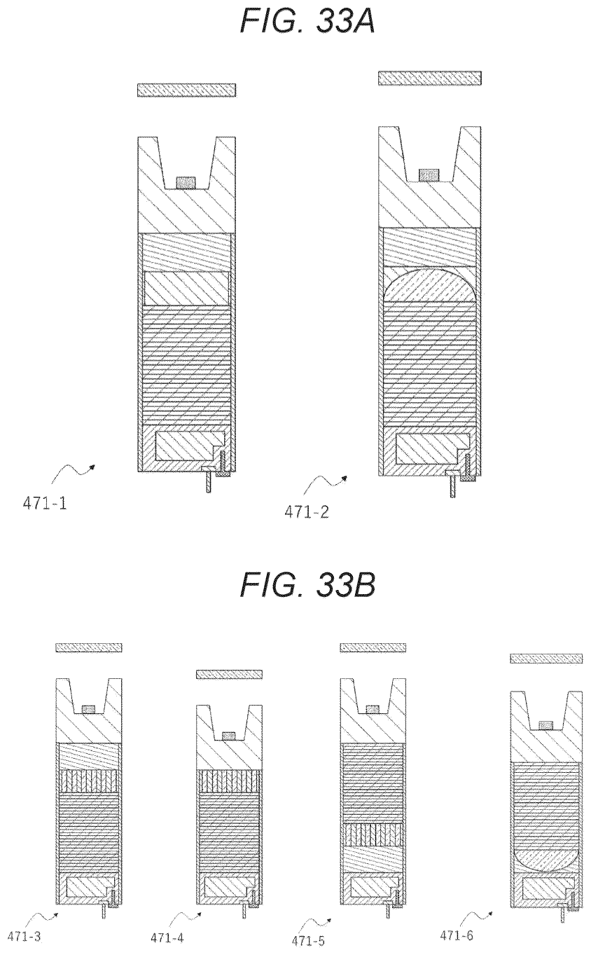

In the present disclosure, the pixel unit may be configured not to include the excitation light detection photodiode but to include the fluorescence detection photodiode and the excitation light blocking unit. This configuration will be described hereinafter with reference to FIGS. 33A and 33B.

Pixel units 471-1 to 471-6 illustrated in FIGS. 33A and 33B are similar to the pixel units 411-1 to 411-6 illustrated in FIGS. 22 to 27 except that the pixel units do not include an excitation light detection photodiode and a gate electrode unit connected to the excitation light detection photodiode.

The pixel unit in the present disclosure may thus have a stacked structure of the fluorescence detection photodiode and the excitation light blocking unit (a multilayer film reflection filter and an absorption filter, a waveguide, or a lens).

### 1.2.14 Example 2-14 (Excitation Light Blocking Unit Provided on Si Two-Layer Structure)

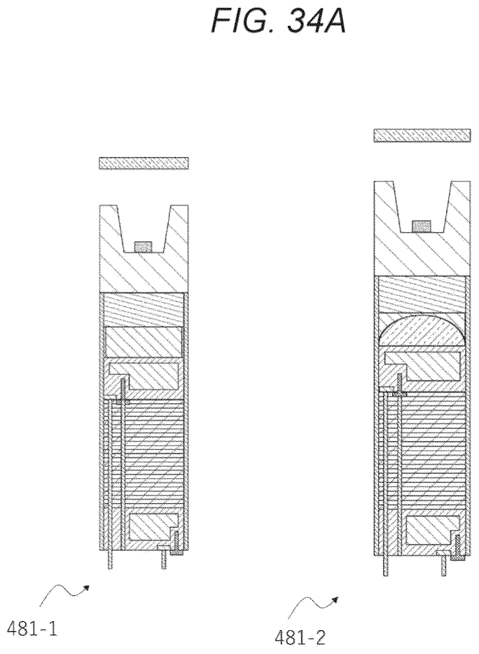

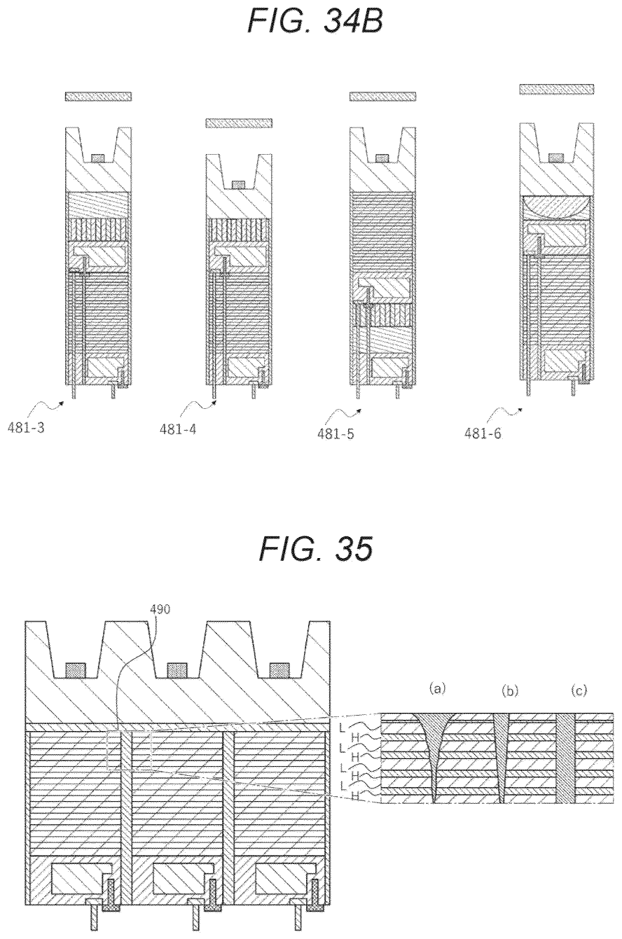

The pixel units described in Example 2-11 above with reference to FIGS. 22 to 27 include an excitation light blocking unit between the fluorescence detection photodiode and the excitation light detection photodiode. In the present disclosure, in the pixel unit, some of components of the excitation light blocking unit may be provided between the fluorescence detection photodiode and the excitation light detection photodiode, and other components of the excitation light blocking unit may be provided between the excitation light detection photodiode and the well. This configuration will be described hereinafter with reference to FIGS. 34A and 34B.

Pixel units 481-1 to 481-6 illustrated in FIGS. 34A and 34B are similar to the pixel units 411-1 to 411-6 illustrated in FIGS. 22 to 27 except that some of the components of the excitation light blocking unit are between the well and the excitation light detection photodiode, and the other components of the excitation light blocking unit are between the well and the excitation light detection photodiode. The pixel unit in the present disclosure may be configured as described above.

### 1.2.15 Example 2-15 (Trench Shape)

In the imaging element in which a plurality of pixel units is arranged described in Example 2-1 above, multilayer film reflection filters of two adjacent pixel units are separated from each other by a partition as illustrated in a left region 490 of FIG. 35. A trench shape of the partitions may be, for example, any one of shapes (a) to (c) on a right side of the drawing. These shapes will be described hereinafter.

As illustrated in (a) of the drawing, the trench shape may be a shape in which a substantially rectangular parallelepiped shape in the high refractive index layer H and a substantially trapezoidal shape in the low refractive index layer L are alternately stacked. That is, in the high refractive index layer H, the multilayer film reflection filter may be dug vertically, and in the low refractive index layer L, the multilayer film reflection filter may be dug in such a way as to have a taper angle.

Alternatively, the multilayer film reflection filter may be dug in such a way as to have a taper angle in both the high refractive index layer H and the boat refractive index layer L. As a result, the trench shape may be a shape in which width becomes narrower from a shallow portion toward a deep portion of the multilayer film reflection filter as illustrated in (b) of the drawing.

Alternatively, the multilayer film reflection filter may be dug in such a way as not to have a taper angle in both the high refractive index layer H and the boat refractive index layer L. As a result, the trench shape may be a shape having a constant width at any depth of the multilayer film reflection filter as illustrated in (c) of the drawing.

### 1.3 Configuration Example Relating to Excitation Light Blocking Unit Including Polarizer

### 1.3.1 Example 3-1 (Basic Configuration Example)

The back-illuminated imaging element according to the present disclosure may include an excitation light blocking unit as described in 1.2 above. In one implementation, the excitation light blocking unit may include a polarizer. In this case, the excitation light may be, for example, polarized light.

A configuration example of the back-illuminated imaging element in the present disclosure including a polarizer will be described hereinafter with reference to the drawings.

In a solid-state imaging device that detects a sample such as a biologically derived substance, a high excitation light cut ratio (high S/N ratio) and miniaturization of pixel size (high throughput) are required.

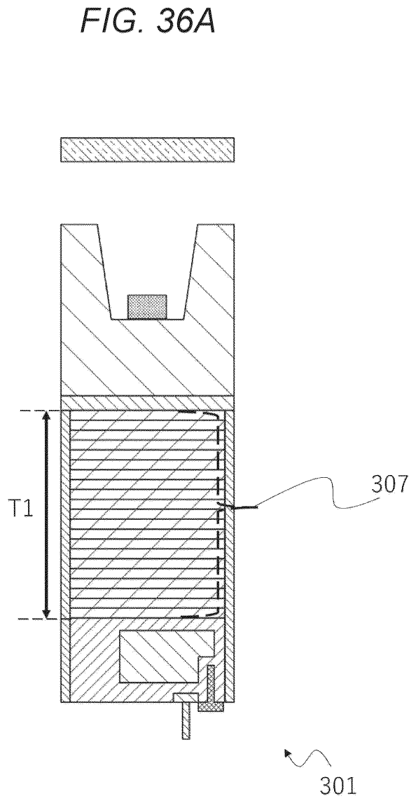

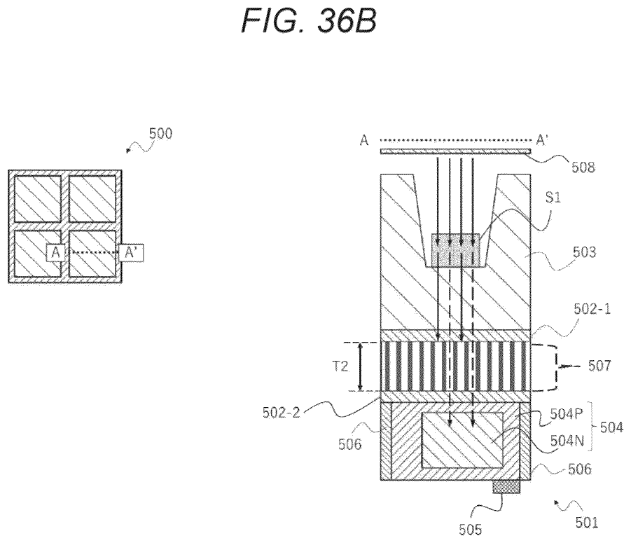

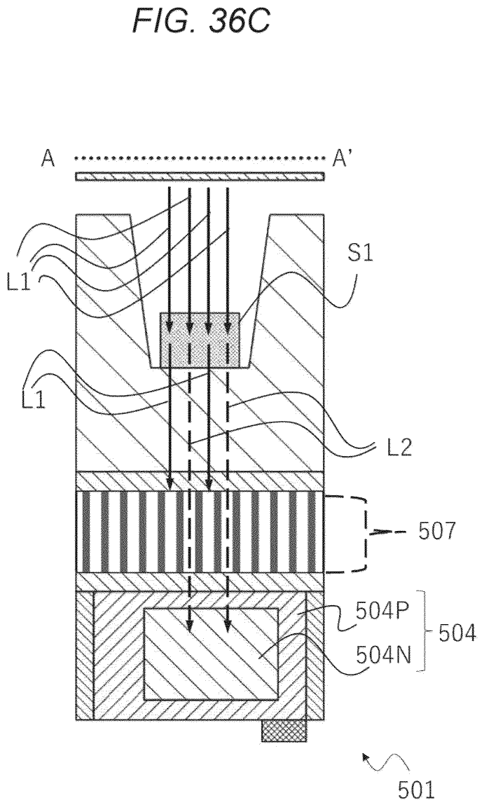

FIG. 36A illustrates the pixel unit 301 described in Example 1.2 above. The pixel unit includes a multilayer film reflection filter 307 as a component for blocking the excitation light. In order to achieve a high excitation light cut ratio, it is necessary to increase film thickness of the multilayer film reflection filter, but as a trade-off, thickness of a pixel structure increases, and as the miniaturization is promoted, optical crosstalk tends to occur.

In order to more reliably prevent transmission of the excitation light, a thickness T1 of the multilayer film reflection filter 307 of the pixel unit 301 is considered to be, for example, about several μm (particularly, about 3 μm to 4 μm). The back-illuminated imaging element in the present disclosure including a polarizer instead of the multilayer film reflection filter has, for example, a configuration illustrated in FIG. 36B, and a thickness T2 of the polarizer may be, for example, 1 μm or less (particularly, about 0.2 μm to 0.3 μm).

As described above, height of a pixel can be reduced by using a polarizer as the excitation light blocking unit in the present disclosure. This is very advantageous for pixel miniaturization.

In addition, since the polarizer has a high excitation light cut ratio, improvement of the S/N ratio can be achieved by using the polarizer instead of the multilayer film reflection filter. This contributes to improvement of the fluorescence detection accuracy.

The polarizer transmits polarized light of either S-polarized light or P-polarized light incident in an arrangement direction and quenches the other. In the present disclosure, therefore, polarized light quenched by the polarizer may be applied to the analyte as excitation light. Fluorescence is generated as a result of the radiation. The excitation light transmitted through the analyte holding unit (particularly, the nanowell) is quenched by the polarizer, and only the fluorescence is transmitted through the polarizer. The transmitted fluorescence is detected by the fluorescence detection photodiode. It is thus possible to selectively cut the excitation light through polarization, that is, it is possible to achieve a high excitation light cut ratio, by using the polarizer. In addition, since thickness of the polarizer is significantly smaller than the thickness of the multilayer film reflection filter, the height of the pixel can be reduced. Reducing the height of the pixel is also useful for suppressing optical crosstalk. In addition, the reduction in height of the pixel makes it possible to form a structure advantageous for the miniaturization of the pixel.

The back-illuminated imaging element in the present disclosure including a polarizer will be described hereinafter with reference to FIGS. 36B and 36C.

A pixel unit 501 illustrated in a right side of the drawing includes a well 503 and a fluorescence detection photodiode 504.

The pixel unit 501 includes an insulating film 502-1, a polarizer 507, and an insulating film 502-2 between the well 503 and the photodiode 504. The polarizer 507 corresponds to the excitation light blocking unit described above. The pixel unit 501 may thus be configured to have a multilayer structure in which the photodiode 504, the polarizer 507, and the well 503 are arranged in this order.

The imaging element according to the present disclosure may include a plurality of pixel units 501 arranged in a lattice pattern as described in Example 1-1 above. That is, as illustrated in a left plan view of the drawing, a back-illuminated imaging element 500 may include a plurality of pixel units 501 arranged in a lattice pattern.

In the drawing, the well 503, the photodiode 504 (an N region 504N and a P region 504P), a gate electrode unit 505, partitions 506, and a transparent substrate 508 may be the same as the well 303, the photodiode 304 (the N region 304N and the P region 304P), the gate electrode unit 305, the partitions 306, and the transparent substrate 308 described in Example 2-1 above, and the description also applies to this example.

The pixel unit 501 includes a polarizer 507 between the well 503 and the fluorescence detection photodiode 504. The polarizer 507 may be stacked on the well 503 via the insulating film 502-1. In addition, the polarizer 507 may be stacked on the fluorescence detection photodiode 504 via the insulating film 502-2. These insulating films may be, for example, SiO₂.

As illustrated in FIG. 36C, the analyte S1 is irradiated with the excitation light L1 (solid line arrows) that is the polarized light.

Part of the emitted excitation light passes through the analyte S1, travels through the well 503, and reaches the polarizer 507. The excitation light L1 that is polarized light is quenched by the polarizer 507. As a result, the excitation light L1 does not reach the fluorescence detection photodiode 504. By irradiating the analyte S1 with the excitation light L1, fluorescence L2 (broken line arrows) is generated. The fluorescence L2 passes through the polarizer 507 and reaches the fluorescence detection photodiode 504.

As described above, the polarizer can selectively cause the fluorescence to reach the photodiode. This contributes to improvement of the fluorescence detection accuracy.

### 1.3.2 Example 3-2 (Polarizer Pattern)

In the present disclosure, the polarizer is not used to obtain polarization information. One polarizer, therefore, may be stacked in one pixel unit, but one polarizer may be stacked over a plurality of pixel units. That is, one polarizer may be provided in such a way as to cover detection units of two or more pixel units. In addition, an orientation of the polarizer with respect to the photodiode may be any orientation.

For example, as illustrated in (a) of FIG. 37, one polarizer may be arranged for four photodiodes PD1 to PD4 in an oblique direction with respect to a pixel arrangement direction, or as illustrated in (b) of the drawing, one polarizer may be arranged for the four photodiodes PD1 to PD4 in a horizontal or vertical direction with respect to the pixel arrangement direction.

Furthermore, as illustrated in (c) and (d) of the drawing, one polarizer may be arranged for each photodiode in an oblique direction or in a horizontal or vertical direction with respect to the pixel arrangement direction.

### 1.3.3 Example 3-3 (Example of Case where Non-Polarized Light is Used)

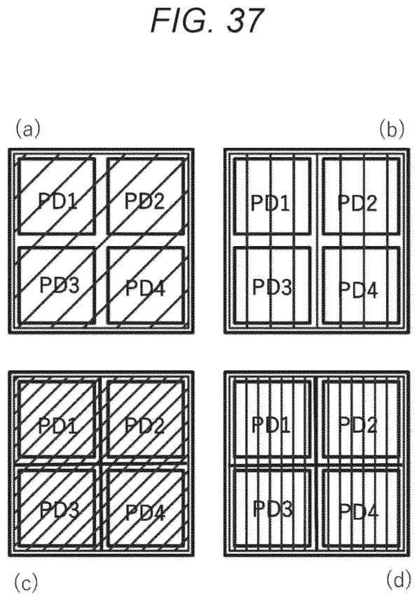

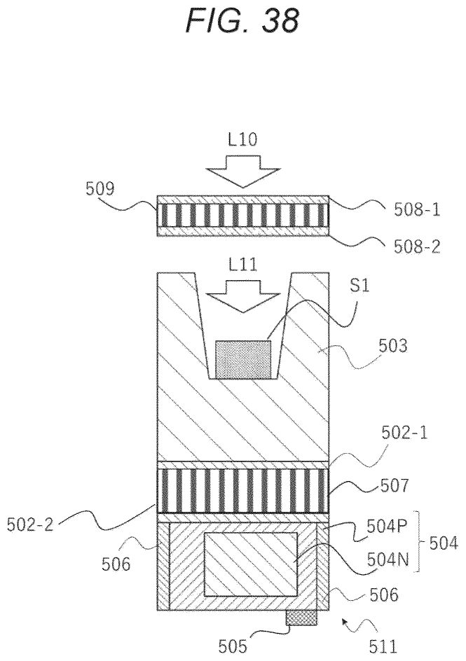

In the present disclosure, the light emitted from the light source may be non-polarized light. In this case, a polarizer that uses the non-polarized light as polarized light may be disposed on an optical path from the light source to an analysis unit. A configuration example in this case will be described with reference to FIG. 38.

In one embodiment, a polarizer may be stacked on the transparent substrate. For example, as illustrated in the drawing, a polarizer 509 may be provided between transparent substrates 508-1 and 508-2, that is, a multilayer structure of a transparent substrate, a polarizer, and a transparent substrate may be formed. Non-polarized light L10 is emitted from a normal light source and reaches the multilayer structure. Light L11, which is the non-polarized light L10 that has passed through the multilayer structure, is polarized light. The polarized light L11 is applied to the analyte.

Note that a polarizer may be stacked on one transparent substrate.

A pixel unit 511 illustrated in the drawing is the same as the pixel unit 501 described in Example 3-1 above. The polarizer 507 stacked on the photodiode 504 of the pixel unit 501 has optical characteristics of quenching the polarized light L11. As a result, the excitation light can be blocked as described in Example 3-1.

For example, a polarizer is mounted on a cover glass as the transparent substrate. The polarizer stacked on the photodiode is configured such that transmission/quenching characteristics become opposite to transmission/quenching characteristics of the polarizer on the cover glass for polarized light. That is, the polarized light transmitted by the polarizer on the cover glass is quenched by the polarizer stacked on the photodiode.

As described above, a light source that emits non-polarized light can be used by disposing a polarizer that transmits specific polarized light on an optical path between the light source and the well and disposing a polarizer that quenches the specific polarized light on an optical path between the well and the fluorescence detection photodiode.

### 1.3.4 Example 3-4 (Combination of Polarizer and Excitation Light Absorption Filter)

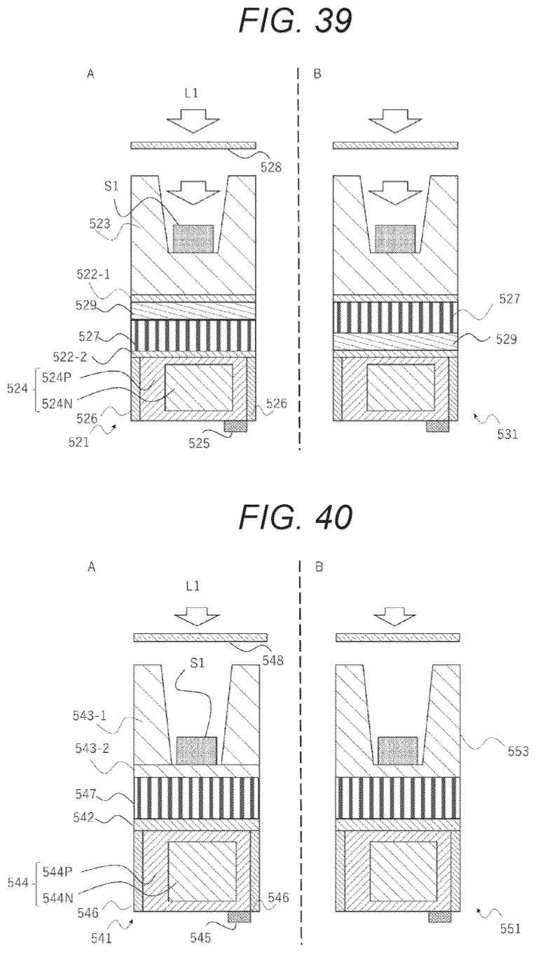

The pixel unit of the imaging element in the present disclosure may include an excitation light absorption filter in addition to the polarizer. These combinations can more reliably block the excitation light. A configuration example of a pixel unit of an imaging element having such a combination will be described with reference to FIG. 39.

A pixel unit 521 of an imaging element illustrated in A of the drawing includes a well 523 and a fluorescence detection photodiode 524.

The pixel unit 521 includes an insulating film 522-1, an absorption filter (for example, a multilayer film reflection filter) 529, a polarizer 527, and an insulating film 522-2 between the well 523 and the photodiode 524. The absorption filter 529 and the polarizer 527 correspond to the excitation light blocking unit described above. The pixel unit 521 may thus be configured to have a multilayer structure in which the photodiode 523, the polarizer 527, the absorption filter 529, and the well 523 are arranged in this order.

In the drawing, the well 523, the photodiode 524 (an N region 524N and a P region 524P), a gate electrode unit 525, partitions 526, and a transparent substrate 528 may be the same as the well 303, the photodiode 304 (the N region 304N and the P region 304P), the gate electrode unit 305, the partitions 306, and the transparent substrate 308 described in Example 2-1 above, and the description also applies to this example.

The pixel unit 521 further includes the absorption filter 529 in addition to the polarizer 527. The excitation light cut ratio, therefore, can be further increased.

In addition, a position of the polarizer 527 and a position of the absorption filter 529 only need to be between the well 523 and the fluorescence detection photodiode 524 (particularly, on an optical path of the excitation light between the well 523 and the fluorescence detection photodiode 524), and may be appropriately changed.

For example, as with an imaging element 531 illustrated in B of the drawing, the pixel unit 521 may have a multilayer structure in which the photodiode 523, the absorption filter 529, the polarizer 527, and the well 523 are arranged in this order.

### 1.3.5 Example 3-5 (Material of Analyte Holding Unit)

The pixel unit of the imaging element in the present disclosure includes an analyte holding portion as described above, particularly a well in which an analyte is held. The analyte holding unit may be stacked on the excitation light blocking unit, and is stacked via, for example, an insulating film. Materials of the analyte holding unit and the insulating film may be any materials that do not adversely affect light to be analyzed, and can be appropriately selected by those skilled in the art. Preferably, the materials are ones that do not interfere with the polarization and has weather resistance to a reagent.

For example, the well 503 illustrated in FIG. 36B may include a material such as, for example, SiO₂, SiN, or a resin material.

In addition, as in a pixel unit 541 illustrated in A of FIG. 40, a material of a bottom surface 543-2 of the well may be different from a material of side walls 543-1 of the well. For example, the material of the bottom surface 543-2 of the well may be SiN or glass.

In addition, as illustrated in the drawing, the bottom surface 543-2 may be stacked on a polarizer 547 (or an absorption filter). That is, the bottom surface 543-2 may be used as an interlayer film between the well 543 (particularly, the well side walls) and a modulator 547.

Note that a photodiode 544 (an N region 544N and a P region 544P), a gate electrode unit 545, partitions 546, and a transparent substrate 548 may be the same as the photodiode 304 (the N region 304N and the P region 304P), the gate electrode unit 305, the partitions 306, and the transparent substrate 308 described in Example 2-1 above, and the description also applies to this example.

In addition, as in a pixel unit 551 illustrated in B of the drawing, an entire well 553 may include SiN or glass.

Note that components of the pixel unit 551 other than the well illustrated in B of the drawing are the same as those in A of the drawing.

### 1.3.6 Example 3-6 (Plasmonic Filter)

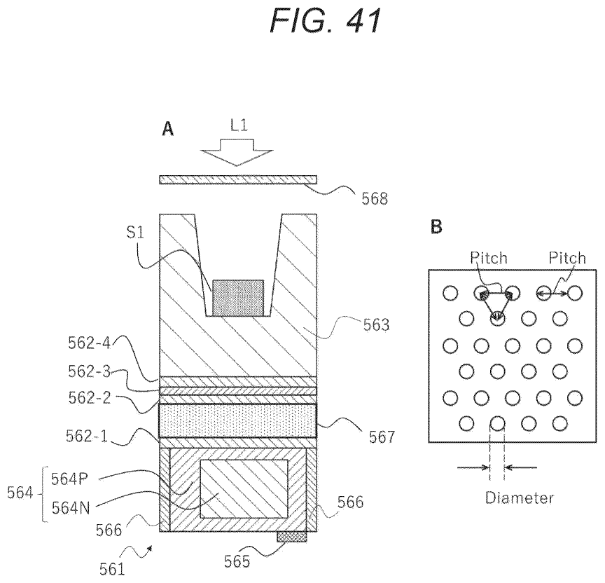

The pixel unit of the imaging element in the present disclosure may include a plasmonic filter as the excitation light blocking unit. In the present disclosure, wavelength selectivity of the plasmonic filter may be used to selectively block the excitation light and cause the fluorescence to reach the photodiode. A configuration example in which a plasmonic filter is used will be described with reference to FIG. 41.

A pixel unit 561 of an imaging element illustrated in A of the drawing includes a well 563 and a fluorescence detection photodiode 564.

The pixel unit 561 includes an insulating film 562-1, a plasmonic filter 567, and insulating films 562-2 to 562-4 between the well 563 and the photodiode 564. The plasmonic filter 567 corresponds to the excitation light blocking unit described above. The pixel unit 561 may thus be configured to have a multilayer structure in which the photodiode 564, the plasmonic filter 567, and the well 563 are arranged in this order.

In the drawing, the well 563, the photodiode 564 (an N region 564N and a P region 564P), a gate electrode unit 565, partitions 566, and a transparent substrate 568 may be the same as the well 303, the photodiode 304 (the N region 304N and the P region 304P), the gate electrode unit 305, the partitions 306, and the transparent substrate 308 described in Example 2-1 above, and the description also applies to this example.

The plasmonic filter 567 is a filter that selectively blocks excitation light by a surface plasmon resonance effect and that transmits fluorescence. The filter may include, for example, a metal, and may include, for example, Al or Cu, but is not limited thereto. The filter may have holes arranged in an array (also referred to as a hole array) as illustrated in a schematic cross-sectional view in B of the drawing.

A wavelength of the light to be blocked can be adjusted by adjusting a pitch and/or a diameter of the holes. The pitch and the diameter may have dimensions illustrated in B of the drawing. The pitch means intervals at which a unit structure of the holes appears. As a result of these adjustments, for example, transmission characteristics for light in a wavelength range of visible light to near-infrared light can be adjusted.

Materials of insulating films 562-1 to 562-4 may be appropriately selected by those skilled in the art.

Although three insulating films exist between the well and the plasmonic filter in the drawing, the number of insulating films need not be three. The number of insulating films provided between these may be one or more, and may be, for example, one, two, three, four, or five.

Although one insulating film exists between the photodiode and the plasmonic filter in the drawing, the number of insulating films need not be one. The number of insulating films provided between these may be one or more, and may be, for example, one, two, three, four, or five.

With respect to a material of each insulating film, as described above, each insulating film may be, for example, a silicon oxide film, a nitrogen-containing silicon oxide film, a silicon nitride film, an oxygen-containing silicon nitride film, or a metal oxide film. Each insulating film may be an insulating film subjected to high-density plasma processing.

### 1.3.7 Example 3-7 (Fabry-Perot Resonator Spectroscopy)

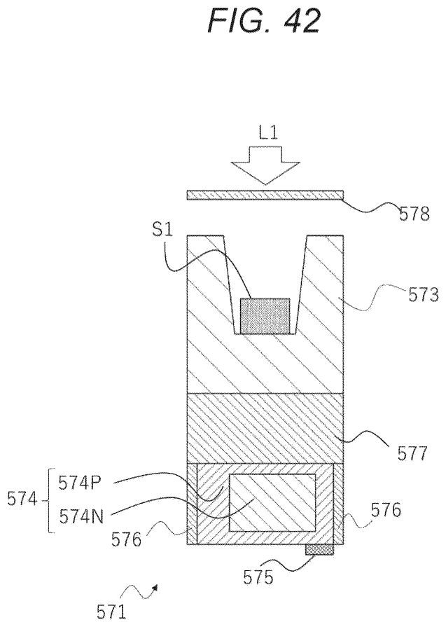

The pixel unit of the imaging element in the present disclosure may include a film having a Fabry-Perot structure (hereinafter also referred to as an FP structure) as the excitation light blocking unit. In the present disclosure, wavelength selectivity of a film having the FP structure may be used to selectively block the excitation light and cause the fluorescence to reach the photodiode. A configuration example in which a film having the FP structure is used will be described with reference to FIG. 42.

An imaging element 571 illustrated in the drawing includes a well 573 and a fluorescence detection photodiode 574.

The pixel unit 571 includes a film 577 having the FP structure between the well 573 and the photodiode 574. The film 577 having the FP structure corresponds to the excitation light blocking unit described above. The pixel unit 571 may thus be configured to have a multilayer structure in which the photodiode 574, the film 577 having the FP structure, and the well 573 are arranged in this order.

In the drawing, the well 573, the photodiode 574 (an N region 574N and a P region 574P), a gate electrode unit 575, partitions 576, and a transparent substrate 578 may be the same as the well 303, the photodiode 304 (the N region 304N and the P region 304P), the gate electrode unit 305, the partitions 306, and the transparent substrate 308 described in Example 2-1 above, and the description also applies to this example.

The film 577 having the FP structure blocks excitation light in a wavelength-selective manner by Fabry-Perot resonator spectroscopy. As the film having the FP structure, for example, a film having a high refractive material multilayer film structure, such as a TiO₂/SiO₂-based multilayer film or a PolySi/SiO₂-based multilayer film may be used. The film having the FP structure can selectively cut only the excitation light with a film thickness smaller than that of the multilayer film reflection filter.

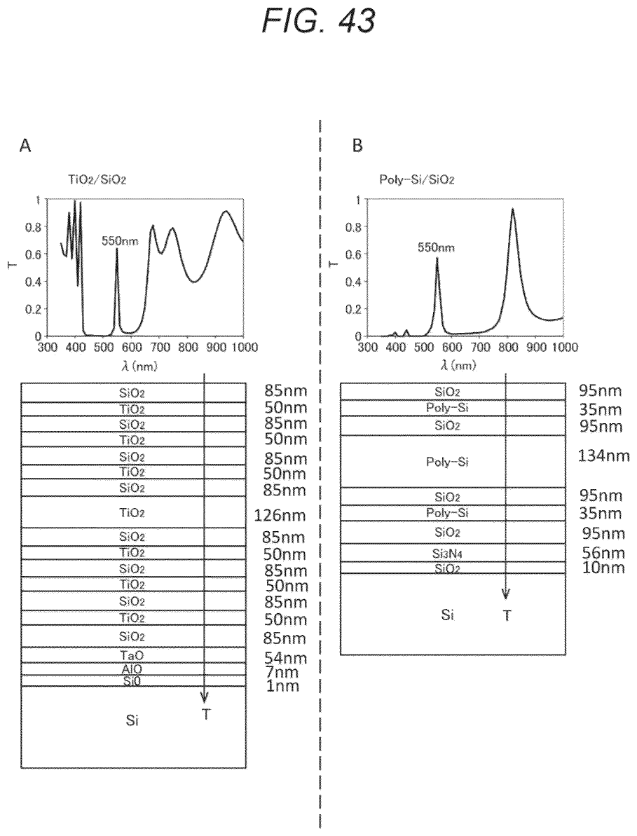

Optical characteristics of the film having the FP structure can be adjusted by adjusting thickness and a material of each layer constituting the film, the number of layers, and the like. This will be described hereinafter with reference to FIG. 43.

A of the drawing illustrates a graph (upper) showing transmittance of a TiO₂/SiO₂-based multilayer film at each wavelength and a schematic cross-sectional view (lower) of the multilayer film. In the graph, a horizontal axis λ (nₘ) represents wavelength λ of light incident on the multilayer film, and a vertical axis T represents a ratio (that is, transmittance T) of light transmitted through the multilayer film among the light incident on the multilayer film.

As illustrated in the drawing, the multilayer film is an 18-layer film, and the thickness of each layer is illustrated on the right of each layer. Total thickness of the multilayer film is 1168 nm.

Transmittance of light traveling in a direction indicated by arrows in the drawing (percentage of light reaching Si) through the film having the multilayer structure in the drawing is plotted on the graph.

The transmittance of the multilayer film has a peak in the vicinity of 550 nm. A half-value width at the peak is about 10 nm.

The transmittance of excitation light of 530 nm through the multilayer film is 1/60 or less with respect to the transmittance of fluorescence of 550 nm.

It is assumed that fluorescence with a wavelength of 553 nm is generated by irradiation of an analyte with excitation light with a wavelength of 532 nm (half-value width of about 30 nm). In this case, the multilayer film can selectively transmit fluorescence generated by irradiating the analyte with excitation light and selectively block the excitation light.

B of the drawing illustrates a graph (upper) showing transmittance of a PolySi/SiO₂-based multilayer film at each wavelength and a schematic cross-sectional view (lower) of the multilayer film. In the graph, a horizontal axis λ (nₘ) represents wavelength λ of light incident on the multilayer film, and a vertical axis T represents a ratio (that is, transmittance T) of light transmitted through the multilayer film among the light incident on the multilayer film.

As illustrated in the drawing, the multilayer film is a nine-layer film, and the thickness of each layer is illustrated on the right of each layer. Total thickness of the multilayer film is 650 nm.

Transmittance of light traveling in a direction indicated by arrows in the drawing (percentage of light reaching Si) through the film having the multilayer structure in the drawing is plotted on the graph.

The transmittance of the multilayer film has a peak in the vicinity of 550 nm. A half-value width at the peak is about 20 nm.

The transmittance of excitation light of 530 nm through the multilayer film is 1/10 or less with respect to the transmittance of fluorescence of 550 nm.

It is assumed that fluorescence with a wavelength of 553 nm is generated by irradiation of an analyte with excitation light with a wavelength of 532 nm (half-value width of about 30 nm). In this case, the multilayer film can selectively transmit fluorescence generated by irradiating the analyte with excitation light and selectively block the excitation light.

As described above, the film having the FP structure can selectively transmit fluorescence and selectively block excitation light. In addition, by adjusting the configuration of the film, light having a desired wavelength can be selectively transmitted or blocked.

### 1.4 Configuration Example Relating to PD Vertically Stacked Structure

### 1.4.1 Example 4-1 (Basic Configuration Example)

As described in 1. above, the back-illuminated imaging element according to the present disclosure includes a fluorescence detection unit that detects fluorescence generated by irradiation of an analyte with excitation light. In one implementation, the fluorescence detection unit may include two or more photodiodes. The two or more photodiodes may be arranged in such a way as to form a vertically stacked structure between the analyte holding unit and the wiring layer.

A configuration example of the back-illuminated imaging element in the present disclosure including the fluorescence detection unit including two or more photodiodes will be described hereinafter with reference to the drawings.

In order to analyze a biological sample, it might be desirable to use two or more fluorescent substances. For example, with respect to DNA sequencing, throughput is higher in a case where two-color method chemistry using two types of fluorescent substances or four-color method chemistry using four types of fluorescent substances is employed than in a case where one-color method chemistry using one type of fluorescent substance is employed. This is because in the case of the one-color method chemistry, it is necessary to wash away the fluorescent substance for specifying each base and a blocking agent in order to use the fluorescent substance and it is necessary to perform staining and image reading more times, but in the two-color method or the four-color method, these times can be reduced.

Since the fluorescence detection unit of the back-illuminated imaging element according to the present disclosure includes two or more photodiodes, light of two or more different wavelengths can be simultaneously detected. This improves throughput in biological sample analysis in which two or more fluorescent substances are used. For example, a base sequence of a nucleic acid (for example, DNA or RNA) can be determined with a smaller number of times of staining, and throughput of base sequencing can be improved.

The two or more photodiodes are preferably arranged in such a way as to form a vertically stacked structure between the analyte holding unit and the wiring layer. This is particularly suitable, for example, for nucleic acid sequencing. In nucleic acid sequencing, since extension of DNA fixed at a specific position in a plane is detected by fluorescence, it is easy to obtain fluorescence information indicating the extension of the DNA in a case where photodiodes are vertically stacked at the same position in the light receiving surface (in a case where photodiodes are stacked in a depth direction). In addition, size of the pixel unit on the light receiving surface can be reduced by the vertically stacked structure.

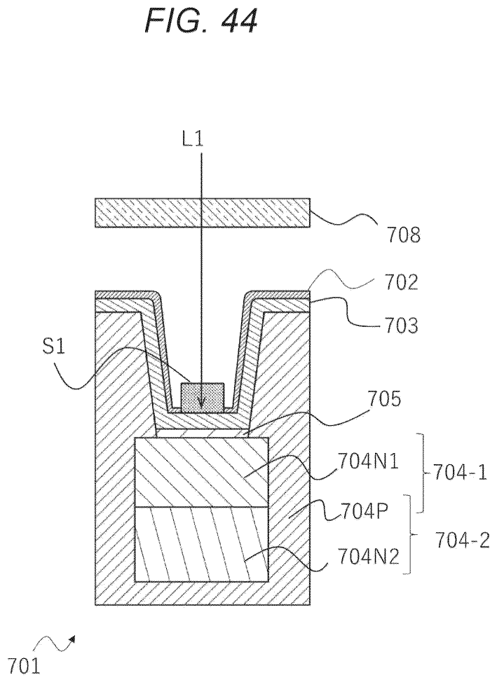

The back-illuminated imaging element will be described hereinafter with reference to FIG. 44. The drawing is a schematic cross-sectional view of an example of the back-illuminated imaging element.

A pixel unit 701 includes a well 703. As illustrated in the drawing, the well 703 may be covered with an insulating film 702.

A shape of the well 703 is rectangular in the drawing, but may be another polygonal shape, or may be circular, elliptical, or the like, instead.

The well 703 (and the insulating film 702 covering the well) may be configured to hold an analyte, that is, corresponds to the analyte holding unit.

The description of the well 103 and the insulating film 102 described in 1.1 above also applies to the well 703 and the insulating film 702.

The pixel unit 701 includes two photodiodes 704-1 and 704-2. The photodiodes may be, for example, Si photodiodes.

The photodiode 704-1 includes an N region 704N1 and a P region 704P.

The photodiode 704-2 includes an N region 704N2 and the P region 704P.

In the drawing, the N region 704N1 of the photodiode 704-1 and the N region 704N2 of the photodiode 704-2 are in contact with each other, but these regions may be separated from each other. In the latter case, these two N regions may be separated by the P region.

As illustrated in the drawing, the N regions 704N1 and 704N2 may be surrounded by the P region 704P.

In the present disclosure, since the pixel unit includes two photodiodes in this manner, two light beams having different wavelengths can be detected in one pixel unit.

In the present disclosure, the number of photodiodes included in one pixel unit is not limited to two, and may be three or more, instead, as described later.

As illustrated in the drawing, the two photodiodes 704-1 and 704-2 are arranged in a direction perpendicular to the light receiving surface, that is, vertically stacked on each other. The vertically stacked structure is particularly suitable for DNA sequencing, for example, as described above.

The pixel unit 701 may include an insulating film 707. The well 703 and the photodiodes 704 may be stacked via the insulating film 707.

Although not illustrated in the drawing, the pixel unit may be separated from other unit pixels by partitions. The description of the partitions 106 described in 1.1 above also applies to the partitions in the present example.

Although not shown in the drawing, the pixel unit includes gate electrode units connected to the photodiodes 704-1 and 704-2, respectively. The gate electrode units may be polysilicon as described in 1.1 above. A configuration example of the gate electrode units will be described later in 4.2.

Pixel units 701 may be arranged in an array to form an imaging element. The imaging element is of a back-illuminated type, that is, a wiring layer (not illustrated) is provided on a side of the photodiodes opposite a fluorescence incident side.

The well 703 is provided on one side of the photodiodes 704, and the wiring layer is provided on an opposite side of the photodiodes 704. That is, the pixel unit 701 has a multilayer structure in which the wiring layer, the detection units (photodiodes), and the analyte holding unit (well) are arranged in this order. With such a configuration, the imaging element in the present disclosure can obtain a larger fluorescence signal, which contributes to improvement of the fluorescence detection accuracy.

### 1.4.2 Example 4-2 (Configuration Example of Imaging Element Including Two Photodiodes)

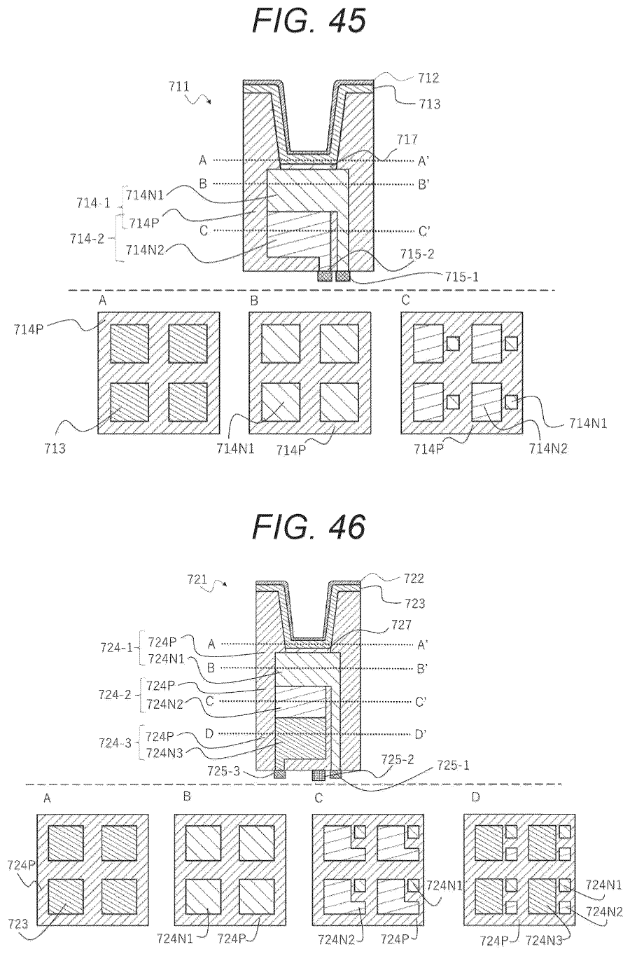

The imaging element described in Example 4-1 above includes gate electrode units connected to the two photodiodes, respectively. In a case where the two photodiodes form a vertically stacked structure, light receiving area of the photodiode farther from the well (lower photodiode) is smaller than light receiving area of the photodiode closer to the well (upper photodiode). As a result, it is easy to form the gate electrodes respectively allocated to these two photodiodes, and in particular, the area of the pixel unit (the area of a surface parallel to the light receiving surface) can be reduced. This will be described hereinafter with reference to FIG. 45.

A schematic cross-sectional view of a pixel unit of an imaging element including two photodiodes is illustrated in an upper part of the drawing. The cross-sectional view is a schematic cross-sectional view of a plane perpendicular to the light receiving surface.

A pixel unit 711 includes a well 713 and insulating films 712 and 717. These are the same as the well 703 and the insulating films 702 and 707 described in Example 4-1 above, and the description also applies to the present example.

The pixel unit includes two photodiodes 714-1 and 714-2. These photodiodes may be, for example, Si photodiodes.

The photodiode 714-1 includes an N region 714N1 and a P region 714P.

The photodiode 714-2 includes an N region 714N2 and the P region 714P.

In the drawing, the N region 714N1 and the N region 714N2 are in contact with each other, but these regions may be separated from each other, instead. In the latter case, these two N regions may be separated by the P region.

As illustrated in the drawing, the N regions 714N1 and 714N2 may be surrounded by the P region 714P.

As illustrated in the drawing, the two photodiodes 714-1 (particularly the N region 714N1) and 714-2 (particularly the N region 714N2) are arranged in a direction perpendicular to the light receiving surface, that is, vertically stacked on each other. The vertically stacked structure is particularly suitable for DNA sequencing, for example, as described above.

The photodiode 714-1 is connected to a gate electrode unit 715-1. The photodiode 714-2 is connected to a gate electrode unit 715-2. These gate electrode units may include polysilicon as described in 1.1 above. These gate electrode units are connected to the wiring layer. In addition, the imaging element is of a back-illuminated type, and the wiring layer is disposed below in the drawing. That is, the imaging element has a multilayer structure in which the well, the photodiodes, and the wiring layer are arranged in this order.

Schematic views of cross sections taken along line A-A′, line B-B′, and line C-C′ in the upper part of the drawing are illustrated in diagram A, diagram B, and diagram C in a lower part of the drawing, respectively. These schematic cross-sectional views are schematic cross-sectional views in a case where the plurality of pixel units 711 is arranged in an array to form an imaging element.

As illustrated in diagram A, the well 713 appears in the cross-sectional view taken along line A-A′.

As illustrated in diagram B, the P region 714P and the N region 714N1 constituting the photodiode 714-1 appear in the cross-sectional view taken along line B-B′.

As illustrated in diagram C, the N region 714N1 appears in the cross-sectional view taken along line C-C′ in addition to the P region 714P and the N region 714N2 constituting the photodiode 714-2. In addition, as can be seen from diagrams B and C, area of the N region 714N2 of the photodiode 714-2 is smaller than area of the N region 714N1 of the photodiode 714-1. The gate electrode unit 715-1 connected to the photodiode 714-1 is disposed using the difference in the area, so that size of the pixel unit is not increased.

As described above, since the two photodiodes are provided in such a way as to form a vertically stacked structure, two fluorescence beams having different wavelengths can be detected in one pixel unit.

In addition, in the vertically stacked structure, by making the light receiving area of the photodiode positioned below smaller than that of the photodiode positioned above, the gate electrode units can be provided in such a way as not to increase the pixel unit in size. This contributes to downsizing of the imaging element.

### 1.4.3 Example 4-3 (Configuration Example of Imaging Element Including Three Photodiodes)

The imaging elements described in Examples 4-1 and 4-2 above include two photodiodes, which constitute the fluorescence detection unit. The fluorescence detection unit in the pixel unit of the imaging element in the present disclosure may include three or more photodiodes. A pixel unit of an imaging element including a fluorescence detection unit including three photodiodes will be described hereinafter with reference to FIG. 46.

A schematic cross-sectional view of a pixel unit of an imaging element including three photodiodes is illustrated in an upper part of the drawing. The cross-sectional view is a schematic cross-sectional view of a plane perpendicular to the light receiving surface. Note that although three gate electrode units are illustrated in the cross-sectional view, these are illustrated for convenience in order to better understand the present example, and are not completely consistent with cross-sectional views illustrated in a lower part of the drawing.

A pixel unit 721 includes a well 723 and insulating films 722 and 727. These are the same as the well 703 and the insulating films 702 and 707 described in Example 4-1 above, and the description also applies to the present example.

The pixel unit includes two photodiodes 724-1, 724-2, and 724-3. These photodiodes may be, for example, Si photodiodes.

The photodiode 724-1 includes an N region 724N1 and a P region 724P.

The photodiode 724-2 includes an N region 724N2 and the P region 724P.

The photodiode 724-3 includes an N region 724N3 and the P region 724P.

In the drawing, the N region 724N1 and the N region 724N2 are in contact with each other, or the N region 724N2 and the N region 724N3 are in contact with each other. In the present disclosure, these N regions may be separated from each other, instead. In a case where the two N regions are separated from each other, these two N regions may be separated by the P region.

As illustrated in the drawing, the N regions 724N1, 724N2, and 724N3 may be surrounded by the P region 724P.

As illustrated in the drawing, the three photodiodes 724-1 (particularly the N region 724N1), 724-2 (particularly the N region 714N2), and 724-3 (particularly the N region 714N3) are arranged in a direction perpendicular to the light receiving surface, that is, vertically stacked on each other. The vertically stacked structure is particularly suitable for DNA sequencing, for example, as described above.

The photodiode 724-1 is connected to a gate electrode unit 725-1. The photodiode 724-2 is connected to a gate electrode unit 725-2. The photodiode 724-3 is connected to a gate electrode unit 725-3. These gate electrode units may include polysilicon as described in 1.1 above. These gate electrode units are connected to the wiring layer. In addition, the imaging element is of a back-illuminated type, and the wiring layer is disposed below in the drawing. That is, the imaging element has a multilayer structure in which the well, the photodiodes, and the wiring layer are arranged in this order.

Schematic views of cross sections taken along line A-A′, line B-B′, line C-C′, and line D-D′ in the upper part of the drawing are illustrated in diagram A, diagram B, diagram C, and diagram D in the lower part of the drawing, respectively. These schematic cross-sectional views are schematic cross-sectional views in a case where a plurality of the pixel units 721 is arranged in an array to form an imaging element.

As illustrated in diagram A, the well 723 appears in the cross-sectional view taken along line A-A′.

As illustrated in diagram B, the P region 724P and the N region 724N1 constituting the photodiode 724-1 appear in the cross-sectional view taken along line B-B′.

As illustrated in diagram C, the N region 724N1 appears in the cross-sectional view taken along line C-C′ in addition to the P region 724P and the N region 724N2 constituting the photodiode 724-2. As illustrated in diagram D, the N region 724N1 and the N region 724N2 appear in the cross-sectional view taken along line D-D′ in addition to the P region 724P and the N region 724N3 constituting the photodiode 724-3.

In addition, as can be seen from diagrams B, C, and D, area of the N region 724N2 of the photodiode 724-2 is smaller than area of the N region 724N1 of the photodiode 724-1. In addition, area of the N region 724N3 of the photodiode 724-3 is smaller than area of the N region 724N2 of the photodiode 724-2. The gate electrode units 725-1 and 725-2 are disposed using the difference in the area, so that size of the pixel unit is not increased.

As described above, since the three photodiodes are provided in such a way as to form a vertically stacked structure, three fluorescence beams having different wavelengths can be detected in one pixel unit.

In addition, in the vertically stacked structure, by making the light receiving area of the photodiode positioned below smaller than that of the photodiode positioned above, the gate electrode units can be provided in such a way as not to increase the pixel unit in size. This contributes to downsizing of the imaging element.

### 1.4.4 Example 4-4 (Sensitivity Verification in Case where Two Photodiodes are Provided)

Sensitivity of each photodiode was verified for the pixel unit 711 described in Example 4-2 above. The sensitivity verification was performed on the basis of an integral value of light intensity of light in a fluorescence wavelength region of a generally used fluorescent substance (red light (668 nm) and green light (545 nm)) up to a Si depth of 3 μm.

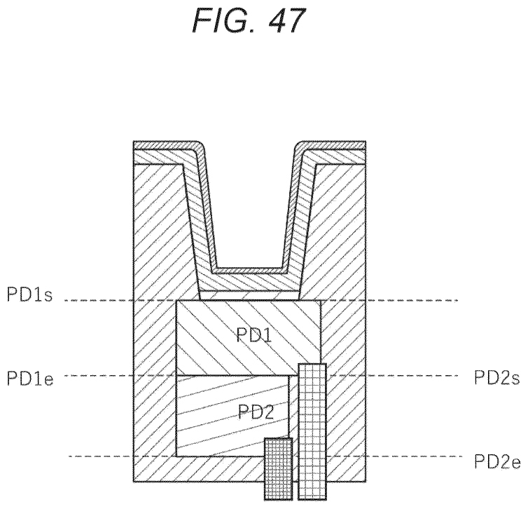

As illustrated in FIG. 47, an upper end of an upper photodiode PD1 (N region) is defined as PD1s, and a lower end is defined as PDle. Similarly, an upper end of a lower photodiode PD2 (N region) is defined as PD2s, and a lower end is defined as PD2e.

In FIG. 48, a relationship between the light intensity of the red light and the light intensity of the green light and the Si depth is plotted. The plot also shows absorption of these light beams. As shown in the plot, the light intensity of the red light and the green light decreases as the depth increases. Accordingly, the absorption of these light beams increases.

From the plot, the integrated values of the light intensity of PD1 at positions from PD1s to PDle are obtained. Similarly, from the plot, the integrated values of the light intensity of PD2 at positions from PD2s to PD2e are obtained. In this verification, it was assumed that PD1 was used to detect the green light and PD2 was used to detect the red light.

A G/R rate in a case where the position of PD1 was fixed and PD2 was moved to various positions was calculated. In addition, a G/R rate in a case where the position of PD2 was fixed and PD1 was moved to various positions was calculated.

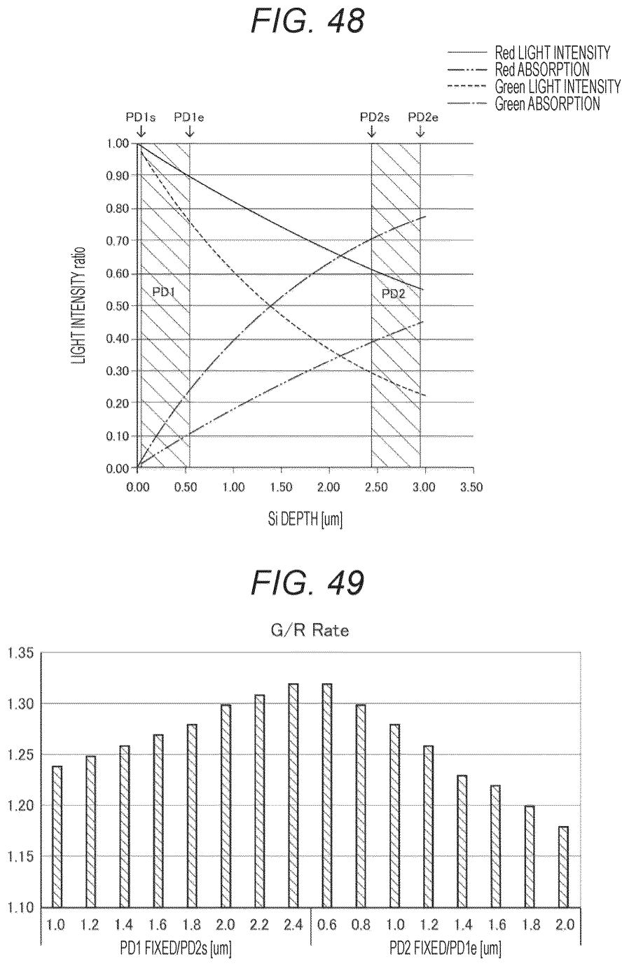

FIG. 49 illustrates the calculated G/R rate. As can been seen from these results, in a case where the position of PD1 is fixed, the deeper the position of PD2s is, the higher the G/R ratio is. In addition, in a case where the position of PD2 is fixed, the shallower the position of PDle is, the higher the G/R ratio is. For this reason, it is considered desirable to separate the positions of the two PDs in the depth direction (a direction perpendicular to the light receiving surface) in Si in order to increase the sensitivity.

In addition, it can be seen that a sensitivity ratio of 1.32 is achieved in this case for the Si depth of 3 μm.

In addition, on the basis of these results, in the present disclosure, a photodiode closer to the wiring layer among the two or more photodiodes may be configured to detect fluorescence of a longer wavelength.

### 1.5. Manufacturing Method

### 1.5.1 Example 1 of Manufacturing Method (Imaging Element Including PD Having Well Structure)

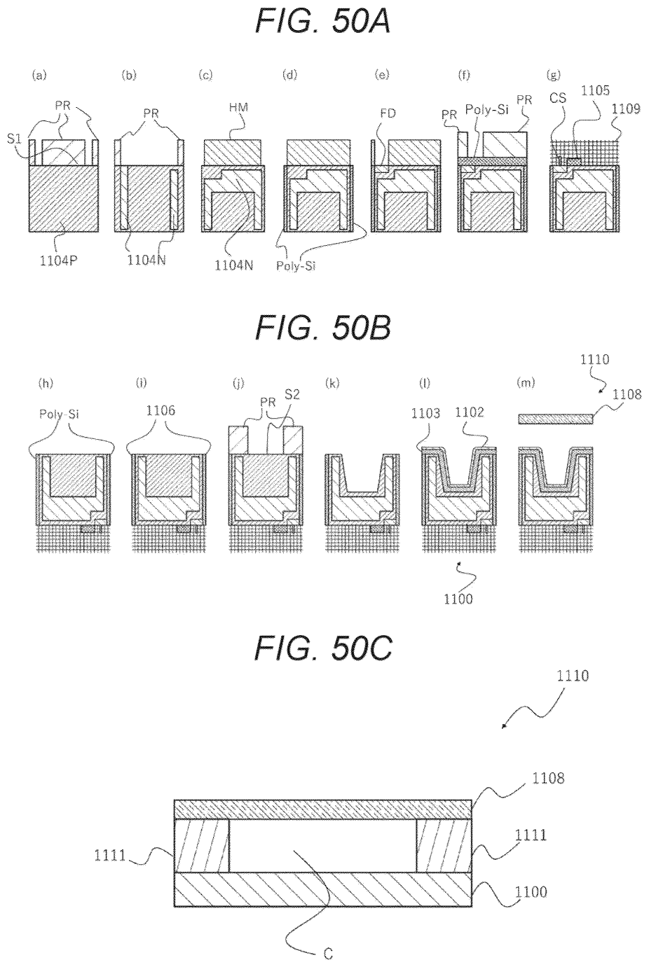

The imaging element according to the present disclosure can be manufactured, for example, by applying a technique known in the technical field related to imaging elements. An example of a method for manufacturing the imaging element according to the present disclosure will be described hereinafter with reference to FIGS. 50A and 50B. In this example, an example of a manufacturing flow of an imaging element 1100 having the same structure as the imaging element 100 described in Example 1-1 above will be described.

FIGS. 50A and 50B are schematic diagrams for explaining a flow diagram of a method for manufacturing the back-illuminated imaging element according to the present disclosure.

As illustrated in (a) of FIG. 50A, a Si wafer 1104P for forming a photodiode is prepared, and a photoresist PR is applied to a surface S1 of the wafer in such a way as to draw a predetermined pattern. For example, as illustrated in the drawing, the photoresist PR may be applied to regions other than regions in which the side walls of the well are to be formed.

After the application, the N region 1104N of the photodiode is embedded by photolithography as illustrated in (b) and (c) of the drawing. A shape of the N region 1104N to be formed may be appropriately designed by those skilled in the art in accordance with, for example, a structure of the well to be formed or a position, a shape, or the like of FD or a gate electrode unit 1105, which will be described later.

Next, as illustrated in (c) of the drawing, a hard mask HM is formed on the surface S1.

After the hard mask HM is formed, Poly-Si is embedded in the regions where the partitions are to be formed, as illustrated in (d) of the drawing. The embedding may be performed by, for example, dry etching.

Next, after the removal of the hard mask HM, FD is formed in a part of the P region 1104P as illustrated in (e) of the drawing. Thereafter, as illustrated in (f) of the drawing, a Poly-Si layer is formed on the surface S1, and then the photoresist PR is stacked on the Poly-Si layer. The photoresist PR may be applied in such a way as to draw a predetermined pattern. For example, as illustrated in the drawing, the photoresist PR can be stacked in regions other than the gate electrode unit.

After the stacking, only the Poly-Si to be the gate electrode unit 1105 is left by photolithography and dry etching, and the other part of the Poly-Si layer is removed as illustrated in (g) of the drawing. Thereafter, a wiring layer 1109 is provided on the surface S1, and a contact CS connected to FD is also formed.

Next, as illustrated in (h) of FIG. 50B, the wafer is turned over.

Next, as illustrated in (i) of the drawing, tungsten is embedded in the Poly-Si in portions of the surface S2 of the wafer in which the partitions are to be formed to form the partitions 1106. The embedding may be performed by, for example, wet etching.

After the partitions 1106 are formed, as illustrated in (j) of the drawing, the photoresist PR is stacked on the surface S2 in such a way as to draw a predetermined pattern. The photoresist may be stacked, for example, in portions other than a portion in which the well is to be formed.

Next, as illustrated in (k) of the drawing, dry etching is performed to form a well structure in the P region.

After the formation of the well structure, as illustrated in (l) of the drawing, a color filter (or multilayer film reflection filter) 1103 for forming the well surface is formed, and an insulating film 1102 is further formed on the color filter 1103. An imaging element 1100 is thus manufactured.

As illustrated in (m) of the drawing, the imaging element 1100 is combined with various members forming the flow channel unit, such as a transparent substrate 1108, to form a flow channel unit 1110 for analyzing a biological sample.

FIG. 50C illustrates a schematic cross-sectional view of an example of the flow channel unit 1110 for analyzing a biological sample. As illustrated in the drawing, the flow channel unit 1110 for analyzing a biological sample includes the back-illuminated imaging element 1100 and the transparent substrate 1108 according to the present disclosure, and the imaging element 1100 and the transparent substrate 1108 are arranged to form a flow channel C. In order to form a space of the flow channel C, the back-illuminated imaging element 1100 and the transparent substrate 1108 may be connected via walls 1111.

The flow channel C is a flow channel through which a biological sample containing an analyte flows. Since the biological sample flows through the flow channel C, the analyte is captured in the well (particularly, the bottom surface of the well) of the imaging element 1100. The analyte is then irradiated with excitation light, and fluorescence generated by the radiation of the excitation light is detected by the imaging element 1100.

### 1.5.2 Example 2 of Manufacturing Method (Imaging Element Including Multilayer Film Reflection Filter)

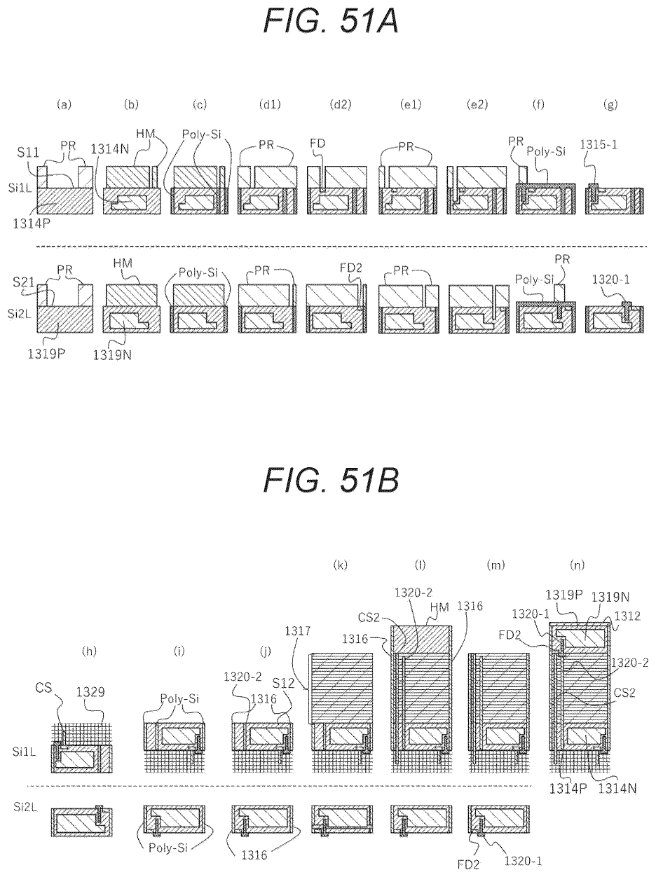

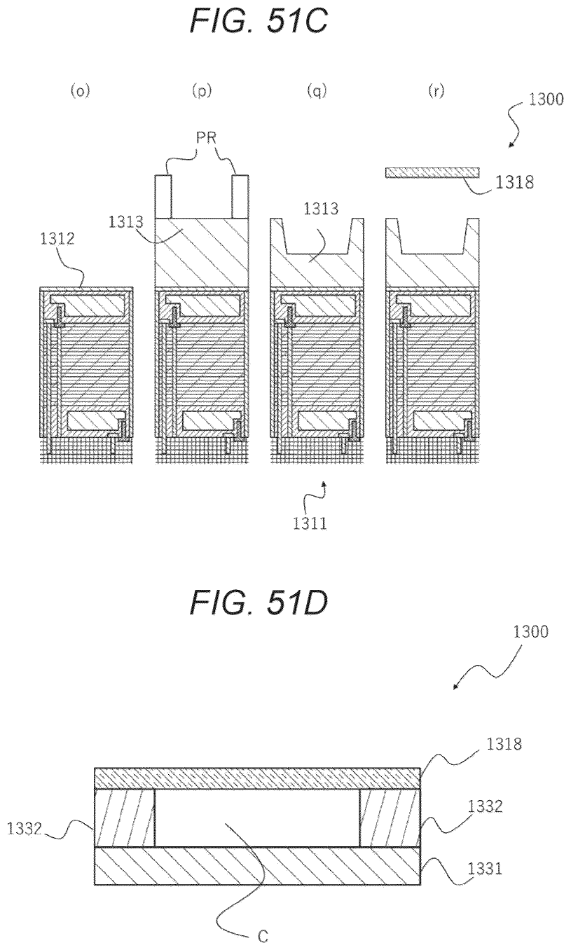

An example of a method for manufacturing the imaging element according to the present disclosure will be described hereinafter with reference to FIGS. 51A to 51C. In this example, an example of a manufacturing flow of an imaging element including the plurality of pixel element 311 described in Example 2-2 above will be described.

Each pixel unit 311 includes a fluorescence detection photodiode 314 and an excitation light detection photodiode 319. In order to form the pixel unit 311, therefore, these two photodiodes are manufactured using different Si wafers. After the structure of each photodiode is formed, the two photodiodes are then stacked. In the following FIGS. 51A and 51B, a flow diagram for forming the fluorescence detection photodiode is illustrated in a row of Si1L, and a flow diagram for forming the excitation light detection photodiode is illustrated in a row of Si2L. In FIG. 51C, a flow diagram after the two photodiodes are bonded together is illustrated. Details of these flows will be described below.

As illustrated in (a) of FIG. 51A, a Si wafer 1314P for forming the fluorescence detection photodiode and a Si wafer 1319P for forming the excitation light detection photodiode are prepared.

The photoresist PR is applied to a surface S11 of the Si wafer 1314P in such a way as to draw a predetermined pattern. For example, as illustrated in the drawing, the photoresist PR may be applied to regions other than a region in which the N region is to be formed.

The photoresist PR is also applied to a surface S21 of the Si wafer 1319P in such a way as to draw a predetermined pattern. For example, as illustrated in the drawing, the photoresist PR may be applied to regions other than a region in which the N region is to be formed.

An N region 1314N is embedded by photolithography in the Si wafer 1314P for forming the fluorescence detection photodiode as illustrated in (b) of the drawing.

Similarly, an N region 1319N is also embedded in the Si wafer 1319P by photolithography.

After the N regions are formed, a hard mask layer HM is formed in such a way as to draw a predetermined pattern. The hard mask layer HM is formed in such a way as to cover regions other than a region in which the Poly-Si is to be embedded.

As illustrated in (c) of the drawing, the Poly-Si is embedded in both wafers. The embedding may be performed by, for example, dry etching.

After the embedding, the hard mask is removed, and then, as illustrated in (dl) of the drawing, a photoresist PR for forming FD is formed on the surfaces S11 and S21 of the wafers in such a way as to draw predetermined patterns. As illustrated in the drawing, the photoresist PR is stacked in regions other than a region in which FD (Si1L row) or FD2 (Si2L row) is to be formed. Next, ion implantation processing is performed. As illustrated in (d2) of the drawing, FD (Si1L row) is formed by the ion implantation processing in the region of the Si wafer 1314P in which the photoresist is not stacked. Similarly, FD2 (Si2L row) is formed on the Si wafer 1319P by the ion implantation processing. Thereafter, the photoresist PR is removed.

The photoresist PR is stacked in regions other than regions where the gate electrode units are to be embedded. Next, as illustrated in (d2) of the drawing, dry etching processing is performed to scrape the regions where the gate electrode units are to be embedded. Thereafter, the photoresist PR is removed.

Next, as illustrated in (e1) of the drawing, a photoresist PR for forming a gate electrode unit is formed on the surfaces S11 and S21 of the wafers in such a way as to draw predetermined patterns. As illustrated in the drawing, the photoresist PR is stacked in regions other than the regions where the gate electrode units are to be embedded. Next, as illustrated in (e2) of the drawing, dry etching processing is performed to scrape the regions where the gate electrode units are to be embedded. Thereafter, the photoresist PR is removed. Thereafter, the photoresist PR is removed, and the entire exposed surfaces of Si are oxidized in the removal. As a result of the oxidation, thermal oxide films are formed in the regions where the gate electrode units are to be embedded. Inner surfaces of trenches are thus covered with the thermal oxide films.

Next, as illustrated in (f) of the drawing, Poly-Si layers are formed on the surface S11 and the surface S21. Accordingly, Poly-Si is embedded also in the trenches.

Thereafter, a photoresist is formed on the Poly-Si layer of each wafer. The photoresist is formed in the portions where the gate electrode units are to be formed.

After the photoresist is formed, the Poly-Si layers in the regions where the photoresist is not formed are removed by photolithography and dry etching processing, and gate electrode units 1315-1 and 1320-1 are formed as illustrated in (g) of the drawing.

Thereafter, as illustrated in (h) of FIG. 51B, a contact CS and a wiring layer 1329 are formed on the surface S11 of the wafer of the fluorescence detection photodiode. As a result, the wiring layer 1329 and the gate electrode unit 1315-1 are connected to each other.

In addition, an adhesive material to be used for bonding, which will be described later, is applied to the surface S21 of the wafer of the excitation light detection photodiode.

After the formation of the wiring layer, the wafer of the fluorescence detection photodiode is turned over as illustrated in (i) of the drawing.

Also for the excitation light detection photodiode, the wafer may be turned over, and the bonding described later may be performed in this state.

Next, as illustrated in (j) of the drawing, the Poly-Si embedded in the wafer is dry-etched to embed tungsten in both the photodiode for fluorescence detection and the photodiode for excitation light detection. As a result, partitions 1316 are formed, and a gate electrode unit 1320-2 is also formed in the P region of the fluorescence detection photodiode.

Thereafter, as illustrated in (k) of the drawing, a multilayer film reflection filter 1317 is formed on the surface S12 (a surface opposite a surface on which the wiring layer is to be formed) of the fluorescence detection photodiode. In order to form the multilayer film reflection filter, two insulating films having different refractive indexes may be alternately formed.

After the formation of the multilayer film reflection filter, a hard mask HM is formed on the multilayer film reflection filter as illustrated in (l) of the drawing. The hard mask HM is formed in regions other than the regions where the partitions and the gate electrode unit are to be formed. Then, after the hard mask is formed, dry etching processing is performed, and then tungsten is embedded. As a result, the partitions 1316 and the gate electrode unit 1320-2 are formed in the multilayer film reflection filter. In addition, a contact CS2 to be connected to FD2 at a later stage is also formed.

Next, as illustrated in (m) of the drawing, the hard mask HM is removed.

After the removal of the hard mask, as illustrated in (n) of the drawing, an excitation light detection photodiode is stacked on the multilayer film reflection filter via the adhesive amount. The gate electrode unit 1320-1 and the gate electrode unit 1320-2 are connected to each other by the stacking. In addition, FD2 is connected to CS2.

Next, as illustrated in (o) of FIG. 51C, an insulating film 1312 is formed on the excitation light detection photodiode.

Thereafter, as illustrated in (p) of the drawing, a material 1313 for forming a well is stacked on the insulating film 1312, and the photoresist is stacked on the material in such a way as to draw a predetermined pattern. The photoresist is stacked in regions other than the region where the well is to be formed.

Thereafter, as illustrated in (q) of the drawing, the material 1313 is etched by, for example, dry etching processing to form the well. An imaging element in which the pixel units 1311 are arranged is thus manufactured.

As illustrated in (r) of the drawing, the imaging element is combined with various members forming a flow channel unit, such as a transparent substrate 1318, to form a flow channel unit 1300 for analyzing a biological sample.

FIG. 51D illustrates a schematic cross-sectional view of an example of the flow channel unit 1300 for analyzing a biological sample. As illustrated in the drawing, the flow channel unit 1300 for analyzing a biological sample includes a back-illuminated imaging element 1331 and the transparent substrate 1318 according to the present disclosure, and the imaging element 13313 and the transparent substrate 1318 are arranged to form a flow channel C. In order to form a space of the flow channel C, the back-illuminated imaging element 1331 and the transparent substrate 1318 may be connected via walls 1332.

The flow channel C is a flow channel through which a biological sample containing an analyte flows. Since the biological sample flows through the flow channel C, the analyte is captured in the well (particularly, the bottom surface of the well) of the imaging element 1331. The analyte is then irradiated with excitation light, and fluorescence generated by the radiation of the excitation light is detected by the imaging element 1331.

### 1.5.3 Example 3 of Manufacturing Method (Imaging Element Including Multilayer Film Reflection Filter)

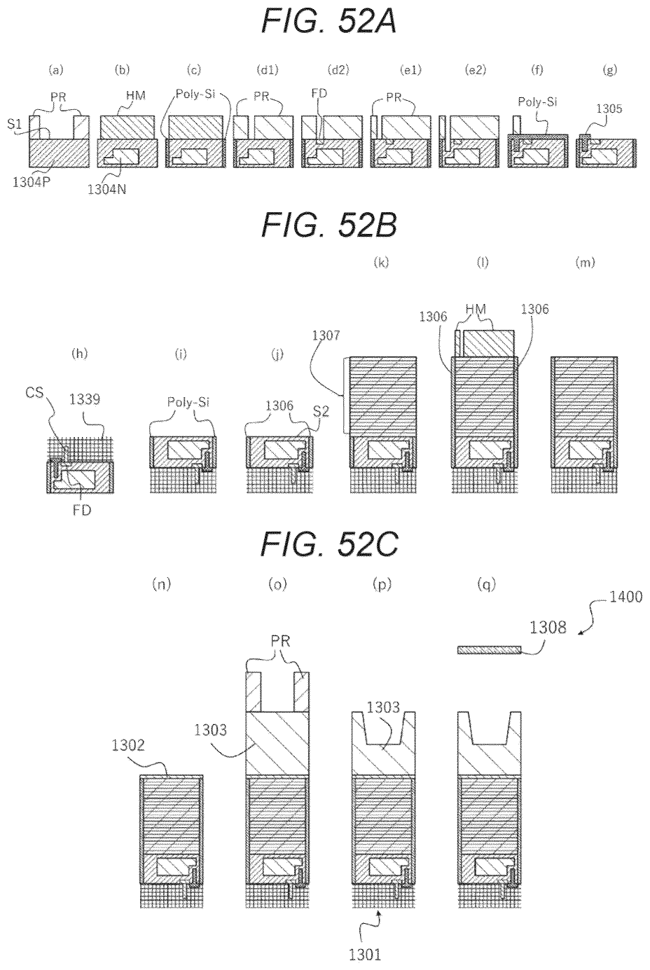

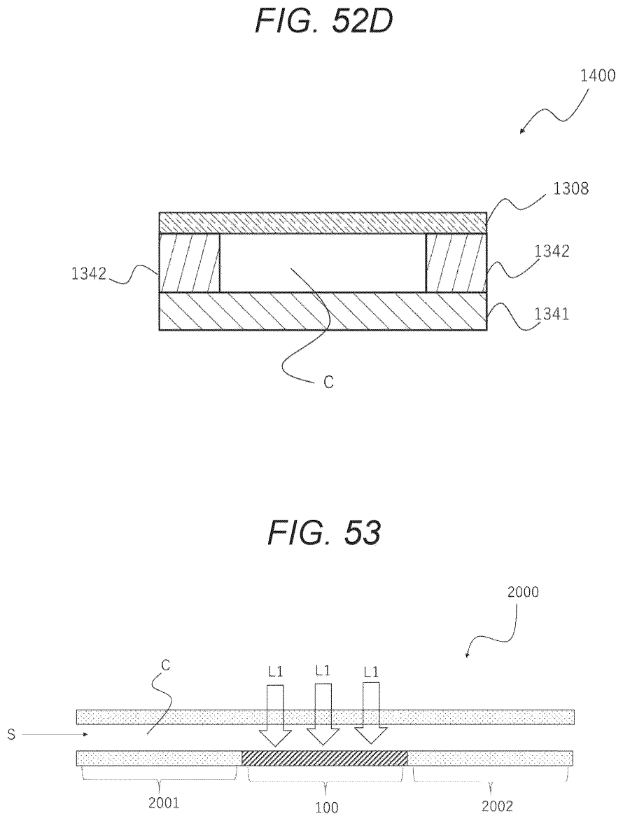

An example of a method for manufacturing the imaging element according to the present disclosure will be described hereinafter with reference to FIGS. 52A to 52C. In this example, an example of a manufacturing flow of an imaging element 1341 including the plurality of pixel element 301 described in Example 2-1 above will be described. Details of these flows will be described hereinafter.

As illustrated in (a) of FIG. 52A, a Si wafer 1304P for forming the fluorescence detection photodiode is prepared.

The photoresist PR is applied to a surface S1 of the Si wafer 1304P in such a way as to draw a predetermined pattern. For example, as illustrated in the drawing, the photoresist PR may be applied to regions other than a region in which the N region is to be formed.

An N region 1314N is embedded by photolithography in the Si wafer 1314P for forming the fluorescence detection photodiode as illustrated in (b) of the drawing.

After the N regions are formed, a hard mask layer HM is formed in such a way as to draw a predetermined pattern. The hard mask layer HM is formed in such a way as to cover regions other than a region in which the Poly-Si is to be embedded.

As illustrated in (c) of the drawing, the Poly-Si is embedded in both wafers. The embedding may be performed by, for example, dry etching.

After the embedding, the hard mask is removed, and then, as illustrated in (d1) of the drawing, a photoresist PR for forming FD is formed on the surfaces S11 of the wafer in such a way as to draw a predetermined pattern. As illustrated in the drawing, the photoresist PR is stacked in regions other than a region in which FD is to be formed. Next, ion implantation processing is performed. As illustrated in (d2) of the drawing, FD is formed by the ion implantation processing in the region of the Si wafer 1304P in which the photoresist is not stacked. Thereafter, the photoresist PR is removed.

Next, as illustrated in (e1) of the drawing, a photoresist PR for forming a gate electrode unit is formed on the surface S1 of the wafer in such a way as to draw a predetermined pattern. As illustrated in the drawing, the photoresist PR is stacked in regions other than the regions where the gate electrode units are to be embedded. Next, as illustrated in (e2) of the drawing, dry etching processing is performed to scrape the regions where the gate electrode units are to be embedded. Thereafter, the photoresist PR is removed, and the entire exposed surfaces of Si are oxidized in the removal. As a result of the oxidation, thermal oxide films are formed in the regions where the gate electrode units are to be embedded. Inner surfaces of trenches are thus covered with the thermal oxide films.

Next, as illustrated in (f) of the drawing, a Poly-Si layer is formed on the surface S1. Accordingly, Poly-Si is embedded also in the trenches.

Thereafter, a photoresist is formed on the Poly-Si layer. The photoresist is formed in the portion where the gate electrode unit is to be formed.

After the photoresist is formed, the Poly-Si layers in the regions where the photoresist is not formed are removed by photolithography and dry etching processing, and a gate electrode unit 1305 is formed as illustrated in (g) of the drawing.

Thereafter, as illustrated in (h) of FIG. 51B, a wiring layer 1339 is formed on the surface S1 of the wafer of the fluorescence detection photodiode. As a result, the wiring layer 1339 is formed, and the contact CS connected to FD is also formed.

After the formation of the wiring layer, the wafer of the fluorescence detection photodiode is turned over as illustrated in (i) of the drawing.

Next, as illustrated in (j) of the drawing, the Poly-Si embedded in the wafer is dry-etched to embed tungsten in both the photodiode for fluorescence detection and the photodiode for excitation light detection.

Thereafter, as illustrated in (k) of the drawing, a multilayer film reflection filter 1307 is formed on the surface S2 (a surface opposite a surface on which the wiring layer is to be formed) of the fluorescence detection photodiode. In order to form the multilayer film reflection filter, two insulating films having different refractive indexes may be alternately formed.

After the formation of the multilayer film reflection filter, a hard mask HM is formed on the multilayer film reflection filter as illustrated in (l) of the drawing. The hard mask HM is formed in regions other than the regions where the partitions are to be formed. Then, after the hard mask is formed, dry etching processing is performed, and then tungsten is embedded. As a result, the partitions 1306 are formed in the multilayer film reflection filter.

Next, as illustrated in (m) of the drawing, the hard mask HM is removed.

After the removal of the hard mask, an insulating film 1302 is formed on the multilayer film reflection filter as illustrated in (n) of FIG. 52C.

Thereafter, as illustrated in (o) of the drawing, a material 1303 for forming a well is stacked on the insulating film 1302, and the photoresist is further stacked on the material in such a way as to draw a predetermined pattern. The photoresist is stacked in regions other than the region where the well is to be formed.

Thereafter, as illustrated in (p) of the drawing, the material 1303 is etched by, for example, dry etching processing to form the well. An imaging element in which the pixel units 1301 are arranged is thus manufactured.

As illustrated in (q) of the drawing, the imaging element is combined with various members forming a flow channel unit, such as a transparent substrate 1308, to form a flow channel unit 1400 for analyzing a biological sample.

FIG. 52D illustrates a schematic cross-sectional view of an example of the flow channel unit 1400 for analyzing a biological sample. As illustrated in the drawing, the flow channel unit 1200 for analyzing a biological sample includes a back-illuminated imaging element 1341 and the transparent substrate 1308 according to the present disclosure, and the imaging element 1341 and the transparent substrate 1308 are arranged to form a flow channel C. In order to form a space of the flow channel C, the back-illuminated imaging element 1341 and the transparent substrate 1308 may be connected via walls 1342.

The flow channel C is a flow channel through which a biological sample containing an analyte flows. Since the biological sample flows through the flow channel C, the analyte is captured in the well (particularly, the bottom surface of the well) of the imaging element 1341. The analyte is then irradiated with excitation light, and fluorescence generated by the radiation of the excitation light is detected by the imaging element 1341.

### 2. Second Embodiment (Flow Channel Unit for Analyzing Biological Sample)

The present disclosure provides a flow channel unit including a back-illuminated imaging element according to the present disclosure. The flow channel unit may be used, for example, to analyze a biological sample, but may be used for other purposes.

The biological sample may be a sample containing an analyte. The analyte may generate fluorescence as a result of the excitation light radiation described above. The analyte is, for example, a nucleic acid, and more specifically, may be DNA or RNA. That is, the flow channel unit in the present disclosure may be a flow channel unit for analyzing a nucleic acid.

The nucleic acid analysis may be a sequence analysis of a nucleic acid. That is, the flow channel unit in the present disclosure may be a flow channel unit for analyzing a nucleic acid. The flow channel unit is particularly suitable for determining a base sequence of a nucleic acid.

The analyte may be a biological substance other than a nucleic acid, and may be, for example, a protein, a lipid, a peptide, or a sugar. The analyte may be an antibody or an antigen.

The analyte may be a bioparticle, that is, for example, a cell or a non-cellular bioparticle. The cell may be, for example, a blood cell, but may be another cell. In addition, the non-cellular bioparticle may be an extracellular vesicle, particularly an exosome, a microvesicle, or the like.

The analyte may be a bacterium or a virus.

A configuration example of the flow channel unit according to the present disclosure will be described with reference to FIG. 53. A flow channel unit 2000 illustrated in the drawing includes a back-illuminated imaging element 100 according to the present disclosure, a sample supply flow channel unit 2001 that supplies a sample (particularly, a liquid sample) to the imaging element 100, and a sample discharge flow channel unit 2002 that discharges the sample from the imaging element. These three elements are fluidly connected to each other to form a flow channel C.

For example, the sample supply flow channel unit 2001 may be fluidly connected to a container storing a sample to be analyzed.

The sample discharge flow channel unit 2002 may be fluidly connected to, for example, a container that collects a waste liquid.

A sample flows in the flow channel C, and an analyte (for example, nucleic acid) contained in the sample is captured in the well of the imaging element 100.

In the well, a chemical or biological reaction that uses the analyte may occur. For example, a nucleic acid extension reaction may occur in the well. The nucleic acid extension reaction may be a reaction for nucleotide sequencing. The sequencing may be Sanger sequencing or next generation sequencing. The next generation sequencing may be pyrosequencing, sequencing by synthesis, or sequencing by ligation.

The flow channel C is designed in such a way as to flow a biological sample S. The flow channel C may be formed in a flow channel structure such as a microchip (a chip including a flow channel on the order of micrometers) or a flow cell. Width of the flow channel C may be, for example, 1 mm or less, and may be particularly 10 μm or more and 1 mm or less, for example, 20 μm or more and 500 μm or less. The flow channel C and the flow channel structure including the flow channel C may include a material such as plastic or glass.

In order to irradiate the analyte in the well of the imaging element with the excitation light, at least a part of the flow channel may be transparent, and in particular, a flow channel portion through which L1 in the drawing passes may be transparent. As described above, in the flow channel unit and the imaging element according to the present disclosure, the portion through which the excitation light and the fluorescence pass may be transparent.

### 3. Third Embodiment (Biological Sample Analysis System)

The present disclosure also provides a biological sample analysis system including the back-illuminated imaging element according to the present disclosure or the flow channel unit according to the present disclosure. In the system, the back-illuminated imaging element or the flow channel unit according to the present disclosure may be interchangeably incorporated in the system. That is, the back-illuminated imaging element or the flow channel unit according to the present disclosure may be used as a disposable element in the system.

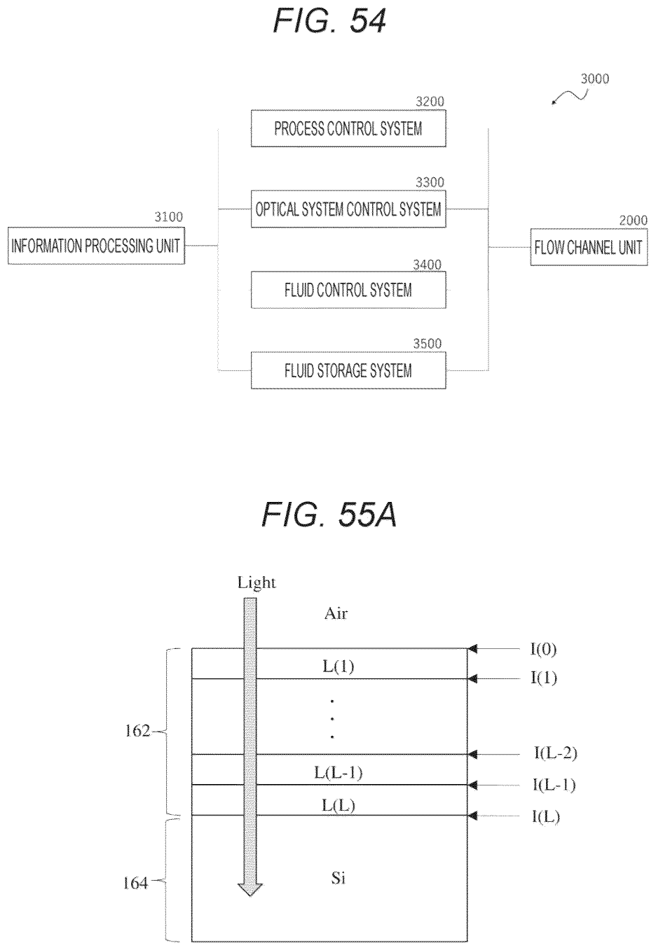

A configuration example of the system will be described with reference to FIG. 54. A biological sample analysis system 3000 illustrated in the drawing includes an information processing unit 3100, a process control system 3200, an optical system control system 3300, a fluid control system 3300, and a fluid storage system 3400 in addition to a flow channel unit 2000 according to the present disclosure (or a back-illuminated imaging element according to the present disclosure).

The flow channel unit 2000 according to the present disclosure and the imaging element included in the flow channel unit are as described in 1, and 2. above.

The information processing unit 3100 includes, for example, a processing section that processes various types of data (for example, fluorescence data), and a storage section storing various types of data. The processing section can execute analysis processing on the basis of fluorescence data obtained by the imaging element of the flow channel unit. The analysis processing may be, for example, processing for determining a base sequence, but may be another type of processing.

The information processing unit 3100 may be configured to be capable of outputting various types of data (for example, light data and images). For example, the information processing unit 3100 can output various types of data (for example, base sequence data, identification data regarding an analyte, or the like) generated on the basis of the fluorescence data. In addition, the information processing unit 3100 may be configured to be capable of accepting inputs of various types of data, and, for example, accepts an analysis instruction data from a user. The information processing unit 3100 may include an output section (for example, a display or the like) or an input section (for example, a keyboard or the like) for performing the output or the input.

The information processing unit 3100 may be configured as a general-purpose computer, and may be configured as an information processing device including, for example, a CPU (or GPU), a RAM, and a ROM. The information processing unit 3100 may be included in a housing provided with one or more of the process control system 3200, the optical system control system 3300, the fluid control system 3400, and the fluid storage system 3500, or may be outside the housing. In addition, various processes or functions to be executed by the information processing unit 3100 may be achieved by a server computer or a cloud connected via a network.

The process control system 3200 may be a system that controls processing (particularly biological or chemical processing) performed in the flow channel unit. The system performs, for example, temperature control in the flow channel unit and/or control of supply or discharge of a sample or a reagent into or from the flow channel unit. To perform the control, the process control system 3200 may include a temperature regulation system. In addition, in order to perform the control, the process control system 3200 may control another element 3300, 3400, or 3500.

The optical system control system 3300 may be configured to control irradiation of an analyte with excitation light and obtaining of fluorescence data generated by the radiation of the excitation light. The optical system control system 3300 may include, for example, a light radiation unit. The light radiation unit may include a light source section that emits light and a light guide optical system that guides the light to an irradiation position (for example, an analyte in a well provided in the imaging element). The light source section includes one or more light sources. A type of the light source(s), for example, is a laser light source or an LED. The light source section emits excitation light. In addition, the light source section may emit unpolarized light or may emit polarized light. A wavelength of the light emitted from each light source may be appropriately selected in such a way as to generate desired fluorescence. The light may have, for example, a wavelength of any of ultraviolet light, visible light, and infrared light. The light guide optical system includes, for example, optical components such as beam splitters, mirrors, or optical fibers. In addition, the light guide optical system may also include lenses for condensing light, including, for example, an objective lens.

The fluid control system 3400 controls supply of fluid to the flow channel unit 2000 and/or discharge of the fluid from the flow channel unit 2000. The fluid control system 3400 may include, for example, one or more pump units. The fluid control system 3400 may include a pump unit that controls the supply of the fluid to the flow channel unit 2000 and/or a pump unit that controls the discharge of the fluid from the flow channel unit 2000.

The fluid storage system 3500 may include a container containing a sample, a container containing a reagent, and a container containing the waste liquid, and may be configured to control these containers. For example, a temperature control device that detects and/or controls temperature in these containers may be included.

### (Detection Unit)

A detection unit 6102 includes at least one photodetector that detects light generated by radiating light onto a bioparticle. The light to be detected may be, for example, fluorescence or scattered light (for example, one or more of forward scattered light, backward scattered light, and side scattered light). Each photodetector includes one or more light receiving elements, and includes, for example, a light receiving element array. Each photodetector may include one or more photomultiplier tubes (PMTs) and/or photodiodes such as APDs and MPPCs as the light receiving elements. The photodetectors each include, for example, a PMT array in which a plurality of PMTs is arranged in a one-dimensional direction. In addition, the detection unit 6102 may include an imaging element such as a CCD or a CMOS. With the imaging element, the detection unit 6102 can obtain an image (for example, a bright-field image, a dark-field image, or a fluorescent image) of a bioparticle.

The detection unit 6102 includes a detection optical system that causes light of a predetermined detection wavelength to reach the corresponding photodetector. The detection optical system includes a spectroscopic unit such as a prism or a diffraction grating, or a wavelength separation unit such as a dichroic mirror or an optical filter. The detection optical system is configured to disperse light generated by irradiating the bioparticle with light, for example, and detect the dispersed light with a larger number of photodetectors than the number of fluorescent dyes with which the bioparticle is labeled. A flow cytometer including such a detection optical system is called a spectral flow cytometer. In addition, the detection optical system is configured to separate light corresponding to a fluorescence wavelength range of a specific fluorescent dye from the light generated by irradiating the bioparticle with light, for example, and cause the corresponding photodetector to detect the separated light.

In addition, the detection unit 6102 can include a signal processing section that converts an electrical signal obtained by a photodetector into a digital signal. The signal processing section may include an A/D converter as a device that performs the conversion. The digital signal obtained as a result of the conversion performed by the signal processing section can be transmitted to the information processing unit 6103. The digital signal can be handled by the information processing unit 6103 as data relating to light (hereinafter also referred to as “light data”). The light data may be, for example, light data including fluorescence data. More specifically, the light data may be light intensity data, and the light intensity may be light intensity data regarding light including fluorescence (may include features such as area, height, and width).

Note that the present disclosure can also have the following configurations.

[1]

A back-illuminated imaging element including
a plurality of pixel units, each of which includes at least:
an analyte holding unit configured to hold an analyte; and
a fluorescence detection unit that detects fluorescence generated by irradiating the analyte with excitation light.
   [2]

The back-illuminated imaging element according to [1], in which
the analyte holding unit has a shape of a well, and
the detection unit is provided in such a way as to cover a side surface of the well in addition to a bottom of the well.
   [3]

The back-illuminated imaging element according to [1] or [2], in which a trench is provided between the pixel units in the back-illuminated imaging element.

[4]

The back-illuminated imaging element according to any one of [1] to [3], in which two or more wells are connected to each other in such a way as to form a column structure.

[5]

The back-illuminated imaging element according to any one of [1] to [4], in which each pixel unit is provided with an electrode pair to which a voltage is applied in such a way as to adjust a position of the analyte.

[6]

The back-illuminated imaging element according to any one of [1] to [5], in which each pixel unit is provided with an excitation light blocking unit that prevents the excitation light from reaching the detection unit.

[7]

The back-illuminated imaging element according to [6], in which
the excitation light blocking unit includes a multilayer film reflection filter.
   [8]

The back-illuminated imaging element according to [7], in which the multilayer film reflection filter is disposed between the analyte holding unit and the detection unit.

[9]

The back-illuminated imaging element according to any one of [1] to [8], in which each pixel unit further includes an excitation light detection unit that detects the excitation light.

[10]

The back-illuminated imaging element according to [9], in which the back-illuminated imaging element is configured to process a signal obtained by the fluorescence detection unit using a signal obtained by the excitation light detection unit.

[11]

The back-illuminated imaging element according to any one of [6] to [10], in which
the excitation light blocking unit includes a polarizer, a plasmon filter, a metamaterial, or a multilayer film having a Fabry-Perot structure.
   [12]

The back-illuminated imaging element according to any one of [6] to [11], in which the excitation light blocking unit is configured to transmit the fluorescence.

[13]

The back-illuminated imaging element according to any one of [6] to [12], in which
the excitation light blocking unit includes a polarizer, and
the excitation light is polarized light.
   [14]

The back-illuminated imaging element according to any one of [6] to [12], in which
the excitation light blocking unit includes a polarizer, and
one polarizer is provided in such a way as to cover detection units of two or more pixel units.
   [15]

The back-illuminated imaging element according to any one of [1] to [14], in which the fluorescence detection unit includes two or more photodiodes.

[16]

The back-illuminated imaging element according to [15], in which the two or more photodiodes are arranged in such a way as to form a vertically stacked structure between the analyte holding unit and a wiring layer.

[17]

The back-illuminated imaging element according to or [16], in which a photodiode closer to the wiring layer among the two or more photodiodes is configured to detect fluorescence of a longer wavelength.

[18]

The back-illuminated imaging element according to any one of to [17], in which the two or more photodiodes form a two-layer structure or a three-layer structure.

[19]

A flow channel unit for analyzing a biological sample, the flow channel unit including:
a back-illuminated imaging element including a plurality of pixel units, each of which includes at least an analyte holding unit configured to hold an analyte and a fluorescence detection unit that detects fluorescence generated by irradiating the analyte with excitation light; and
a flow channel that supplies the biological sample to the analyte holding unit.
   [20]

A biological sample analysis system that analyzes a biological sample using a flow channel unit for analyzing a biological sample, the flow channel unit including:
a back-illuminated imaging element including a plurality of pixel units, each of which includes at least an analyte holding unit configured to hold an analyte and a fluorescence detection unit that detects fluorescence generated by irradiating the analyte with excitation light; and
a flow channel that supplies the biological sample to the analyte holding unit.

### REFERENCE SIGNS LIST

100 Imaging element
101 Pixel unit
102 Insulating film
103 Well
104 Photodiode
105 Gate electrode unit
106 Partitions
108 Transparent substrate

What is claimed is:

1. A back-illuminated imaging element, comprising:
a plurality of pixel units, each of which includes at least:
an analyte holding unit configured to hold an analyte; and
a fluorescence detection unit that detects fluorescence generated by irradiating the analyte with excitation light.

2. The back-illuminated imaging element according to claim 1, wherein
the analyte holding unit has a shape of a well, and
the detection unit is provided in such a way as to cover a side surface of the well in addition to a bottom of the well.

3. The back-illuminated imaging element according to claim 2, wherein a trench is provided between the pixel units in the back-illuminated imaging element.

4. The back-illuminated imaging element according to claim 2, wherein two or more wells are connected to each other in such a way as to form a column structure.

5. The back-illuminated imaging element according to claim 2, wherein each pixel unit is provided with an electrode pair to which a voltage is applied in such a way as to adjust a position of the analyte.

6. The back-illuminated imaging element according to claim 1, wherein each pixel unit is provided with an excitation light blocking unit that prevents the excitation light from reaching the detection unit.

7. The back-illuminated imaging element according to claim 6, wherein
the excitation light blocking unit includes a multilayer film reflection filter.

8. The back-illuminated imaging element according to claim 7, wherein the multilayer film reflection filter is disposed between the analyte holding unit and the detection unit.

9. The back-illuminated imaging element according to claim 1, wherein each pixel unit further includes an excitation light detection unit that detects the excitation light.

10. The back-illuminated imaging element according to claim 9, wherein the back-illuminated imaging element is configured to process a signal obtained by the fluorescence detection unit using a signal obtained by the excitation light detection unit.

11. The back-illuminated imaging element according to claim 6, wherein
the excitation light blocking unit includes a polarizer, a plasmon filter, a metamaterial, or a multilayer film having a Fabry-Perot structure.

12. The back-illuminated imaging element according to claim 11, wherein the excitation light blocking unit is configured to transmit the fluorescence.

13. The back-illuminated imaging element according to claim 6, wherein
the excitation light blocking unit includes a polarizer, and
the excitation light is polarized light.

14. The back-illuminated imaging element according to claim 6, wherein
the excitation light blocking unit includes a polarizer, and
one polarizer is provided in such a way as to cover detection units of two or more pixel units.

15. The back-illuminated imaging element according to claim 1, wherein the fluorescence detection unit includes two or more photodiodes.

16. The back-illuminated imaging element according to claim 15, wherein the two or more photodiodes are arranged in such a way as to form a vertically stacked structure between the analyte holding unit and a wiring layer.

17. The back-illuminated imaging element according to claim 16, wherein a photodiode closer to the wiring layer among the two or more photodiodes is configured to detect fluorescence of a longer wavelength.

18. The back-illuminated imaging element according to claim 15, wherein the two or more photodiodes form a two-layer structure or a three-layer structure.

19. A flow channel unit for analyzing a biological sample, the flow channel unit comprising:
a back-illuminated imaging element including a plurality of pixel units, each of which includes at least an analyte holding unit configured to hold an analyte and a fluorescence detection unit that detects fluorescence generated by irradiating the analyte with excitation light and a flow channel that supplies the biological sample to the analyte holding unit.

20. A biological sample analysis system that analyzes a biological sample using a flow channel unit for analyzing a biological sample, the flow channel unit comprising:
a back-illuminated imaging element including a plurality of pixel units, each of which includes at least an analyte holding unit configured to hold an analyte and a fluorescence detection unit that detects fluorescence generated by irradiating the analyte with excitation light; and
a flow channel that supplies the biological sample to the analyte holding unit.
